SEQUENCE LISTING (3 sequences)

SEQ ID NO: 1 - DNA, 19 bp, Artificial Sequence
  siRNA sequence
ugguuuacauguuguguga

SEQ ID NO: 2 - DNA, 21 bp, Artificial Sequence
  siRNA sequence
aaucuugccagcuuuccccuu

SEQ ID NO: 3 - RNA, 19 nt, Artificial Sequence
  siRNA sequence
ugguuuacauguuguguga

(19) United States
(12) Patent Application Publication
(10) Pub. No.: US 2010/0055169 A1
(43) Pub. Date: Mar. 4, 2010

(54) CATIONIC LIPIDS AND USES THEREOF

(75) Inventors: Prasad A. Dande, Evanston, IL (US); Todd M. Hansen, Grayslake, IL (US); Robert D. Hubbard, Lindenhurst, IL (US); Aparna V. Sarthy, Waukegan, IL (US); Yu Shen, Gurnee, IL (US); Lu Tian, Blue Bell, PA (US); Carol K. Wada, Evanston, IL (US); Xiaobin Zhao, Waukegan, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/557,230

(22) Filed: Sep. 10, 2009

Related U.S. Application Data
(63) Continuation-in-part of application No. 12/425,266, filed on Apr. 16, 2009.
(60) Provisional application No. 61/045,349, filed on Apr. 16, 2008.

(51) Int. Cl. A61K 9/127; A61K 31/7088; A61K 38/00; C11C 3/00

(52) U.S. Cl. 424/450; 514/44 R; 514/44 A; 514/2; 554/61

(57) ABSTRACT

Cationic lipids, cationic lipid based drug delivery systems, ways to make them and methods of treating diseases using them are disclosed.

This application is a continuation-in-part of U.S. patent application Ser. No. 12/425,266 filed Apr. 16, 2009, which claims priority from U.S. Provisional Application Ser. No. 61/045,349, filed Apr. 16, 2008, which is incorporated by reference in its entirety.

### FIELD OF THE INVENTION

This invention pertains to cationic lipids, cationic lipid based drug delivery systems, ways to make them, and methods of treating diseases using them.

### BACKGROUND OF THE INVENTION

Through the development of novel delivery formulations, research is now able to focus more on improving efficacy on the therapeutic and clinical efficacious of therapeutic agents such as nucleic acids, RNA, antisense oligonucleotide, a DNA, a plasmid, a ribosomal RNA (rRNA), a micro RNA (miRNA), transfer RNA (tRNA), a small inhibitory RNA (siRNA), and small nuclear RNA (snRNA). Such novel delivery formulations will need, for example, to allow for appropriate internalization of the therapeutic agent into the cell, agents sufficient absorption from the site of administration, distribution to various tissues, sufficient residence time, concentration at the sites of action to elicit effective biologic response, while minimizing toxicity, in addition to also maintaining it's stability, and size. To this end, many efforts have been made to develop liposome or cationic polymer complexes with polyethylene glycol (PEG) or other neutral or targeting moieties. Ogris et al., Gene Ther. 6, 595-605 (1999).

However, many of the agents to date have not been found to successfully deliver therapeutic agents or to successfully deliver therapeutic agents while minimizing toxicity. As such, there is a clear need in the art to develop a novel delivery system with an improved toxicity profile as well as enhanced therapeutic agent efficacy.

### SUMMARY OF THE INVENTION

One embodiment of this invention, therefore pertains to a cationic lipid or mixtures thereof, having Formula (I)

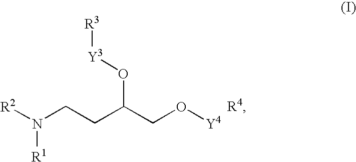

### Formula (II)

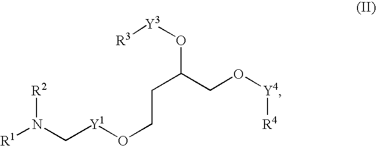

### Formula (III)

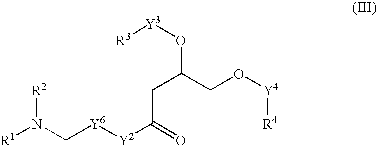

or

### Formula (IV)

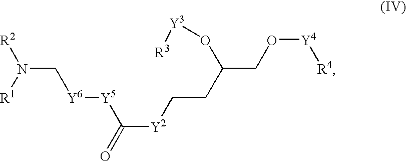

wherein Y¹ is C₁-C₆ alkylene;
Y² is CH₂, NH or O;
Y³is a bond or C(O);
Y⁴ is a bond or C(O);
Y⁵ is CH₂, NH or O;
Y⁶ is a bond or C₁-C₆ alkylene;

R¹ and R² are independently H, cycloalkyl, cycloalkenyl or R⁵; or

R¹ and R², together with the nitrogen to which they are attached, are heterocycloalkyl or heteroaryl;

one of R³ and R⁴ is H, and the other is C₁₄-C₂₀-alkenyl, or C₁₄-C₂₀-alkyl; or R³ and R⁴ are independently selected C₁₄-C₂₀-alkenyl, or C₁₄-C₂₀-alkyl; or

R³ and R⁴ together are CR²⁰R²¹, wherein R²⁰ is H and R²¹ is C₁₄-C₂₀-alkenyl, C₁₄-C₂₀-alkyl, or CH₂O—C₁₄-C₂₀-alkenyl; or R²⁰ and R²¹ are independently selected C₁₄-C₂₀-alkenyl, C₁₄-C₂₀-alkyl, or CH₂O—C₁₄-C₂₀-alkenyl;

R⁵ is alkyl, which is unsubstituted or substituted with one or more R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, C(N)NH₂, C(N)NHR⁶, C(N)N(R⁶)₂, CNOH, CNOCH₃, OH, (O), CN, N₃, NO₂, CF₃, CF₂CF₃, OCF₃, OCF₂CF₃, F, Cl, Br or I;

R⁶ is R⁷, R⁸, R⁹ or R¹⁰;

R⁷ is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁸is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁹ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁰ is alkyl, alkenyl or alkynyl;

wherein each foregoing cyclic moiety is independently unsubstituted or substituted with one or more R¹¹, OR¹¹, SR¹¹, S(O)R¹¹, SO₂R¹¹, C(O)R¹¹, CO(O)R¹¹, OC(O)R¹¹, OC(O)OR¹¹, NH₂, NHR¹¹, N(R¹¹)₂, NHC(O)R¹¹, NR¹¹C(O)R¹¹, NHS(O)₂R¹¹, NR¹¹, S(O)₂R¹¹, NHC(O)OR¹¹, NR¹¹C(O)OR¹¹, NHC(O)NH₂, NHC(O)NHR¹¹, NHC(O)N(R¹¹)₂, NR¹¹C(O)NHR¹¹, NR¹¹C(O)N(R¹¹)₂, C(O)NH₂, C(O)NHR¹¹, C(O)N(R¹¹)₂, C(O)NHOH, C(O)NHOR¹¹, C(O)NHSO₂R¹¹, C(O)NR¹¹SO₂R¹¹, SO₂NH₂, SO₂NHR¹¹, SO₂N(R¹¹)₂, C(O)H, C(O)OH, C(N)NH₂, C(N)NHR¹¹, C(N)N(R¹¹)₂, CNOH, CNOCH₃, OH, (O), CN, N₃, NO₂, CF₃, CF₂CF₃, OCF₃,OCF₂CF₃, F, Cl, Br or I;

R¹¹ is R¹², R¹³, R¹⁴, or R¹⁵.

R¹² is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹³ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁴ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁵ is alkyl, alkenyl or alkynyl, each of which is unsubstituted or substituted with one or two of independently selected R¹⁶, OR^{16E}, SR¹⁶, S(O)₂R¹⁶, C(O)OH, NH₂, NHR¹⁶ N(R¹⁶)₂, C(O)R¹⁶, C(O)NH₂, C(O)NHR¹⁶, C(O)N(R¹⁶)₂, NHC(O)R¹⁶, NR¹⁶C(O)R¹⁶, NHC(O)OR¹⁶, NR¹⁶C(O)OR¹⁶, OH, F, Cl, Br or I;

R¹⁶ is alkyl, alkenyl, alkynyl, or R¹⁷;

R¹⁷ is phenyl, heteroaryl, cycloalkyl, cycloalkenyl, heterocycloalkyl or heterocycloalkenyl;

wherein R¹², R¹³, R¹⁴, and R¹⁷ are independently unsubstituted or substituted with one or more R¹⁸, OR¹⁸, SR¹⁸, S(O)R¹⁸, SO₂R¹⁸, C(O)R¹⁸, CO(O)R¹⁸, OC(O)R¹⁸, OC(O)OR¹⁸, NH₂, NHR¹⁸, N(R¹⁸)₂, NHC(O)R¹⁸, NR¹⁸C(O)R¹⁸, NHS(O)₂R¹⁸, NR¹⁸S(O)₂R¹⁸, NHC(O)OR¹⁸, NR¹⁸C(O)OR¹⁸, NHC(O)NH₂, NHC(O)NHR¹⁸, NHC(O)N(R¹⁸)₂, NR¹⁸C(O)NHR¹⁸, NR¹⁸C(O)N(R¹⁸)₂, C(O)NH₂, C(O)NHR¹⁸, C(O)N(R¹⁸)₂, C(O)NHOH, C(O)NHOR¹⁸, C(O)NHSO₂R¹⁸, C(O)NR¹⁸SO₂R¹⁸, SO₂NH₂, SO₂NHR¹⁸, SO₂N(R¹⁸)₂, C(O)H, C(O)OH, C(N)NH₂, C(N)NHR¹⁸, C(N)N(R¹⁸)₂, CNOH, CNOCH₃, OH, (O), CN, N₃, NO₂, CF₃, CF₂CF₃, OCF₃, OCF₂CF₃, F, Cl, Br or I; and

R¹⁸ is alkyl, alkenyl, alkynyl, phenyl, heteroaryl, cycloalkyl, cycloalkenyl, heterocycloalkyl or heterocycloalkenyl.

A further embodiment pertains to Cationic-Based Lipid Encapsulation Systems (CaBLES) comprising one or more non-cationic lipids, one or more polyethylene glycol (PEG)-lipid conjugates and one or more cationic lipids having Formula I, II, III, or IV.

Another embodiment of the present invention is cationic lipids of the present invention (i.e., cationic lipids of Formula I, II, III or IV) which can be used in the preparation of either empty liposomes or used to deliver any product (e.g., therapeutic agents including nucleic acids, diagnostic agents, labels or other compounds) to a cell tissue, including cells and tissues in mammals.

In still a further embodiment, Lipid-Based Particles of the present invention are defined as CaBLES which further comprise one or more therapeutic agent(s). Such Lipid-Based Particles can be used to deliver any of a variety of therapeutic agent(s), preferably said therapeutic agent is a nucleic acid encoded with a product of interest, including but not limited to, RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), antigens, fragments thereof, proteins, peptides, vaccines and small-molecules or mixtures thereof.

A further embodiment pertains to pharmaceutical compositions comprising a Lipid-Based Particle and a pharmaceutically acceptable carrier.

A further embodiment pertains to a method of treating cancer in a mammal comprising administering thereto a therapeutically acceptable amount of a Lipid-Based Particle. Yet another embodiment pertains to a method of decreasing tumor volume in a mammal comprising administering thereto a therapeutically acceptable amount of a Lipid-Based Particle.

A further embodiment pertains to a method of making Lipid-Based Particles, comprising: (a) mixing the cationic lipid(s), the non-cationic lipid(s) and the PEG-lipid conjugate(s); (b) adding the mixture of step (a) to one or more therapeutic agents; and (c) separating and purifying resulting suspension of step (b).

### DESCRIPTION OF THE DRAWINGS

FIGS. 1-48. In vivo delivery and vitro transfection activity of selected cationic lipids that were formulated as disclosed herein.

FIG. 49. In vivo activity of targeted Lipid-Based Particles with two levels of PEG-loading versus NTC compositions.

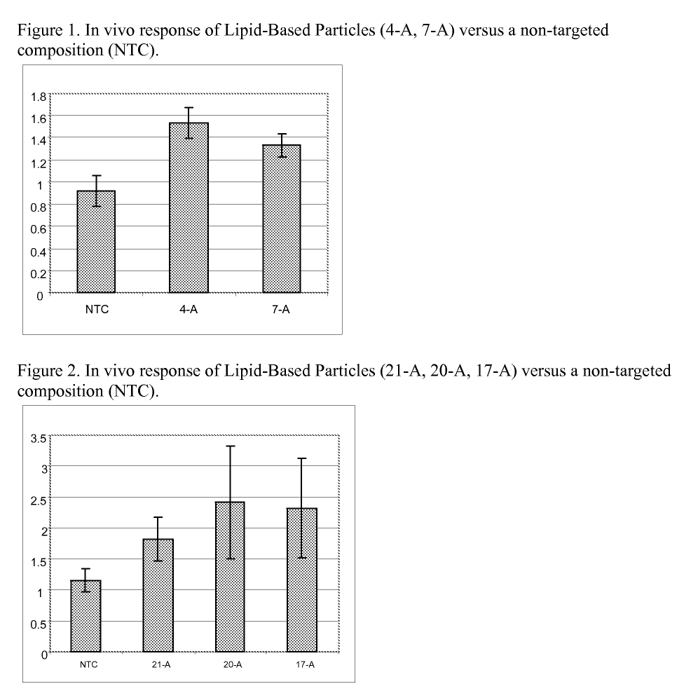

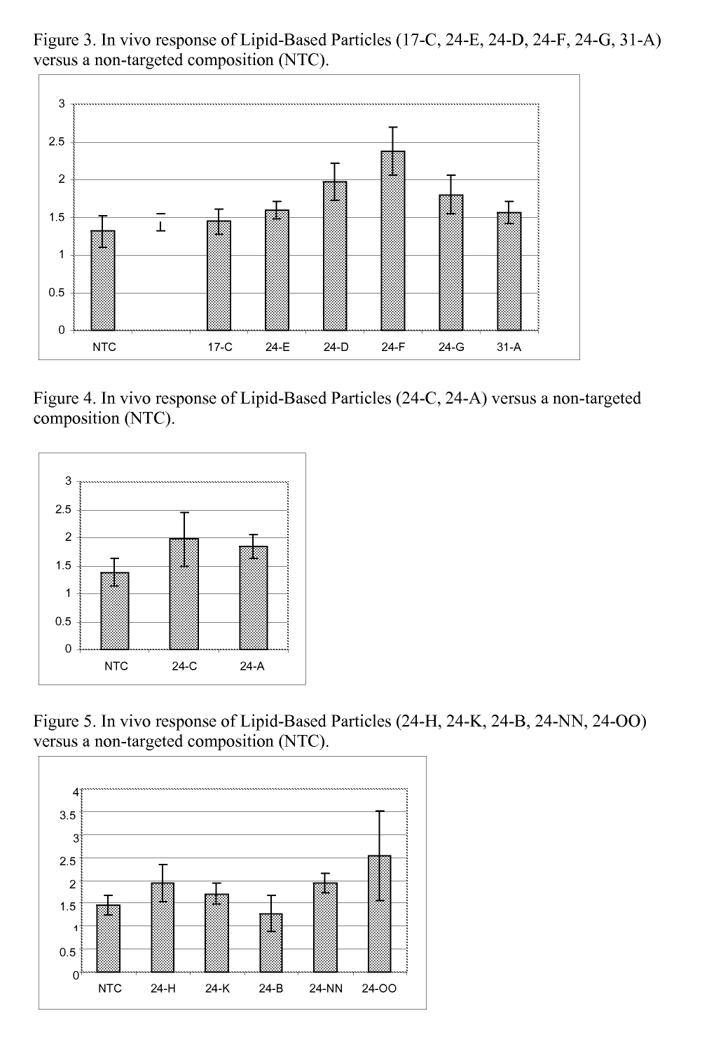

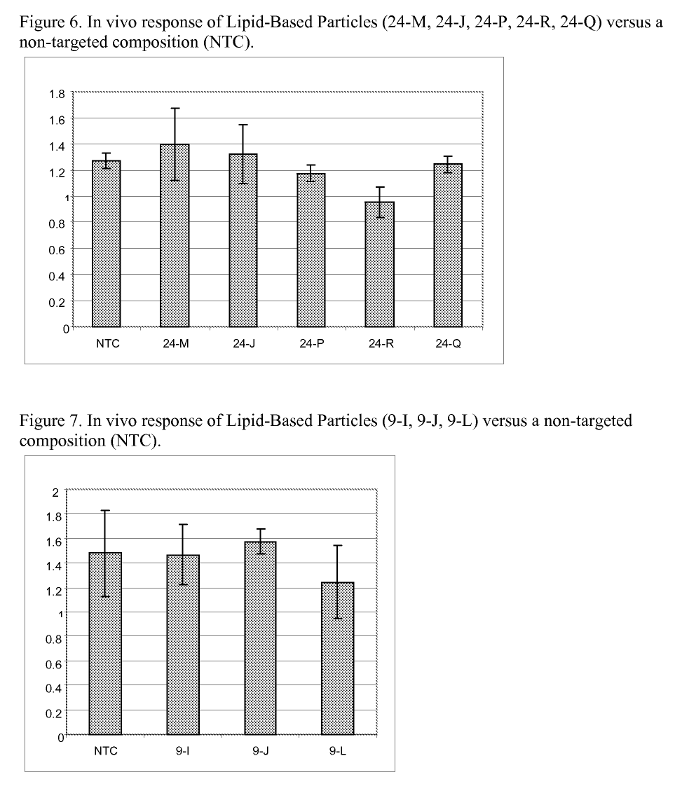

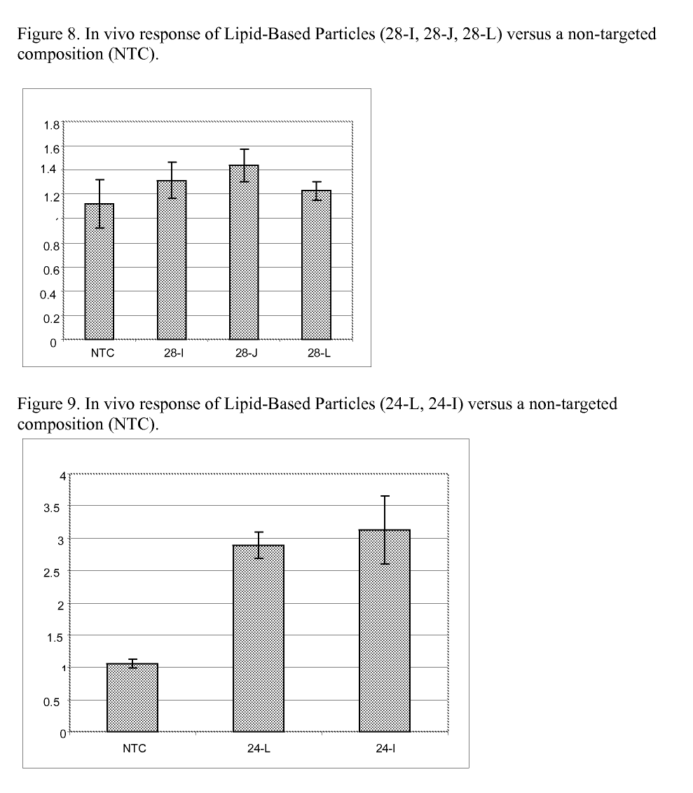

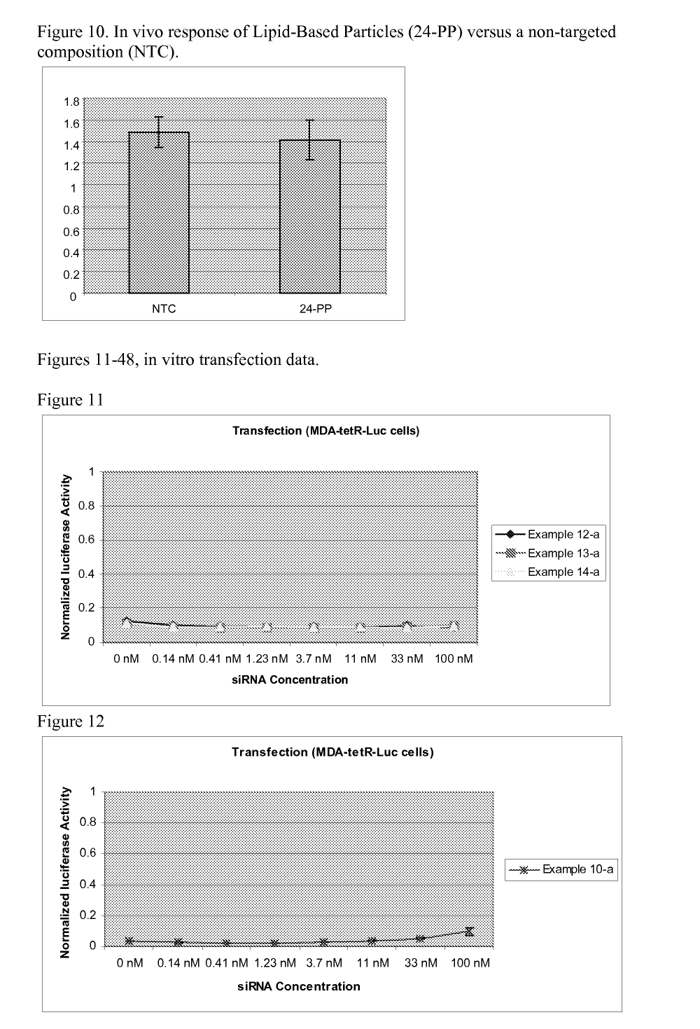

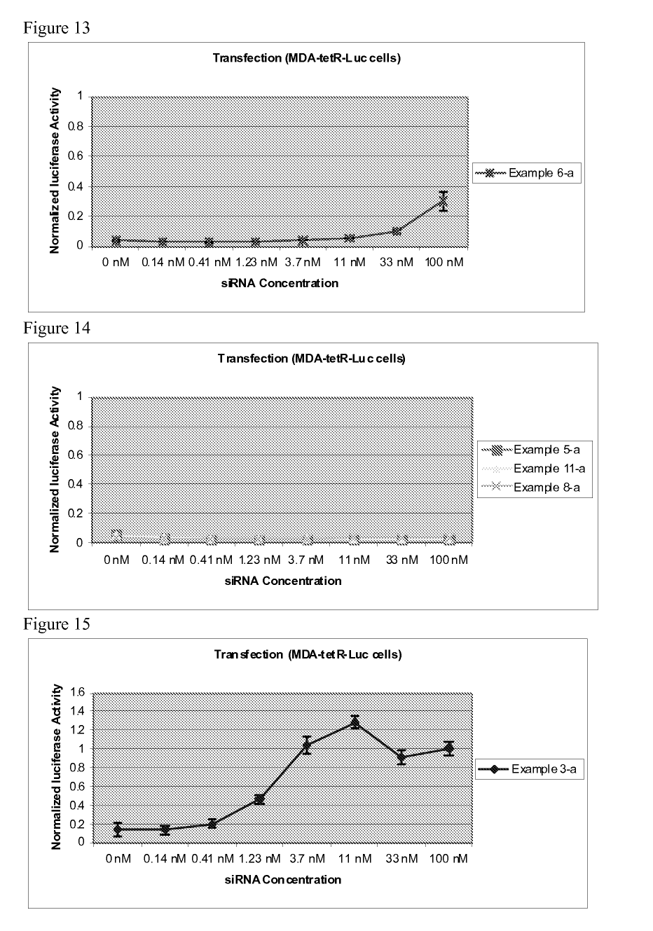

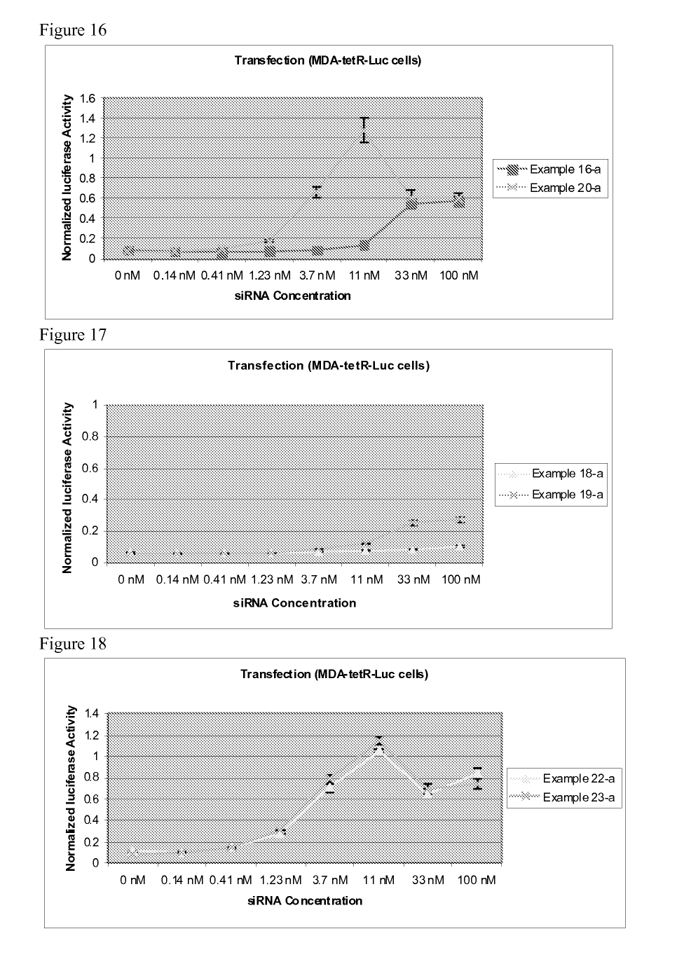

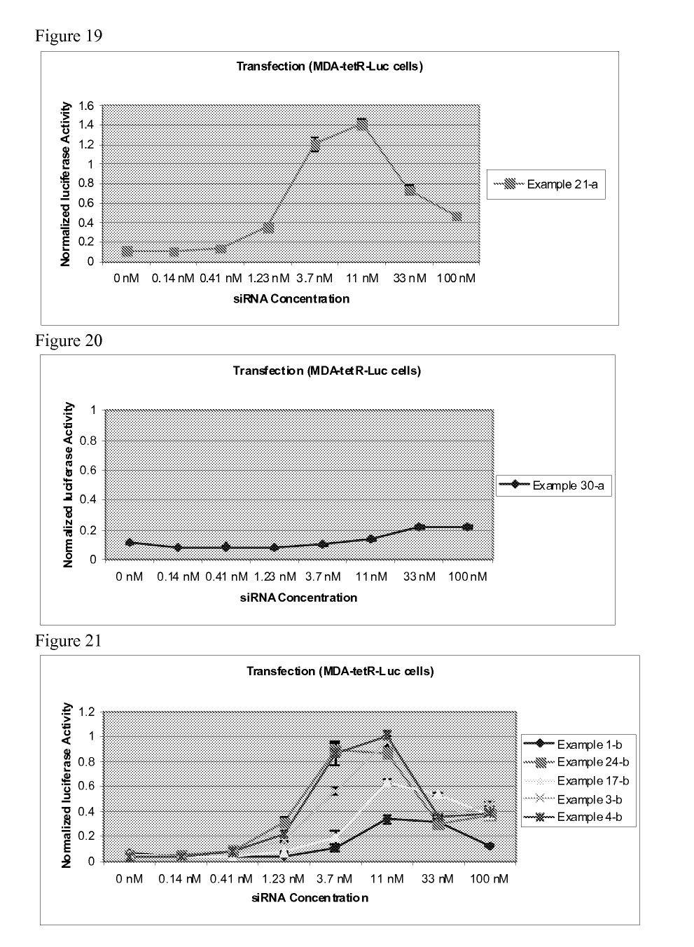

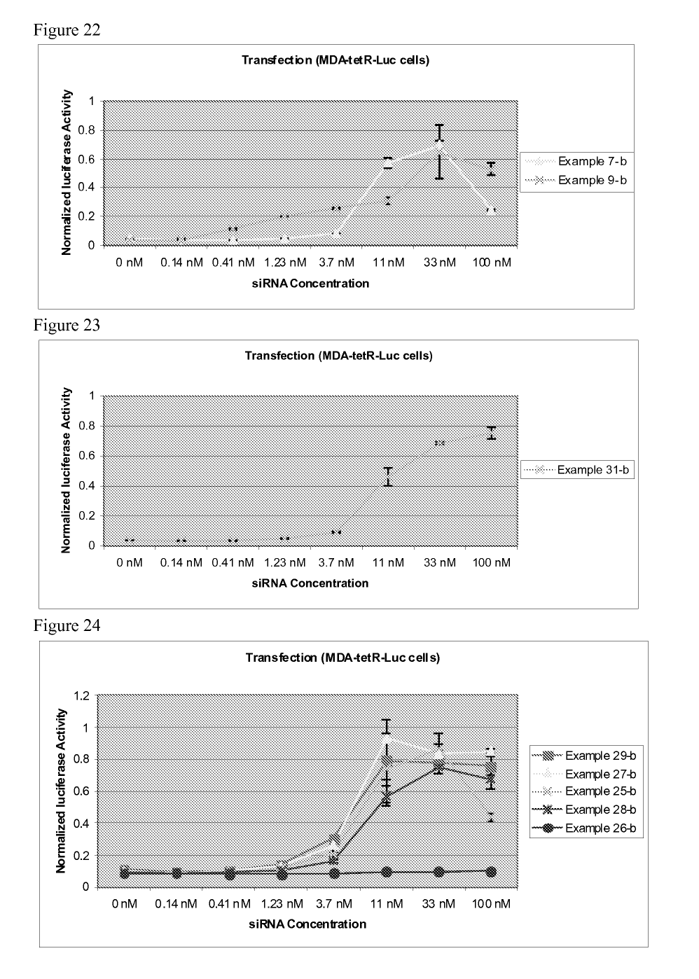

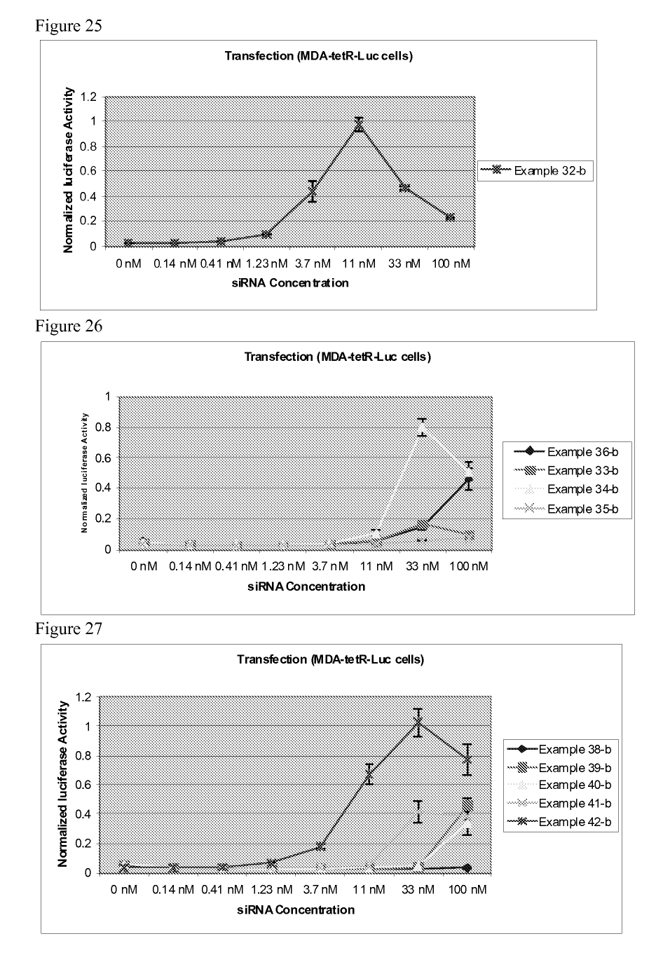

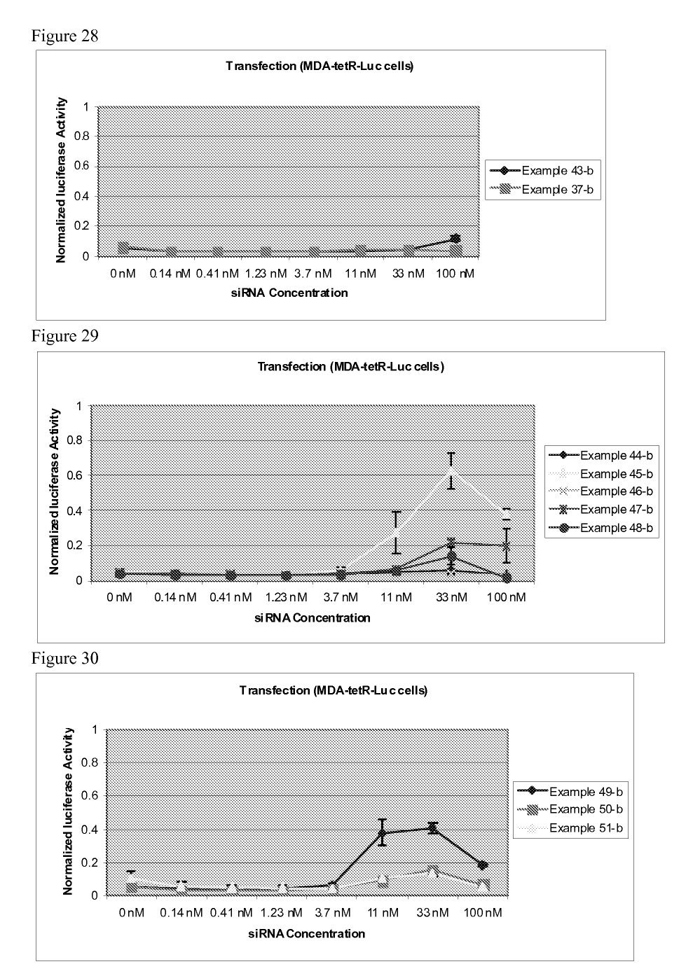

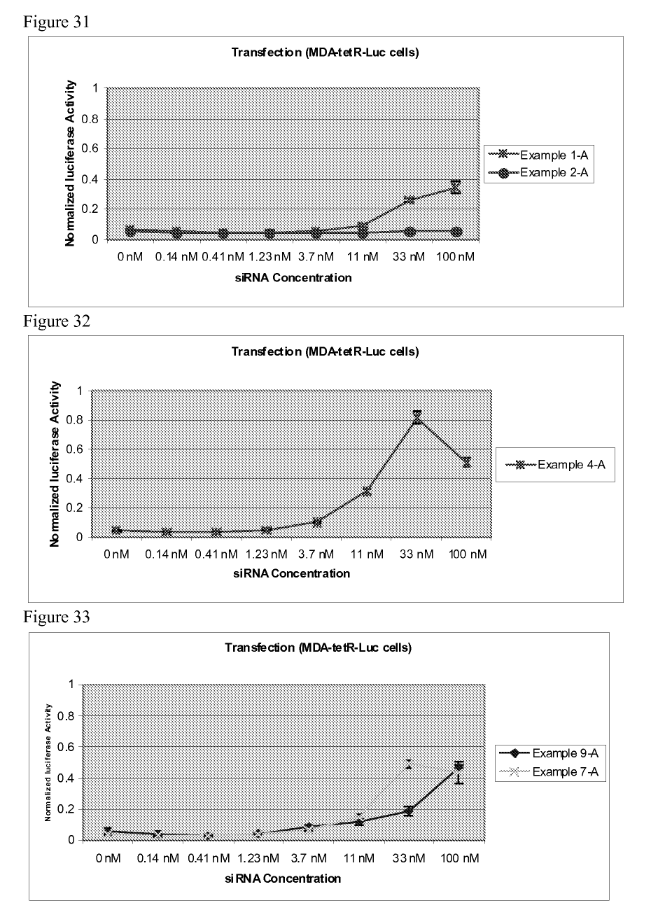

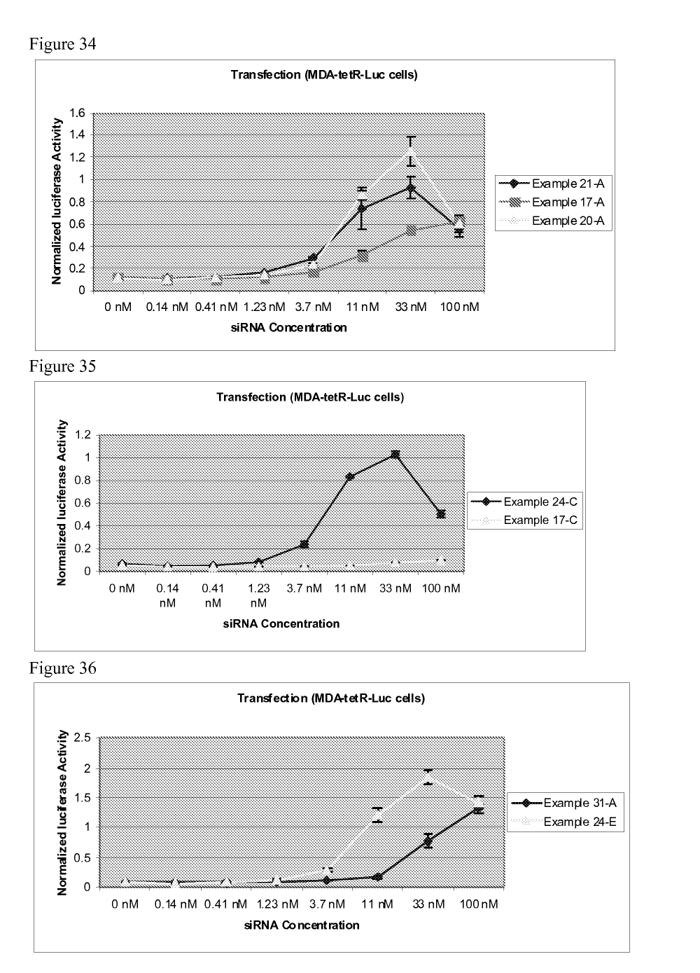

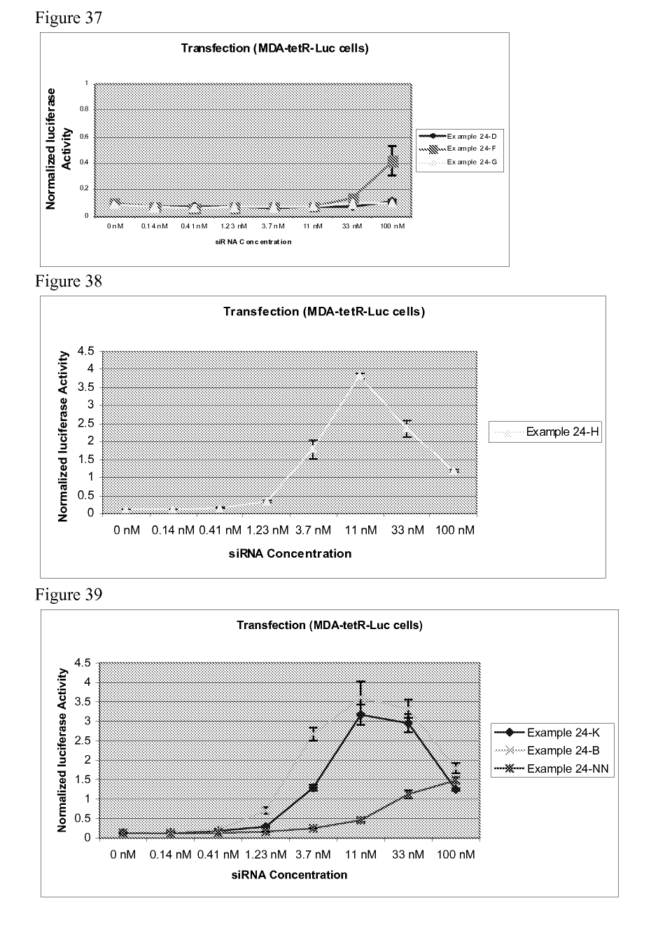

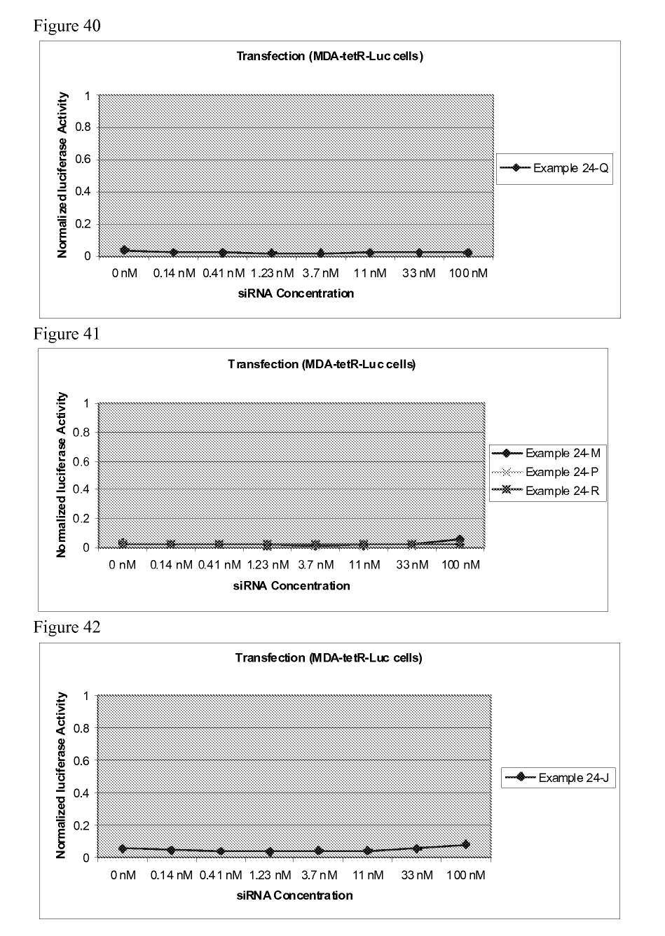

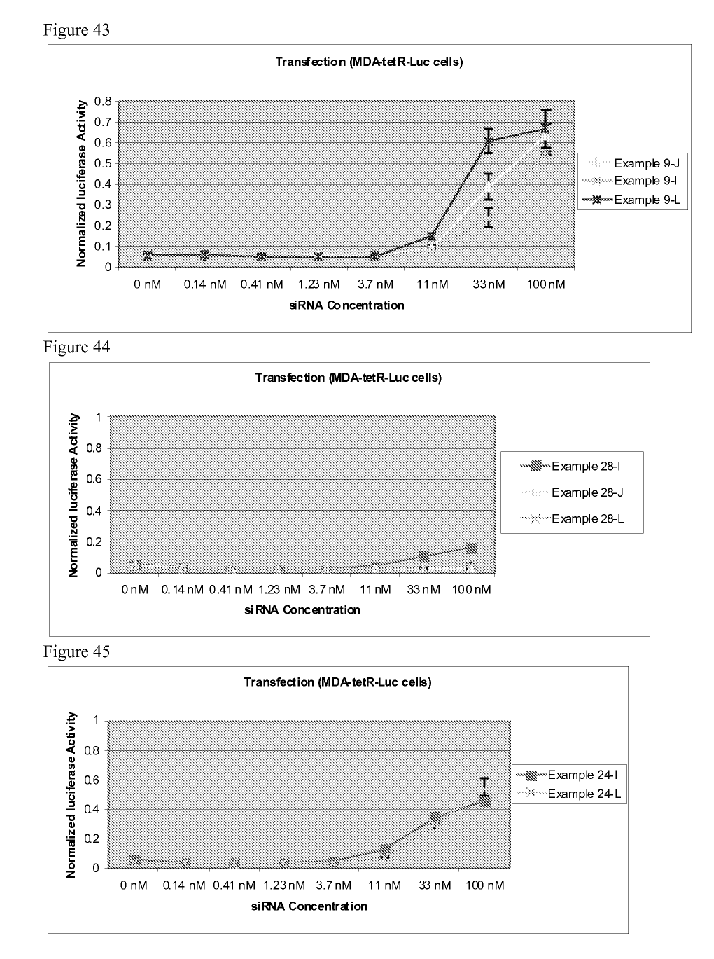

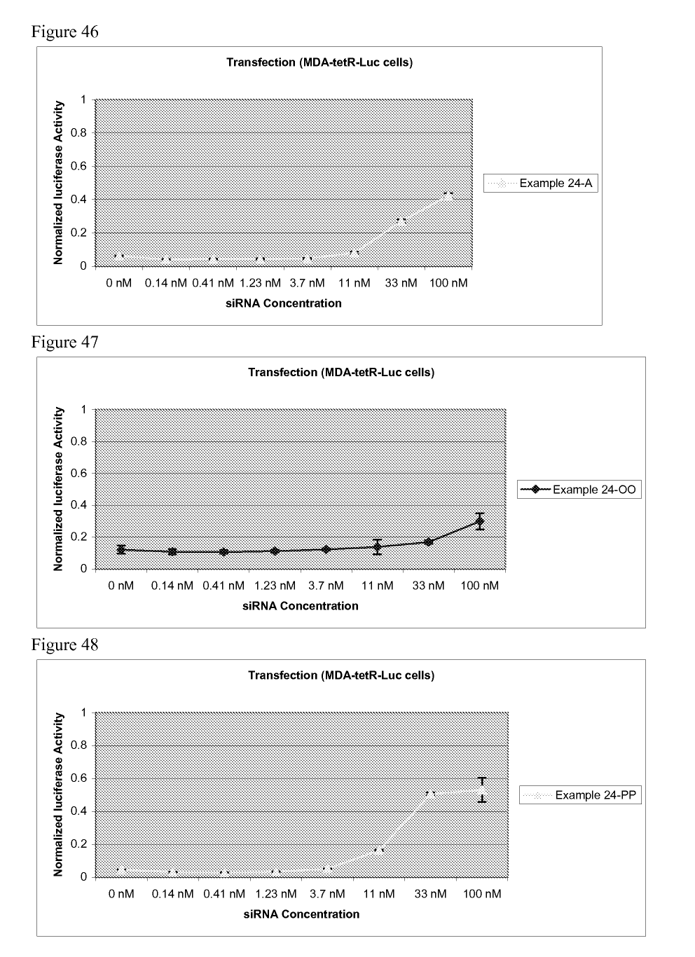

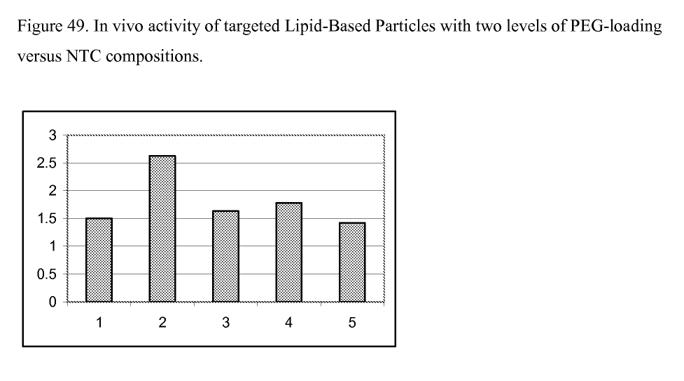

### DETAILED DESCRIPTION OF THE INVENTION

This invention pertains to in vitro and in vivo delivery of therapeutic agents. In particular, the invention pertains to compositions that allow for delivery of nucleic acids, including but not limited to RNA, antisense oligonucleotide, a DNA, a plasmid, a ribosomal RNA (rRNA), a micro RNA (miRNA), transfer RNA (tRNA), a small inhibitory RNA (siRNA), small nuclear RNA(snRNA), antigens, fragments thereof, proteins, peptides, and small molecules.

Variable moieties of compounds herein are represented by identifiers (capital letters with numerical and/or alphabetical superscripts) and may be specifically embodied.

It is also meant to be understood that a specific embodiment of a variable moiety may be the same or different as another specific embodiment having the same identifier and that asymmetric divalent moieties are drawn from left to right.

As used in the specification and the appended claims, unless specified to the contrary, the following terms have the meaning indicated:

The term “alkenyl,” as used herein, means monovalent, straight or branched chain hydrocarbon moieties having one or more than one carbon-carbon double bonds, such as C₂-alkenyl, C₃-alkenyl, C₄-alkenyl, C₅-alkenyl, C₆-alkenyl and the like.

The term “C₁-C₆-alkylene,” as used herein, means divalent, saturated, straight or branched chain hydrocarbon moieties bonds, such as C₁-alkylene, C₂-alkylene, C₃-alkylene, C₄-alkylene, C₅-alkylene and C₆-alkylene.

The terms “alkyl,” as used herein, means monovalent, straight or branched chain hydrocarbon moieties such as C₁-alkyl, C₂-alkyl, C₃-alkyl, C₄-alkyl, C₅-alkyl and C₆-alkyl.

The term “alkynyl,” as used herein, means monovalent, straight or branched chain hydrocarbon moieties having one or more than one carbon-carbon triple bonds, such as C₂-alkynyl, C₃-alkynyl, C₄-alkynyl, C₅-alkynyl, C₆-alkynyl and the like.

The term “C₁-C₈-alkyl” as used herein, means C₁-alkyl, C₂-alkyl, C₃-alkyl, C₄-alkyl, C₅-alkyl, C₆-alkyl, C₇-alkyl and C₈-alkyl.

The term “C₁₄-C₂₀-alkenyl,” as used herein, means C₁₄-alkenyl,” C₁₅-alkenyl,” C₁₆-alkenyl,” C₁₇-alkenyl,” C₁₈-alkenyl,” C₁₉-alkenyl” and C₂₀-alkenyl.”

The term “C₁₄-C₂₀-alkyl,” as used herein, means C₁₄-alkyl,” C₁₅-alkyl,” C₁₆-alkyl,”C₁₇-alkyl,” C₁₈-alkyl,” C₁₉-alkyl” and C₂₀-alkyl.”

The term “cycloalkane,” as used herein, means saturated cyclic or bicyclic hydrocarbon moieties, such as C₃-cycloalkane, C₄-cycloalkane, C₅-cycloalkane, C₆-cycloalkane and the like.

The term “cycloalkyl,” as used herein, means monovalent, saturated cyclic and bicyclic hydrocarbon moieties, such as C₃-cycloalkyl, C₄-cycloalkyl, C₅-cycloalkyl, C₆-cycloalkyl and the like.

The term “cycloalkene,” as used herein, means cyclic and bicyclic hydrocarbon moieties having one or more than one carbon-carbon double bonds, such as C₅-cycloalkene, C₆-cycloalkene and the like.

The term “cycloalkenyl,” as used herein, means monovalent, cyclic hydrocarbon moieties having one or more than one carbon-carbon double bonds, such as C₄-cycloalkenyl, C₅-cycloalkenyl, C₆-cycloalkenyl and the like.

The term “heteroarene,” as used herein, means a five-membered or six-membered aromatic ring having at least one carbon atom and one or more than one independently selected nitrogen, oxygen or sulfur atom. The heteroarenes of this invention are connected through any adjacent atoms in the ring, provided that proper valences are maintained. Examples of heteroarenes include, but are not limited to furan, imidazole, isothiazole, isoxazole, oxadiazole, oxazole, pyrazine, pyrazole, pyridazine, pyridine, pyrimidine, pyrrole, thiazole, thiadiazole thiophene, tetrazine, tetrazole, triazine, triazole and the like.

The term “heteroaryl,” as used herein, means a monovalent five-membered or six-membered aromatic ring having at least one carbon atom and one or more than one independently selected nitrogen, oxygen or sulfur atom. The heteroaryls of this invention are connected through any carbon atom or any nitrogen atom in the ring, provided that proper valences are maintained. Examples of heteroaryls include, but are not limited to, furanyl, imidazolyl, isothiazolyl, isoxazolyl, oxadiazolyl, oxazolyl, pyrazinyl, pyrazolyl, pyridazinyl, pyridinyl, pyrimidinyl, pyrrolyl, tetrazolyl, thiadiazolyl, thiazolyl, thienyl, triazinyl, triazolyl and the like.

The term “heterocycloalkane,” as used herein, means cycloalkane having one or two or three CH₂ moieties replaced with independently selected O, S, S(O), SO₂ or NH and one or two CH moieties unreplaced or replaced with N and also means cycloalkane having one or two or three CH₂ moieties unreplaced or replaced with independently selected O, S, S(O), SO₂ or NH and one or two CH moieties replaced with N.

The term “heterocycloalkene,” as used herein, means cycloalkene having one or two or three CH₂ moieties replaced with independently selected O, S, S(O), SO₂ or NH and one or two CH moieties unreplaced or replaced with N and also means cycloalkene having one or two or three CH₂ moieties unreplaced or replaced with independently selected O, S, S(O), SO₂ or NH and one or two CH moieties replaced with N.

The term “heterocycloalkyl,” as used herein, means cycloalkyl having one or two or three CH₂ moieties replaced with independently selected O, S, S(O), SO₂ or NH and one or two CH moieties unreplaced or replaced with N and also means cycloalkyl having one or two or three CH₂ moieties unreplaced or replaced with independently selected O, S, S(O), SO₂ or NH and one or two CH moieties replaced with N.

The term “heterocycloalkenyl,” as used herein, means cycloalkenyl having one or two or three CH₂ moieties replaced with independently selected O, S, S(O), SO₂ or NH and one or two CH moieties unreplaced or replaced with N and also means cycloalkenyl having one or two or three CH₂ moieties unreplaced or replaced with independently selected O, S, S(O), SO₂ or NH and one or two CH moieties replaced with N.

The term “cyclic moiety,” as used herein, means benzene, cycloalkane, cycloalkyl, cycloalkene, cycloalkenyl, heteroarene, heteroaryl, heterocycloalkane, heterocycloalkyl, heterocycloalkene, heterocycloalkenyl and phenyl.

The term “DSPC,” as used herein, means 1,2-distearoyl-sn-glycero-3-phosphocholine.

The term, “Chol,” as used herein, means cholesterol.

The term, “PEG-Chol,” as used herein, means poly(oxy-1,2-ethanediyl)-2000-α-(3β)-cholest-5-en-3-yl-omega-hydroxy.

The term, “Pal-PEG-Cera,” as used herein, means N-palmitoyl-sphingosine-1-[succinyl(methoxypolyethylene glycol)-2000].

The term, “PEG-DMPE,” as used herein, means N-(carbonyl-methoxypolyethyleneglycol-2000)-1,2-dimyristoyl-sn-glycero-3-phosphoethanolamine.

The term, “PEG-DPPE,” as used herein, means N-(carbonyl-methoxypolyethyleneglycol-2000)-1,2-dipalmitoyl-sn-glycero-3-phosphoethanolamine.

The term, “PEG-DSPE,” as used herein, means N-(carbonyl-methoxypolyethyleneglycol-2000)-1,2-distearoyl-sn-glycero-3-phosphoethanolamine.

The term, “PEG-DMG,” as used herein, means 1,2-dimyristoyl-sn-glycerol-methoxypolyethyleneglycol-2000.

The term, “PEG-DPG,” as used herein, means 1,2-dipalmitoyl-sn-glycerol-methoxypolyethyleneglycol-2000.

The term, “PEG-DSG,” as used herein, means 1,2-distearoyl-sn-glycerol-methoxypolyethyleneglycol-2000.

The term, “SPC,” as used herein, means soybean phosphatidylcholine.

The term “MALDI,” as used herein, means matrix assisted laser desorption ionization.

The term, “particle,” as used herein, means a small object that behaves as a whole unit in terms of its transport and properties.

The term, “nanoparticle,” as used herein, means any particle having a diameter of less than 1000 nanometers. In some embodiments, nanoparticles have a diameter of 500 or less. In some embodiments, nanoparticles have a diameter of 200 or less.

The term “nucleic acid” or “polynucleotide” refers to a polymer containing at least two deoxyribonucleotides or ribonucleotides in either single- or double-stranded form. Nucleic acids include nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless specifically limited, the terms encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). “Nucleotides” contain a sugar deoxyribose (DNA) or ribose (RNA), a base, and a phosphate group. Nucleotides are linked together through the phosphate groups. Nucleotides include chemically modified nucleotides as described in, e.g., WO 03/74654. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. DNA may be in the form of antisense, plasmid DNA, parts of a plasmid DNA, pre-condensed DNA, product of a polymerase chain reaction (PCR), vectors (P1, PAC, BAC, YAC, artificial chromosomes), expression cassettes, chimeric sequences, chromosomal DNA, or derivatives of these groups. The term nucleic acid is used interchangeably with gene, plasmid, cDNA, mRNA, and an interfering RNA molecule (e.g. a synthesized siRNA or an siRNA expressed from a plasmid).

The term, “siRNA,” as used herein means a small inhibitory RNA, and molecules having endogenous RNA bases or chemically modified nucleotides. The modifications shall not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides or a combination thereof.

The term “small molecule,” as used herein, means antibiotics, antineoplastics, antiinflammatories, anitivirals, immunomodulators and agents that act upon the respiratory system, the cardiovascular system, the central nervous system or a metabolic pathway involved with dyslipidemia, diabetes or Syndrome X.

The term, “NTC,” as used herein, means a non-targeted composition containing one or more (PEG)-lipid conjugates, one or more non-cationic lipids, one or more cationic lipids, and one or more non-targeted agents such as a non-targeted siRNA (sequence: UGGUUUACAUGUUGUGUGA SEQ ID NO: 3).

### Compounds

Compounds of this invention may contain asymmetrically substituted carbon atoms in the R or S configuration, wherein the terms “R” and “S” are as defined in Pure Appl. Chem. (1976) 45, 13-10. Compounds having asymmetrically substituted carbon atoms with equal amounts of R and S configurations are racemic at those atoms. Atoms having excess of one configuration over the other are assigned the configuration in excess, preferably an excess of about 85%-90%, more preferably an excess of about 95%-99%, and still more preferably an excess greater than about 99%. Accordingly, this invention is meant to embrace racemic mixtures and relative and absolute diastereoisomers and the compounds thereof.

Compounds of this invention may also contain carbon-carbon double bonds or carbon-nitrogen double bonds in the E or Z configuration, wherein the term “E” represents higher order substituents on opposite sides of the carbon-carbon or carbon-nitrogen double bond and the term “Z” represents higher order substituents on the same side of the carbon-carbon or carbon-nitrogen double bond as determined by the Cahn-Ingold-Prelog Priority Rules. The compounds of this invention may also exist as a mixture of “E” and “Z” isomers.

Compounds of this invention can exist in an isotopic form containing one or more atoms having an atomic mass or mass number different from the atomic mass or mass number most abundantly found in nature. Isotopes of atoms such as hydrogen, carbon, phosphorous, sulfur fluorine, chlorine, and iodine include, but are not limited to, ²H, ³H, ¹⁴C, ³²P, ³⁵S, ¹⁸F, ³⁶Cl, and ¹²⁵I, respectively. Compounds that contain other isotopes of these and/or other atoms are within the scope of this invention. Compounds containing tritium (³H) and ¹⁴C radioisotopes are preferred in general for their ease in preparation and detectability for radiolabeled compounds. Isotopically labeled compounds of this invention can be prepared by the general methods well known to persons having ordinary skill in the art. Such isotopically labeled compounds can be conveniently prepared by carrying out the procedures disclosed in the Examples and Schemes herein by substituting a readily available isotopically labeled reagent for a non-isotopically labeled reagent.

Suitable groups for Y¹, Y², Y³, Y⁴, Y⁵, Y⁶, R¹, R², R³, and R⁴ in compounds of Formula (I), (II), (III), and (IV) are independently selected. The described embodiments of the present invention may be combined. Such combination is contemplated and within the scope of the present invention. For example, it is contemplated that embodiments for any of Y¹, Y², Y³, Y⁴, Y⁵, Y⁶, R¹, R², R³, and R⁴ can be combined with embodiments defined for any other of Y¹, Y², Y³, Y⁴, Y⁵, Y⁶, R¹, R², R³ and R⁴.

One embodiment of this invention, therefore pertains to a cationic lipid or mixtures thereof, having Formula (I)

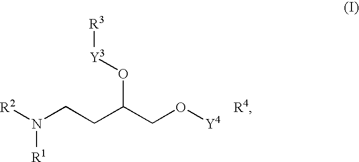

### Formula (II)

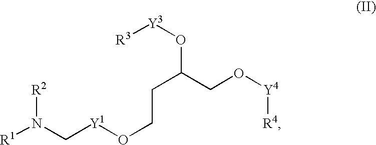

### Formula (III)

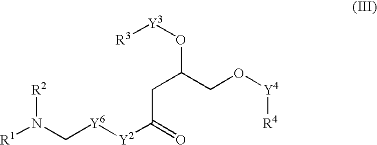

or

### Formula (IV)

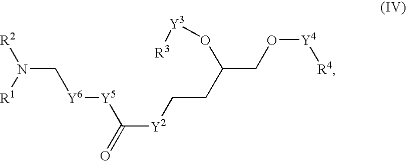

wherein Y¹ is C₁-C₆ alkylene;

Y² is CH₂, NH or O;

Y³ is a bond or C(O);

Y⁴ is a bond or C(O);

Y⁵ is CH₂, NH or O;

Y⁶ is a bond or C₁-C₆ alkylene;

R¹ and R² are independently H, cycloalkyl, cycloalkenyl or R⁵; or

R¹ and R², together with the nitrogen to which they are attached, are heterocycloalkyl or heteroaryl;

one of R³ and R⁴ is H, and the other is C₁₄-C₂₀-alkenyl, or C₁₄-C₂₀-alkyl; or R³ and R⁴ are independently selected C₁₄-C₂₀-alkenyl, or C₁₄-C₂₀-alkyl; or

R³ and R⁴ together are CR²⁰R²¹, wherein R²⁰ is H and R²¹ is C₁₄-C₂₀-alkenyl, C₁₄-C₂₀-alkyl, or CH₂O—C₁₄-C₂₀-alkenyl; or R²⁰ and R²¹ are independently selected C₁₄-C₂₀-alkenyl, C₁₄-C₂₀-alkyl, or CH₂O—C₁₄-C₂₀-alkenyl;

R⁵ is alkyl, which is unsubstituted or substituted with one or more R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, C(N)NH₂, C(N)NHR⁶, C(N)N(R⁶)₂, CNOH, CNOCH₃, OH, (O), CN, N₃, NO₂, CF₃, CF₂CF₃, OCF₃, OCF₂CF₃, F, Cl, Br or I;

R⁶ is R⁷, R⁸, R⁹, or R¹⁰;

R⁷ is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁸ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁹ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁰ is alkyl, alkenyl or alkynyl;

wherein each foregoing cyclic moiety is independently unsubstituted or substituted with one or more R¹¹OR¹¹, SR¹¹, S(O)R¹¹, SO₂R¹¹, C(O)R¹¹, CO(O)R¹¹, OC(O)R¹¹, OC(O)OR¹¹, NH₂, NHR¹¹, N(R¹¹)₂, NHC(O)R¹¹, NR¹¹C(O)R¹¹, NHS(O)₂R¹¹, NR¹¹S(O)₂R¹¹, NHC(O)OR¹¹, NR¹¹C(O)OR¹¹, NHC(O)NH₂, NHC(O)NHR¹¹, NHC(O)N(R¹¹)₂, NR¹¹C(O)NHR¹¹, NR¹¹C(O)N(R¹¹)₂, C(O)NH₂, C(O)NHR¹¹, C(O)N(R¹¹)₂, C(O)NHOH, C(O)NHOR¹¹, C(O)NHSO₂R¹¹, C(O)NR¹¹SO₂R¹¹, SO₂NH₂, SO₂NHR¹¹, SO₂N(R¹¹)₂, C(O)H, C(O)OH, C(N)NH₂, C(N)NHR¹¹, C(N)N(R¹¹)₂, CNOH, CNOCH₃, OH, (O), CN, N₃, NO₂, CF₃, CF₂CF₃, OCF₃, OCF₂CF₃, F, Cl, Br or I;

R¹¹ is R¹², R¹³, R¹⁴, or R¹⁵;

R¹² is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹³ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁴ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁵ is alkyl, alkenyl or alkynyl, each of which is unsubstituted or substituted with one or two of independently selected R¹⁶, OR^{16E}, SR¹⁶, S(O)₂R¹⁶, C(O)OH, NH₂, NHR¹⁶ N(R¹⁶)₂, C(O)R¹⁶, C(O)NH₂, C(O)NHR¹⁶, C(O)N(R¹⁶)₂, NHC(O)R¹⁶, NR¹⁶C(O)R¹⁶, NHC(O)OR¹⁶, NR¹⁶C(O)OR¹⁶, OH, F, Cl, Br or I;

R¹⁶ is alkyl, alkenyl, alkynyl, or R¹⁷;

R¹⁷ is phenyl, heteroaryl, cycloalkyl, cycloalkenyl, heterocycloalkyl or heterocycloalkenyl;

wherein R¹², R¹³, R¹⁴ and R¹⁷ are independently unsubstituted or substituted with one or more R¹⁸, OR¹⁸, SR¹⁸, S(O)R¹⁸, SO₂R¹⁸, C(O)R¹⁸, CO(O)R¹⁸, OC(O)R¹⁸, OC(O)OR¹⁸, NH₂, NHR¹⁸, N(R¹⁸)₂, NHC(O)R¹⁸, NR¹⁸C(O)R¹⁸, NHS(O)₂R¹⁸, NR¹⁸S(O)₂R¹⁸, NHC(O)OR¹⁸, NR¹⁸C(O)OR¹⁸, NHC(O)NH₂, NHC(O)NHR¹⁸, NHC(O)N(R¹⁸)₂, NR¹⁸C(O)NHR¹⁸, NR¹⁸C(O)N(R¹⁸)₂, C(O)NH₂, C(O)NHR¹⁸, C(O)N(R¹⁸)₂, C(O)NHOH, C(O)NHOR¹⁸, C(O)NHSO₂R¹⁸, C(O)NR¹⁸SO₂R¹⁸, SO₂NH₂, SO₂NHR¹⁸, SO₂N(R¹⁸)₂, C(O)H, C(O)OH, C(N)NH₂, C(N)NHR¹⁸, C(N)N(R¹⁸)₂, CNOH, CNOCH₃, OH, (O), CN, N₃, NO₂, CF₃, CF₂CF₃, OCF₃, OCF₂CF₃, F, Cl, Br or I; and

R¹⁸ is alkyl, alkenyl, alkynyl, phenyl, heteroaryl, cycloalkyl, cycloalkenyl, heterocycloalkyl or heterocycloalkenyl.

Another embodiment of this invention, therefore pertains to a cationic lipid or mixtures thereof, having Formula (I)

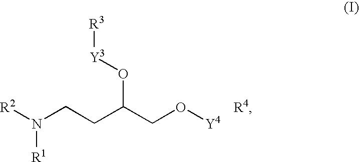

### Formula (II)

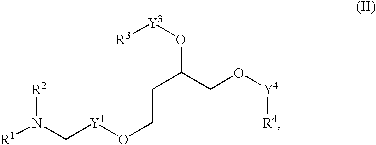

### Formula (III)

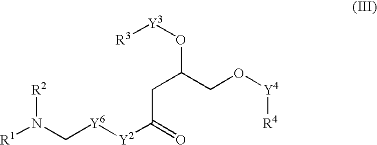

or

### Formula (IV)

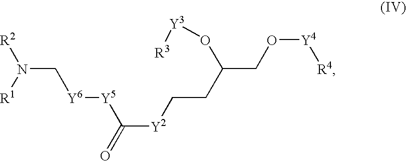

wherein Y¹ is C₁-C₆ alkylene;

Y² is NH or O;

Y³ is a bond or C(O);

Y⁴ is a bond or C(O);

Y⁵ is CH₂, or NH;

Y⁶ is a bond or C₁-C₆ alkylene;

R¹ and R² are independently H or R⁵; or

R¹ and R², together with the nitrogen to which they are attached, are heterocycloalkyl; or

R³ and R⁴ are independently selected C₁₄-C₂₀-alkenyl, or C₁₄-C₂₀-alkyl; or

R³ and R⁴ together are CR²⁰R²¹, wherein R²⁰ and R²¹ are independently selected C₁₄-C₂₀-alkenyl;

R⁵ is alkyl, which is unsubstituted or substituted with one or more R⁶, OR⁶, or N(R⁶)₂;

R⁶ is R⁷, R⁸, R⁹, or R¹⁰;

R⁷ is phenyl;

R⁸ is heteroaryl;

R⁹ is heterocycloalkyl;

R¹⁰ is alkyl;

wherein each foregoing cyclic moiety is independently unsubstituted or substituted with one or more R¹¹, OR¹¹, N(R¹¹)₂, or F, Cl, Br or I;

R¹¹ is R¹², R¹³, or R¹⁵;

R¹² is phenyl;

R¹³ is heteroaryl;

R¹⁵ is alkyl;

wherein R¹² and R¹³ are independently unsubstituted or substituted with OR¹⁸; and

R¹⁸ is alkyl.

One embodiment of this invention, therefore pertains to a cationic lipid or mixtures thereof, having Formula (I)

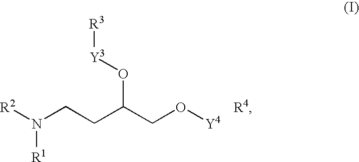

wherein Y³ and Y⁴ are each a bond. Another embodiment of Formula (I) pertains to compounds wherein Y³ and Y⁴ are each C(O). Another embodiment of Formula (I) pertains to compounds wherein Y³ is a bond and Y⁴ is C(O). Another embodiment of Formula (I) pertains to compounds wherein Y⁴ is a bond and Y³ is C(O).

One embodiment of Formula (I) pertains to compounds wherein R¹ and R² are each R⁵. Another embodiment of Formula (I) pertains to compounds wherein R¹ is H and R²is R⁵. Another embodiment of Formula (I) pertains to compounds wherein R¹ and R² together with the nitrogen to which they are attached, are heterocycloalkyl. Another embodiment of Formula (I) pertains to compounds wherein R¹ and R² together with the nitrogen to which they are attached, are heteroaryl. Another embodiment of Formula (I) pertains to compounds wherein R¹ and R² together with the nitrogen to which they are attached, are pyrrolidinyl, piperidinyl, morpholinyl, or piperazinyl. Another embodiment of Formula (I) pertains to compounds wherein R¹ and R² together with the nitrogen to which they are attached, are pyrrolidinyl.

One embodiment of Formula (I) pertains to compounds wherein R³ and R⁴ are C₁₄-C₂₀-alkenyl. Another embodiment of Formula (I) pertains to compounds wherein R³ and R⁴ are C₁₄-C₂₀-alkyl. Another embodiment of Formula (I) pertains to compounds wherein R³ and R⁴ together are CR²⁰R²¹, wherein R²⁰ and R²¹ are each C₁₄-C₂₀-alkenyl. Another embodiment of Formula (I) pertains to compounds wherein R³ is H; and R⁴ is C₁₄-C₂₀-alkenyl.

One embodiment of Formula (I) pertains to compounds wherein R⁵is alkyl. Another embodiment of Formula (I) pertains to compounds wherein R⁵is alkyl which is unsubstituted. Another embodiment of Formula (I) pertains to compounds wherein R⁵is alkyl which is substituted. Another embodiment of Formula (I) pertains to compounds wherein R⁵is alkyl which is substituted with R⁶, OR⁶, or N(R⁶)₂.

One embodiment of Formula (I) pertains to compounds wherein R⁶ is R⁷; and R⁷ is phenyl which is unfused. Another embodiment of Formula (I) pertains to compounds wherein R⁶ is R⁸; and R⁸ is heteroaryl, which is unfused. Another embodiment of Formula (I) pertains to compounds wherein R⁶ is R⁹; and R⁹ is heterocycloalkyl, which is unfused. Another embodiment of Formula (I) pertains to compounds wherein R⁶ is R¹⁰; and R¹⁰ is alkyl, which is unsubstituted.

One embodiment of Formula (I) pertains to compounds wherein all foregoing cyclic moieties are unsubstituted. Another embodiment of Formula (I) pertains to compounds wherein one or more cyclic moieties are substituted. Another embodiment of Formula (I) pertains to compounds wherein one or more cyclic moieties are substituted with one or more R¹¹, OR¹¹, or N(R¹¹)₂, or F.

One embodiment pertains to compounds of Formula (I) wherein Y³ and Y⁴ are each a bond; R¹ and R² together with the nitrogen to which they are attached, are heterocycloalkyl; and R³ and R⁴ are C₁₄-C₂₀-alkenyl. Another embodiment of Formula (I) pertains to compounds wherein Y³ and Y⁴ are each a bond; R¹ and R² are each independently R⁵; R⁵ is alkyl which is unsubstituted; and R³ and R⁴ are each C₁₄-C₂₀-alkenyl. Another embodiment of Formula (I) pertains to compounds wherein Y³ and Y⁴ are each a bond; R¹ and R² together with the nitrogen to which they are attached, are heterocycloalkyl; R³ and R⁴ are C₁₄-C₂₀-alkenyl; wherein the heterocycloalkyl is substituted with R¹¹; R¹¹ is R¹²; and R¹² is phenyl which is unfused. Another embodiment of Formula (I) pertains to compounds wherein Y³ and Y⁴ are each a bond; R¹ and R² together with the nitrogen to which they are attached, are heterocycloalkyl; R³ and R⁴ are C₁₄-C₂₀-alkenyl; wherein the heterocycloalkyl is substituted with R¹¹; R¹¹ is R¹⁵; and R¹⁵ is alkyl which is unsubstituted. Another embodiment of Formula (I) pertains to compounds wherein Y³ and Y⁴ are each a bond; R¹ and R² are each independently R⁵; one R⁵ is alkyl which is unsubstituted, and the other R⁵ is alkyl which is substituted with one OR⁶; R⁶ is R¹⁰; R¹⁰ is alkyl; and R³ and R⁴ are each C₁₄-C₂₀-alkenyl. Another embodiment of Formula (I) pertains to compounds wherein Y³ and Y⁴ are each a bond; R¹ and R² together with the nitrogen to which they are attached, are heterocycloalkyl; R³ and R⁴ are C₁₄-C₂₀-alkenyl; wherein the heterocycloalkyl is substituted with R¹¹; R¹¹ is R¹²; R¹² is phenyl which is unfused; wherein R¹² is substituted with one OR¹⁸; and R¹⁸ is alkyl. Another embodiment of Formula (I) pertains to compounds wherein Y³ and Y⁴ are each a bond; R¹ and R² are each independently R⁵; one R⁵is alkyl which is unsubstituted, and the other R⁵is alkyl which is substituted with one N(R⁶)₂; R⁶ is R¹⁰; R¹⁰ is alkyl; and R³ and R⁴ are each C₁₄-C₂₀-alkenyl. Another embodiment of Formula (I) pertains to compounds wherein Y³ and Y⁴ are each a bond; R¹ and R² are each independently R⁵; one R⁵ is alkyl which is unsubstituted, and the other R⁵ is alkyl which is substituted with one R⁶; R⁶ is R⁸; R⁸ is heteroaryl which is unfused; and R³ and R⁴ are each C₁₄-C₂₀-alkenyl. Another embodiment of Formula (I) pertains to compounds wherein Y³ and Y⁴ are each a bond; R¹ and R² are each independently R⁵; one R⁵ is alkyl which is unsubstituted, and the other R⁵ is alkyl which is substituted with one R⁶; R⁶ is R⁷; R⁷ is phenyl which is unfused; and R³ and R⁴ are each C₁₄-C₂₀-alkenyl. Another embodiment of Formula (I) pertains to compounds wherein Y³ and Y⁴ are each a bond; R¹ and R² are each independently R⁵; one R⁵ is alkyl which is unsubstituted, and the other R⁵is alkyl which is substituted with one R⁶; R⁶ is R⁷; R⁷ is phenyl which is unfused; and R³ and R⁴ are each C₁₄-C₂₀-alkenyl; wherein R⁷ is substituted with one F. Another embodiment of Formula (I) pertains to compounds wherein Y³ and Y⁴ are each a bond; R¹ and R² together with the nitrogen to which they are attached, are heterocycloalkyl; R³ and R⁴ are C₁₄-C₂₀-alkenyl; wherein the heterocycloalkyl is substituted with R¹¹; R¹¹ is R¹²; and R¹² is phenyl which is unfused; wherein R¹² is substituted with one F. Another embodiment of Formula (I) pertains to compounds wherein Y³ and Y⁴ are each a bond; R¹ and R² together with the nitrogen to which they are attached, are heterocycloalkyl; R³ and R⁴ are C₁₄-C₂₀-alkenyl; wherein the heterocycloalkyl is substituted with N(R¹¹)₂; R¹¹ is R¹⁵; and R¹⁵ is alkyl which is unsubstituted. Another embodiment of Formula (I) pertains to compounds wherein Y³ and Y⁴ are each a bond; R¹ and R² are each independently R⁵; each R⁵ is alkyl which is substituted with one OR⁶; R⁶ is R¹⁰; R¹⁰ is alkyl; and R³ and R⁴ are each C₁₄-C₂₀-alkenyl. Another embodiment of Formula (I) pertains to compounds wherein Y³ and Y⁴ are each a bond; R¹ and R² together with the nitrogen to which they are attached, are heterocycloalkyl; R³ and R⁴ are C₁₄-C₂₀-alkenyl; wherein the heterocycloalkyl is substituted with OR¹¹; R¹¹ is R¹⁵; and R¹⁵ is alkyl which is unsubstituted.Another embodiment of Formula (I) pertains to compounds wherein Y³ and Y⁴ are each C(O); R¹ and R² are each independently R⁵; R⁵is alkyl which is unsubstituted; and R³ and R⁴ are each C₁₄-C₂₀-alkenyl. Another embodiment of Formula (I) pertains to compounds wherein Y³ and Y⁴ are each a bond; R¹ and R² are each independently R⁵; R⁵is alkyl which is unsubstituted; and R³ and R⁴ together are CR²⁰R²¹, wherein R²⁰ and R²¹ are each C₁₄-C₂₀-alkenyl. Another embodiment of Formula (I) pertains to compounds wherein Y³ and Y⁴ are each C(O); R¹ and R² together with the nitrogen to which they are attached, are heterocycloalkyl; and R³ and R⁴ are C₁₄-C₂₀-alkenyl. Another embodiment of Formula (I) pertains to compounds wherein Y³ and Y⁴ are each a bond; R¹ and R² together with the nitrogen to which they are attached, are heterocycloalkyl; and R³ and R⁴ are C₁₄-C₂₀-alkyl. Another embodiment of Formula (I) pertains to compounds wherein Y³ and Y⁴ are each a bond; R¹ is H; R² is R⁵; R⁵is alkyl which is substituted with R⁶; R⁶ is R⁸; R⁸ is heteroaryl which is unfused; and R³ and R⁴ are C₁₄-C₂₀-alkenyl. Another embodiment of Formula (I) pertains to compounds wherein Y³ and Y⁴ are each a bond; R¹ and R² together with the nitrogen to which they are attached, are heterocycloalkyl; R³ and R⁴ are C₁₄-C₂₀-alkenyl; wherein the heterocycloalkyl is substituted with R¹¹; R¹¹ is R¹³; and R¹³ is heteroaryl which is unfused. Another embodiment of Formula (I) pertains to compounds wherein Y³ and Y⁴ are each a bond; R¹ is H; R² is R⁵; R⁵is alkyl which is substituted with R⁶; R⁶ is R⁹; R⁹ is heterocycloalkyl which is unfused; and R³ and R⁴ are C₁₄-C₂₀-alkenyl. Another embodiment of Formula (I) pertains to compounds wherein Y³ and Y⁴ are each a bond; R¹ is H; R² is R⁵; R⁵is alkyl which is substituted with N(R⁶)₂; R⁶ is R¹⁰; R¹⁰ is alkyl; and R³ and R⁴ are C₁₄-C₂₀-alkenyl. Another embodiment of Formula (I) pertains to compounds wherein Y³ and Y⁴ are each a bond; R¹ and R² together with the nitrogen to which they are attached, are heterocycloalkyl; R³ and R⁴ are C₁₄-C₂₀-alkenyl; wherein the heterocycloalkyl is substituted with two R¹¹; each R¹¹ is R¹⁵; and each R¹⁵ is alkyl which is unsubstituted.

### Formula II

One embodiment of this invention, therefore pertains to a cationic lipid or mixtures thereof, having Formula (II)

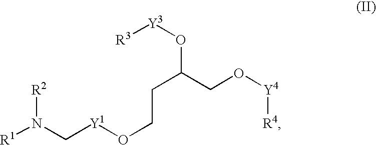

wherein Y³ and Y⁴ are each a bond. Another embodiment of Formula (II) pertains to compounds wherein Y³ and Y⁴ are each C(O). Another embodiment of Formula (II) pertains to compounds wherein Y³ is a bond and Y⁴ is C(O). Another embodiment of Formula (II) pertains to compounds wherein Y⁴ is a bond and Y³ is C(O).

One embodiment of Formula (II) pertains to compounds wherein R¹ and R² are each R⁵. Another embodiment of Formula (II) pertains to compounds wherein R¹ is H and R²is R⁵. Another embodiment of Formula (II) pertains to compounds wherein R¹ and R² together with the nitrogen to which they are attached, are heterocycloalkyl. Another embodiment of Formula (II) pertains to compounds wherein R¹ and R² together with the nitrogen to which they are attached, are heteroaryl. Another embodiment of Formula (II) pertains to compounds wherein R¹ and R² together with the nitrogen to which they are attached, are pyrrolidinyl, azetidinyl, or piperazinyl. Another embodiment of Formula (II) pertains to compounds wherein R¹ and R² together with the nitrogen to which they are attached, are pyrrolidinyl.

One embodiment of Formula (II) pertains to compounds wherein R³ and R⁴ are C₁₄-C₂₀-alkenyl. Another embodiment of Formula (II) pertains to compounds wherein R³ and R⁴ are C₁₄-C₂₀-alkyl. Another embodiment of Formula (II) pertains to compounds wherein R³ and R⁴ together are CR²⁰R²¹, wherein R²⁰ and R²¹ are each C₁₄-C₂₀-alkenyl. Another embodiment of Formula (II) pertains to compounds wherein R³is H; and R⁴ is C₁₄-C₂₀-alkenyl.

One embodiment of Formula (II) pertains to compounds wherein R⁵is alkyl. Another embodiment of Formula (II) pertains to compounds wherein R⁵is alkyl which is unsubstituted. Another embodiment of Formula (II) pertains to compounds wherein R⁵ is alkyl which is substituted. Another embodiment of Formula (II) pertains to compounds wherein R⁵ is alkyl which is substituted with R⁶, OR⁶, or N(R⁶)₂.

One embodiment of Formula (II) pertains to compounds wherein R⁶ is R⁷; and R⁷is phenyl which is unfused. Another embodiment of Formula (II) pertains to compounds wherein R⁶ is R⁸; and R⁸ is heteroaryl, which is unfused. Another embodiment of Formula (II) pertains to compounds wherein R⁶ is R⁹; and R⁹ is heterocycloalkyl, which is unfused. Another embodiment of Formula (II) pertains to compounds wherein R⁶ is R¹⁰; and R¹⁰ is alkyl, which is unsubstituted.

One embodiment of Formula (II) pertains to compounds wherein all foregoing cyclic moieties are unsubstituted. Another embodiment of Formula (II) pertains to compounds wherein one or more cyclic moieties are substituted. Another embodiment of Formula (II) pertains to compounds wherein one or more cyclic moieties are substituted with one or more R¹¹, OR¹¹, or N(R¹¹)₂, or F.

One embodiment pertains to compounds of Formula (II) wherein Y³ and Y⁴ are each a bond; Y¹ is C₁-C₆ alkylene; R¹ and R² are each independently R⁵; each R⁵ is alkyl which is unsubstituted; and R³ and R⁴ are C₁₄-C₂₀-alkenyl. Another embodiment of Formula (II) pertains to compounds wherein Y³ and Y⁴ are each a bond; Y¹ is C₁-C₆ alkylene; R¹ and R² together with the nitrogen to which they are attached, are heterocycloalkyl; R³ and R⁴ are C₁₄-C₂₀-alkenyl; wherein the heterocycloalkyl is substituted with one R¹¹; R¹¹ is R¹⁵; and R¹⁵ is alkyl which is unsubstituted. Another embodiment of Formula (II) pertains to compounds wherein Y³ and Y⁴ are each a bond; Y¹ is C₁-C₆ alkylene; R¹ and R² together with the nitrogen to which they are attached, are heterocycloalkyl; and R³ and R⁴ are C₁₄-C₂₀-alkenyl.

### Formula (III)

One embodiment of this invention, therefore pertains to a cationic lipid or mixtures thereof, having Formula (III)

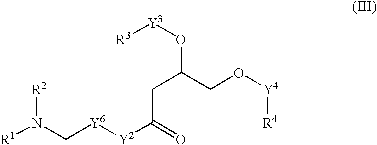

wherein Y² is NH. Another embodiment of Formula (III) pertains to compounds wherein Y² is O. Another embodiment of Formula (III) pertains to compounds wherein Y² is CH₂.

One embodiment of Formula (III) pertains to compounds wherein Y³ and Y⁴ are each a bond. Another embodiment of Formula (III) pertains to compounds wherein Y³ and Y⁴ are each C(O). Another embodiment of Formula (III) pertains to compounds wherein Y³ is a bond and Y⁴ is C(O). Another embodiment of Formula (III) pertains to compounds wherein Y⁴is a bond and Y³is C(O).

One embodiment of Formula (III) pertains to compounds wherein Y⁶ is a bond. Another embodiment of Formula (III) pertains to compounds wherein Y⁶ is C₁-C₆ alkylene.

One embodiment of Formula (III) pertains to compounds wherein R¹ and R² are each R⁵ Another embodiment of Formula (III) pertains to compounds wherein R¹ is H and R² is R⁵ Another embodiment of Formula (III) pertains to compounds wherein R¹ and R² together with the nitrogen to which they are attached, are heterocycloalkyl. Another embodiment of Formula (III) pertains to compounds wherein R¹ and R² together with the nitrogen to which they are attached, are heteroaryl.

One embodiment of Formula (III) pertains to compounds wherein R³ and R⁴ are C₁₄-C₂₀-alkenyl. Another embodiment of Formula (III) pertains to compounds wherein R³ and R⁴ are C₁₄-C₂₀-alkyl. Another embodiment of Formula (III) pertains to compounds wherein R³ and R⁴ together are CR²⁰R²¹, wherein R²⁰ and R²¹ are each C₁₄-C₂₀-alkenyl. Another embodiment of Formula (III) pertains to compounds wherein R³is H; and R⁴ is C₁₄-C₂₀-alkenyl.

One embodiment of Formula (III) pertains to compounds wherein R⁵is alkyl. Another embodiment of Formula (III) pertains to compounds wherein R⁵is alkyl which is unsubstituted. Another embodiment of Formula (III) pertains to compounds wherein R⁵ is alkyl which is substituted. Another embodiment of Formula (III) pertains to compounds wherein R⁵ is alkyl which is substituted with R⁶, OR⁶, or N(R⁶)₂.

One embodiment of Formula (III) pertains to compounds wherein R⁶ is R⁷; and R⁷ is phenyl which is unfused. Another embodiment of Formula (III) pertains to compounds wherein R⁶ is R⁸; and R⁸ is heteroaryl, which is unfused. Another embodiment of Formula (III) pertains to compounds wherein R⁶ is R⁹; and R⁹ is heterocycloalkyl, which is unfused. Another embodiment of Formula (III) pertains to compounds wherein R⁶ is R¹⁰; and R¹⁰ is alkyl, which is unsubstituted.

One embodiment of Formula (III) pertains to compounds wherein all foregoing cyclic moieties are unsubstituted. Another embodiment of Formula (III) pertains to compounds wherein one or more cyclic moieties are substituted. Another embodiment of Formula (III) pertains to compounds wherein one or more cyclic moieties are substituted with one or more R¹¹, OR¹¹, or N(R¹¹)₂, or F.

### Formula (IV)

Another embodiment of this invention, therefore pertains to a cationic lipid or mixtures thereof, having Formula (IV)

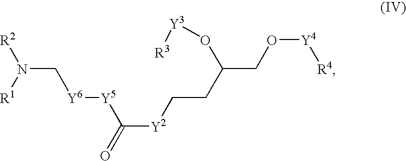

wherein Y² is NH. Another embodiment of Formula (IV) pertains to compounds wherein Y² is O. Another embodiment of Formula (IV) pertains to compounds wherein Y²is CH₂.

One embodiment of Formula (IV) pertains to compounds wherein Y³ and Y⁴ are each a bond. Another embodiment of Formula (IV) pertains to compounds wherein Y³ and Y⁴ are each C(O). Another embodiment of Formula (IV) pertains to compounds wherein Y³ is a bond and Y⁴ is C(O). Another embodiment of Formula (IV) pertains to compounds wherein Y⁴is a bond and Y³is C(O).

One embodiment of Formula (IV) pertains to compounds wherein Y⁵ is CH₂. Another embodiment of Formula (IV) pertains to compounds wherein Y⁵ is NH. Another embodiment of Formula (IV) pertains to compounds wherein Y⁵ is O.

One embodiment of Formula (IV) pertains to compounds wherein Y⁶ is a bond. Another embodiment of Formula (IV) pertains to compounds wherein Y⁶ is C₁-C₆ alkylene.

One embodiment of Formula (IV) pertains to compounds wherein R¹ and R² are each R⁵. Another embodiment of Formula (IV) pertains to compounds wherein R¹ is H and R²is R⁵. Another embodiment of Formula (IV) pertains to compounds wherein R¹ and R² together with the nitrogen to which they are attached, are heterocycloalkyl. Another embodiment of Formula (IV) pertains to compounds wherein R¹ and R² together with the nitrogen to which they are attached, are heteroaryl. Another embodiment of Formula (IV) pertains to compounds wherein R¹ and R² together with the nitrogen to which they are attached, is pyrrolidinyl.

One embodiment of Formula (IV) pertains to compounds wherein R³ and R⁴ are C₁₄-C₂₀-alkenyl. Another embodiment of Formula (IV) pertains to compounds wherein R³ and R⁴ are C₁₄-C₂₀-alkyl. Another embodiment of Formula (IV) pertains to compounds wherein R³ and R⁴ together are CR²⁰R²¹, wherein R²⁰ and R²¹ are each C₁₄-C₂₀-alkenyl. Another embodiment of Formula (IV) pertains to compounds wherein R³is H; and R⁴ is C₁₄-C₂₀-alkenyl.

One embodiment of Formula (IV) pertains to compounds wherein R⁵is alkyl. Another embodiment of Formula (IV) pertains to compounds wherein R⁵ is alkyl which is unsubstituted. Another embodiment of Formula (IV) pertains to compounds wherein R⁵ is alkyl which is substituted. Another embodiment of Formula (IV) pertains to compounds wherein R⁵ is alkyl which is substituted with R⁶, OR⁶, or N(R⁶)₂.

One embodiment of Formula (IV) pertains to compounds wherein R⁶ is R⁷; and R⁷ is phenyl which is unfused. Another embodiment of Formula (IV) pertains to compounds wherein R⁶ is R⁸; and R⁸ is heteroaryl, which is unfused. Another embodiment of Formula (IV) pertains to compounds wherein R⁶is R⁹; and R⁹ is heterocycloalkyl, which is unfused. Another embodiment of Formula (IV) pertains to compounds wherein R⁶ is R¹⁰; and R¹⁰ is alkyl, which is unsubstituted.

One embodiment of Formula (IV) pertains to compounds wherein all foregoing cyclic moieties are unsubstituted. Another embodiment of Formula (IV) pertains to compounds wherein one or more cyclic moieties are substituted. Another embodiment of Formula (IV) pertains to compounds wherein one or more cyclic moieties are substituted with one or more R¹¹, OR¹¹, or N(R¹¹)₂, or F.

One embodiment pertains to compounds of Formula (IV) wherein Y³ and Y⁴ are each a bond; Y² is O; Y⁵ is NH; Y⁶ is C₁-C₆ alkylene; R¹ and R² together with the nitrogen to which they are attached are heterocycloalkyl; and R³ and R⁴ are C₁₄-C₂₀-alkenylAnother embodiment of Formula (IV) pertains to compounds wherein Y³ and Y⁴ are each a bond; Y² is NH; Y⁵ is CH₂; Y⁶ is a bond; R¹ and R² are each independently R⁵; each R⁵ is alkyl which is unsubstituted; and R³ and R⁴ are C₁₄-C₂₀-alkenyl.

Still another embodiment pertains to compounds of this invention which include, but are not limited to 1-{3,4-bis[(9Z,12Z)-octadeca-9,12-dienyloxy]butyl}piperidine, 4-{3,4-bis[(9Z,12Z)-octadeca-9,12-dienyloxy]butyl}morpholine, N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, 1-{3,4-bis[(9Z,12Z)-octadeca-9,12-dienyloxy]butyl}-4-phenylpiperazine, 1-{3,4-bis[(9Z,12Z)-octadeca-9,12-dienyloxy]butyl}-4-methylpiperazine, N-(2-methoxyethyl)-N-methyl-3,4-bis[(9Z,12Z)-octadeca-9,12-dienyloxy]butan-1-amine, 1-{3,4-bis [(9Z,12Z)-octadeca-9,12-dienyloxy]butyl}-4-(2-methoxyphenyl)piperazine, N-{3,4-bis[(9Z,12Z)-octadeca-9,12-dienyloxy]butyl}-N,N′,N′-trimethylethane-1,2-diamine, N-{3,4-bis[(9Z,12Z)-octadeca-9,12-dienyloxy]butyl}-N-methyl-N-(2-pyridin-2-ylethyl)amine, N-benzyl-N-{3,4-bis[(9Z,12Z)-octadeca-9,12-dienyloxy]butyl}-N-methylamine, N-{3,4-bis[(9Z,12Z)-octadeca-9,12-dienyloxy]butyl}-N-(4-fluorobenzyl)-N-methylamine, 1-{3,4-bis[(9Z,12Z)-octadeca-9,12-dienyloxy]butyl}-4-(2-fluorophenyl)piperazine, N-benzyl-N-{3,4-bis[(9Z,12Z)-octadeca-9,12-dienyloxy]butyl}-N-ethylamine, N-{3,4-bis[(9Z,12Z)-octadeca-9,12-dienyloxy]butyl}-N-ethyl-N′,N′-dimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, N,N-bis(2-methoxyethyl)-3,4-bis[(9Z,12Z)-octadeca-9,12-dienyloxy]butan-1-amine, 1-{3,4-bis[(9Z,12Z)-octadeca-9,12-dienyloxy]butyl}-4-methoxypiperidine, 1-{(3R)-3,4-bis[(9Z,12Z)-octadeca-9,12-dienyloxy]butyl}pyrrolidine, 1-{(3S)-3,4-bis[(9Z,12Z)-9,12-dienyloxy]butyl}pyrrolidine, N-{(3R)-3,4-bis[(9Z,12Z)-octadeca-9,12-dienyloxy]butyl}-N,N-diethylamine, N-{(3S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 2-(2-{3,4-bis[(9Z,12Z)-octadeca-9,12-dienyloxy]butoxy}ethyl)-1-methylpyrrolidine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis[(9Z,12Z)-octadeca-9,12-dienyloxy]butoxy}ethyl)-N,N-dimethylamine, 4-(diethylamino)-2-[(9Z,12Z)-octadeca-9,12-dienoyloxy]butyl (9Z,12Z)-octadeca-9,12-dienoate, 1-(2-{3,4-bis[(9Z,12Z)-octadeca-9,12-dienyloxy]butoxy}ethyl)pyrrolidine, N,N-diethyl-N-(2-{2-[(8Z,11Z)-heptadeca-8,11-dienyl]-2-[(9Z,12Z)-octadeca-9,12-dienyl]-1,3-dioxolan-4-yl}ethyl)amine, 1-{[(9Z)-octadec-9-enoyloxy]methyl}-3-pyrrolidin-1-ylpropyl (9Z)-octadec-9-enoate, 1-{3,4-bis[(9Z)-octadec-9-enyloxy]butyl}pyrrolidine, 1-{[(5Z,8Z,11Z,14Z)-icosa-5,8,11,14-tetraenoyloxy]methyl}-3-pyrrolidin-1-ylpropyl (5Z,8Z,11Z,14Z)-icosa-5,8,11,14-tetraenoate, (3S)-3,4-bis[(9Z,12Z)-octadeca-9,12-dienyloxy]butyl 3-pyrrolidin-1-ylpropylcarbamate, 1-[3,4-bis(octadecyloxy)butyl]pyrrolidine, 1-[3,4-bis(hexadecyloxy)butyl]pyrrolidine, 1-{3,4-bis[(9E)-hexadec-9-enyloxy]butyl}pyrrolidine, 1-{3,4-bis[(9E)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N¹-{(3S)-3,4-bis[(9Z,12Z)-octadeca-9,12-dienyloxy]butyl}-N³,N³-diethyl-beta-alaninamide, N-{3,4-bis[(9Z,12Z)-octadeca-9,12-dienyloxy]butyl}-N-[3-(1H-imidazol-1-yl)propyl]amine, N-{3,4-bis[(9Z,12Z)-octadeca-9,12-dienyloxy]butyl}-N,N′,N′-trimethylpropane-1,3-diamine, 1-(1-{3,4-bis[(9Z,12Z)-octadeca-9,12-dienyloxy]butyl}pyrrolidin-3-yl)-1H-imidazole, N-{3,4-bis[(9Z,12Z)-octadeca-9,12-dienyloxy]butyl}-N-(3-pyrrolidin-1-ylpropyl)amine, N-{3,4-bis[(9Z,12Z)-octadeca-9,12-dienyloxy]butyl}-N′,N′-dimethylpropane-1,3-diamine, 1-{3,4-bis[(9Z,12Z)-octadeca-9,12-dienyloxy]butyl}azetidine, 1-{3,4-bis[(9Z,12Z)-octadeca-9,12-dienyloxy]butyl}-2-methylpyrrolidine, and 1-{3,4-bis[(9Z,12Z)-octadeca-9,12-dienyloxy]butyl}-2,5-dimethylpyrrolidine.

Still another embodiment pertains to compounds of this invention wherein one or more cationic lipids are chosen from 1-{(3S)-3,4-bis[(9Z,12Z)-octadeca-9,12-dienyloxy]butyl}pyrrolidine, 1-{(3R)-3,4-bis[(9Z,12Z)-octadeca-9,12-dienyloxy]butyl}pyrrolidine, 1-{3,4-bis[(9Z,12Z)-octadeca-9,12-dienyloxy]butyl}-N,N-dimethylpyrrolidin-3-amine, and 1-{3,4-bis[(9Z,12Z)-octadeca-9,12-dienyloxy]butyl}pyrrolidine.

Still another embodiment pertains to compounds of this invention wherein one or more cationic lipids are chosen from N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, N-(2-methoxyethyl)-N-methyl-3,4-bis[(9Z,12Z)-octadeca-9,12-dienyloxy]butan-1-amine, N-{3,4-bis[(9Z,12Z)-octadeca-9,12-dienyloxy]butyl}-N,N′,N′-trimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, 1-{(3R)-3,4-bis[(9Z,12Z)-octadeca-9,12-dienyloxy]butyl}pyrrolidine, 1-{(3S)-3,4-bis[(9Z,12Z)-octadeca-9,12-dienyloxy]butyl}pyrrolidine, N-{(3R)-3,4-bis[(9Z,12Z)-octadeca-9,12-dienyloxy]butyl}-N,N-diethylamine, N-{(3S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis[(9Z,12Z)-octadeca-9,12-dienyloxy]butoxy}ethyl)-N,N-dimethylamine, 1-(2-{3,4-bis[(9Z,12Z)-octadeca-9,12-dienyloxy]butoxy}ethyl)pyrrolidine, N,N-diethyl-N-(2-{2-[(8Z,1 Z)-heptadeca-8,11-dienyl]-2-[(9Z,12Z)-octadeca-9,12-dienyl]-1,3-dioxolan-4-yl}ethyl)amine, 1-{3,4-bis[(9Z)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N-{3,4-bis[(9Z,12Z)-octadeca-9,12-dienyloxy]butyl}-N,N′,N′-trimethylpropane-1,3-diamine, and 1-{3,4-bis[(9Z,12Z)-octadeca-9,12-dienyloxy]butyl}azetidine.

### Particles, Cationic-Based Lipid Encapsulation Systems, and Lipid-Based Particles

A further embodiment pertains to particles comprising one or more cationic lipid(s) having Formula I, II, III, or IV.

A further embodiment pertains to particles comprising one or more cationic lipid(s) having Formula I, II, III, or IV and one or more therapeutic agents. Preferably said therapeutic agent is a nucleic acid encoded with a product of interest, including but not limited to, RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), antigens, fragments thereof, proteins, peptides, and small-molecules.

A further embodiment pertains to nanoparticles comprising one or more cationic lipid(s) having Formula I, II, III, or IV.

A further embodiment pertains to nanoparticles comprising one or more cationic lipid(s) having Formula I, II, III, or IV and one or more therapeutic agents. Preferably said therapeutic agent is a nucleic acid encoded with a product of interest, including but not limited to, RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), antigens, fragments thereof, proteins, peptides, and small-molecules.

A still further embodiment pertains to Cationic-Based Lipid Encapsulation Systems (CaBLES) comprising non-cationic lipid(s), polyethylene glycol (PEG)-lipid conjugate(s) and cationic lipid(s) having Formula I, II, III, or IV.

A still further embodiment pertains to Cationic-Based Lipid Encapsulation Systems (CaBLES) comprising one or more cationic lipids having Formula (I)

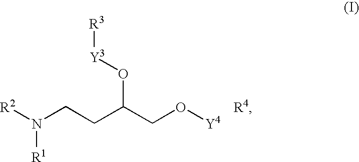

### Formula (II)

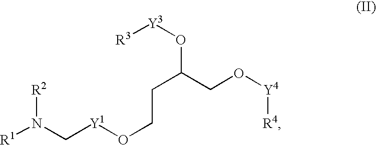

### Formula (III)

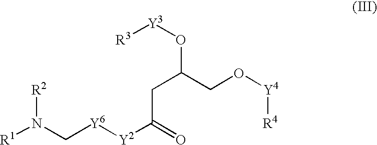

or

### Formula (IV)

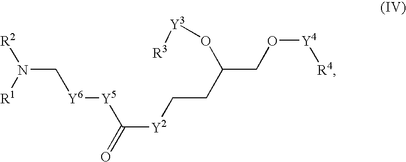

wherein Y¹ is C₁-C₆ alkylene;

Y² is CH₂, NH or O;

Y³ is a bond or C(O);

Y⁴ is a bond or C(O);

Y⁵ is CH₂, NH or O;

Y⁶ is a bond or C₁-C₆ alkylene;

R¹ and R² are independently H, cycloalkyl, cycloalkenyl or R⁵; or

R¹ and R², together with the nitrogen to which they are attached, are heterocycloalkyl or heteroaryl;

one of R³ and R⁴ is H, and the other is C₁₄-C₂₀-alkenyl, or C₁₄-C₂₀-alkyl; or R³ and R⁴ are independently selected C₁₄-C₂₀-alkenyl, or C₁₄-C₂₀-alkyl; or

R³ and R⁴ together are CR²⁰R²¹, wherein R²⁰ is H and R²¹ is C₁₄-C₂₀-alkenyl, C₁₄-C₂₀-alkyl, or CH₂O—C₁₄-C₂₀-alkenyl; or R²⁰ and R²¹ are independently selected C₁₄-C₂₀-alkenyl, C₁₄-C₂₀-alkyl, or CH₂O—C₁₄-C₂₀-alkenyl;

R⁶ is alkyl, which is unsubstituted or substituted with one or more R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, C(N)NH₂, C(N)NHR⁶, C(N)N(R⁶)₂, CNOH, CNOCH₃, OH, (O), CN, N₃, NO₂, CF₃, CF₂CF₃, OCF₃, OCF₂CF₃, F, Cl, Br or I;

R⁶ is R⁷, R⁸, R⁹, or R¹⁰;

R⁷ is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁸ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁹ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁰ is alkyl, alkenyl or alkynyl;

wherein each foregoing cyclic moiety is independently unsubstituted or substituted with one or more R¹¹, OR¹¹, SR¹¹, S(O)R¹¹, SO₂R¹¹, C(O)R¹¹, CO(O)R¹¹, OC(O)R¹¹, OC(O)OR¹¹, NH₂, NHR¹¹, N(R¹¹)₂, NHC(O)R¹¹, NR¹¹C(O)R¹¹, NHS(O)₂R¹¹, NR¹¹S(O)₂R¹¹, NHC(O)OR¹¹, NR¹¹C(O)OR¹¹, NHC(O)NH₂, NHC(O)NHR¹¹, NHC(O)N(R¹¹)₂, NR¹¹C(O)NHR¹¹, NR¹¹C(O)N(R¹¹)₂, C(O)NH₂, C(O)NHR¹¹, C(O)N(R¹¹)₂, C(O)NHOH, C(O)NHOR¹¹, C(O)NHSO₂R¹¹, C(O)NR¹¹SO₂R¹¹, SO₂NH₂, SO₂NHR¹¹, SO₂N(R¹¹)₂, C(O)H, C(O)OH, C(N)NH₂, C(N)NHR¹¹, C(N)N(R¹¹)₂, CNOH, CNOCH₃, OH, (O), CN, N₃, NO₂, CF₃, CF₂CF₃, OCF₃, OCF₂CF₃, F, Cl, Br or I;

R¹¹ is R¹², R¹³, R¹⁴, or R¹⁵;

R¹² is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹³ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁴ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁵ is alkyl, alkenyl or alkynyl, each of which is unsubstituted or substituted with one or two of independently selected R¹⁶, OR^{16E}, SR¹⁶, S(O)₂R¹⁶, C(O)OH, NH₂, NHR¹⁶ N(R¹⁶)₂, C(O)R¹⁶, C(O)NH₂, C(O)NHR¹⁶, C(O)N(R¹⁶)₂, NHC(O)R¹⁶, NR¹⁶C(O)R¹⁶, NHC(O)OR¹⁶, NR¹⁶C(O)OR¹⁶, OH, F, Cl, Br or I;

R¹⁶ is alkyl, alkenyl, alkynyl, or R¹⁷;

R¹⁷ is phenyl, heteroaryl, cycloalkyl, cycloalkenyl, heterocycloalkyl or heterocycloalkenyl;

wherein R¹², R¹³, R¹⁴, and R¹⁷ are independently unsubstituted or substituted with one or more R¹⁸, OR¹⁸, SR¹⁸, S(O)R¹⁸, SO₂R¹⁸, C(O)R¹⁸, CO(O)R¹⁸, OC(O)R¹⁸, OC(O)OR¹⁸, NH₂, NHR¹⁸, N(R¹⁸)₂, NHC(O)R¹⁸, NR¹⁸C(O)R¹⁸, NHS(O)₂R¹⁸, NR¹⁸S(O)₂R¹⁸, NHC(O)OR¹⁸, NR¹⁸C(O)OR¹⁸, NHC(O)NH₂, NHC(O)NHR¹⁸, NHC(O)N(R¹⁸)₂, NR¹⁸C(O)NHR¹⁸, NR¹⁸C(O)N(R¹⁸)₂, C(O)NH₂, C(O)NHR¹⁸, C(O)N(R¹⁸)₂, C(O)NHOH, C(O)NHOR¹⁸, C(O)NHSO₂R¹⁸, C(O)NR¹⁸SO₂R¹⁸, SO₂NH₂, SO₂NHR¹⁸, SO₂N(R¹⁸)₂, C(O)H, C(O)OH, C(N)NH₂, C(N)NHR¹⁸, C(N)N(R¹⁸)₂, CNOH, CNOCH₃, OH, (O), CN, N₃, NO₂, CF₃, CF₂CF₃, OCF₃, OCF₂CF₃, F, Cl, Br or I;

R¹⁸ is alkyl, alkenyl, alkynyl, phenyl, heteroaryl, cycloalkyl, cycloalkenyl, heterocycloalkyl or heterocycloalkenyl; and

one or more non-cationic lipids, and one or more polyethylene glycol-lipid conjugates.

In still a further embodiment, Lipid-Based Particles of the present invention are defined as CaBLES which further comprise one or more therapeutic agent(s). Therapeutic agents that can be delivered with CaBLES include RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), chimeric nucleic acids, an antigen, fragments thereof, a protein, a peptide, small-molecules, or mixtures thereof. This invention describes delivery of RNA's such as small inhibitory RNA or microRNA. The nucleic acid can have varying lengths (10-200 bps) and structures (hairpins, single/double strands, bulges, nicks/gaps, mismatches) and processed in the cell to provide active gene silencing. In certain embodiments of this invention, a double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

In certain embodiments, the PEG lipid conjugate of the Lipid-Based Particle can have a ligand attached, such as a targeting ligand or a chelating moiety. Suitable targeting ligands include, but are not limited to, a compound or device with a reactive functional group and include lipids, amphipathic lipids, carrier compounds, bioaffinity compounds, biomaterials, biopolymers, biomedical devices, analytically detectable compounds, therapeutically active compounds, enzymes, peptides, proteins, antibodies, immune stimulators, radiolabels, fluorogens, biotin, drugs, haptens, DNA, RNA, polysaccharides, liposomes, virosomes, micelles, immunoglobulins, functional groups, other targeting moieties, or toxins.

In another embodiment, a targeting ligand (moiety) is conjugated to the periphery of the PEG-lipid in a Lipid-Based Particle formulation. Preferably, the targeting moiety is a ligand of a receptor present on a target cell and the receptor is preferentially expressed by the target cell versus a non-target cell. In one aspect, the targeting moiety is an antibody or fragments thereof. In one aspect, the targeting moiety is a small protein, or peptide. In another aspect, the targeting moiety is a small-molecule.

In still a further embodiment, these Lipid-Based Particles are nanoparticles and have mean diameter sizes of about 50-300 nm, of which 50-250 nm is preferred and 50-200 nm is most preferred.

A further embodiment pertains to CaBLES or Lipid-Base Particles wherein the PEG lipid conjugate is about 0.1-20 weight/weight % of total lipid in particle, the non-cationic lipid is about 1-30 weight/weight % of total lipid in particle, the cholesterol is about 5-45 weight/weight % of total lipid in particle, and the cationic lipid is about 5-60 weight/weight % of total lipid in particle.

A further embodiment pertains to CaBLES or Lipid-Base Particles wherein the PEG lipid conjugate is about 0.1-20 weight/weight % of total lipid in particle, the DSPC is about 1-30 weight/weight % of total lipid in particle, the cholesterol is about 5-45 weight/weight % of total lipid in particle, and the cationic lipid is about 5-60 weight/weight % of total lipid in particle.

A further embodiment pertains to a pharmaceutical composition comprising a Lipid-Based Particle and a pharmaceutically acceptable carrier.

A further embodiment pertains to a pharmaceutical composition, wherein the Lipid-Based Particle comprises, cholesterol, DSPC, 1-{(3S)-3,4-bis[(9Z,12Z)-octadeca-9,12-dienyloxy]butyl}pyrrolidine, one or more PEG-lipid conjugates, and one or more nucleic acids.

A further embodiment pertains to a pharmaceutical composition, wherein the (PEG)-lipid conjugates are about 0.1-20 weight/weight % of total lipid in particle, the DSPC is about 1-30 weight/weight % of total lipid in particle, the cholesterol is about 5-45 weight/weight % of total lipid in particle, and 1-{(3S)-3,4-bis[(9Z,12Z)-octadeca-9,12-dienyloxy]butyl}pyrrolidine is about 5-60 weight/weight % of total lipid in particle.

A further embodiment pertains to a Lipid-Based Particle, wherein the non-cationic lipids are cholesterol and DSPC, the cationic lipid is 1-{(3S)-3,4-bis[(9Z,12Z)-octadeca-9,12-dienyloxy]butyl}pyrrolidine, the PEG-lipid conjugate is N-(2,3-dimyristyloxypropyl)carbamate polyethyleneglycol-2000 methyl ether, and the therapeutic agent is siRNA.

A further embodiment pertains to a Lipid-Based Particle, wherein the N-(2,3-dimyristyloxypropyl)carbamate polyethyleneglycol-2000 methyl ether is about 0.1-20 weight/weight % of total lipid in particle, the DSPC is about 1-30 weight/weight % of total lipid in particle, the cholesterol is about 5-45 weight/weight % of total lipid in particle, and the 1-{(3S)-3,4-bis[(9Z,12Z)-octadeca-9,12-dienyloxy]butyl}pyrrolidine is about 5-60 weight/weight % of total lipid in particle.

A further embodiment pertains to a pharmaceutical composition, wherein the Lipid-Based Particle comprises, cholesterol, DSPC, 1-{(3R)-3,4-bis[(9Z,12Z)-octadeca-9,12-dienyloxy]butyl}pyrrolidine, one or more PEG-lipid conjugates, and one or more nucleic acids.

A further embodiment pertains to a pharmaceutical composition, wherein the (PEG)-lipid conjugates are about 0.1-20 weight/weight % of total lipid in particle, the DSPC is about 1-30 weight/weight % of total lipid in particle, the cholesterol is about 5-45 weight/weight % of total lipid in particle, and 1-{(3R)-3,4-bis[(9Z,12Z)-octadeca-9,12-dienyloxy]butyl}pyrrolidine is about 5-60 weight/weight % of total lipid in particle.

A further embodiment pertains to a Lipid-Based Particle, wherein the non-cationic lipids are cholesterol and DSPC, the cationic lipid is 1-{(3R)-3,4-bis[(9Z,12Z)-octadeca-9,12-dienyloxy]butyl}pyrrolidine, the PEG-lipid conjugate is N-(2,3-dimyristyloxypropyl)carbamate polyethyleneglycol-2000 methyl ether, and the therapeutic agent is siRNA.

A further embodiment pertains to a Lipid-Based Particle, wherein the N-(2,3-dimyristyloxypropyl)carbamate polyethyleneglycol-2000 methyl ether is about 0.1-20 weight/weight % of total lipid in particle, the DSPC is about 1-30 weight/weight % of total lipid in particle, the cholesterol is about 5-45 weight/weight % of total lipid in particle, and the 1-{(3R)-3,4-bis[(9Z,12Z)-octadeca-9,12-dienyloxy]butyl}pyrrolidine is about 5-60 weight/weight % of total lipid in particle.

A further embodiment pertains to a pharmaceutical composition, wherein the Lipid-Based Particle comprises, cholesterol, DSPC, 1-{3,4-bis[(9Z,12Z)-octadeca-9,12-dienyloxy]butyl}-N,N-dimethylpyrrolidin-3-amine, one or more PEG-lipid conjugates, and one or more nucleic acids.

A further embodiment pertains to a pharmaceutical composition, wherein the (PEG)-lipid conjugates are about 0.1-20 weight/weight % of total lipid in particle, the DSPC is about 1-30 weight/weight % of total lipid in particle, the cholesterol is about 5-45 weight/weight % of total lipid in particle, and 1-{3,4-bis[(9Z,12Z)-octadeca-9,12-dienyloxy]butyl}-N,N-dimethylpyrrolidin-3-amine is about 5-60 weight/weight % of total lipid in particle.

A further embodiment pertains to a Lipid-Based Particle, wherein the non-cationic lipids are cholesterol and DSPC, the cationic lipid is 1-{3,4-bis[(9Z,12Z)-octadeca-9,12-dienyloxy]butyl}-N,N-dimethylpyrrolidin-3-amine, the PEG-lipid conjugate is N-(2,3-dimyristyloxypropyl)carbamate polyethyleneglycol-2000 methyl ether, and the therapeutic agent is siRNA.

A further embodiment pertains to a Lipid-Based Particle, wherein the N-(2,3-dimyristyloxypropyl)carbamate polyethyleneglycol-2000 methyl ether is about 0.1-20 weight/weight % of total lipid in particle, the DSPC is about 1-30 weight/weight % of total lipid in particle, the cholesterol is about 5-45 weight/weight % of total lipid in particle, and the 1-{3,4-bis[(9Z,12Z)-octadeca-9,12-dienyloxy]butyl}-N,N-dimethylpyrrolidin-3-amine is about 5-60 weight/weight % of total lipid in particle.

A further embodiment pertains to a pharmaceutical composition, wherein the Lipid-Based Particle comprises, cholesterol, DSPC, 1-{3,4-bis[(9Z,12Z)-octadeca-9,12-dienyloxy]butyl}pyrrolidine, one or more PEG-lipid conjugates, and one or more nucleic acids.

A further embodiment pertains to a pharmaceutical composition, wherein the (PEG)-lipid conjugates are about 0.1-20 weight/weight % of total lipid in particle, the DSPC is about 1-30 weight/weight % of total lipid in particle, the cholesterol is about 5-45 weight/weight % of total lipid in particle, and 1-{3,4-bis[(9Z,12Z)-octadeca-9,12-dienyloxy]butyl}pyrrolidine is about 5-60 weight/weight % of total lipid in particle.

A further embodiment pertains to a Lipid-Based Particle, wherein the non-cationic lipids are cholesterol and DSPC, the cationic lipid is 1-{3,4-bis[(9Z,12Z)-octadeca-9,12-dienyloxy]butyl}pyrrolidine, the PEG-lipid conjugate is 2-(octadecyloxy)-1-((octadecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate, and the therapeutic agent is siRNA.

A further embodiment pertains to a Lipid-Based Particle, wherein the 2-(octadecyloxy)-1-((octadecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate is about 0.1-20 weight/weight % of total lipid in particle, the DSPC is about 1-30 weight/weight % of total lipid in particle, the cholesterol is about 5-45 weight/weight % of total lipid in particle, and the 1-{3,4-bis[(9Z,12Z)-octadeca-9,12-dienyloxy]butyl}pyrrolidine is about 5-60 weight/weight % of total lipid in particle.

A further embodiment pertains to a Lipid-Based Particle, wherein the non-cationic lipids are cholesterol and DSPC, the cationic lipid is 1-{3,4-bis[(9Z,12Z)-octadeca-9,12-dienyloxy]butyl}pyrrolidine, the PEG-lipid conjugate is N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, and the therapeutic agent is siRNA.

A further embodiment pertains to a Lipid-Based Particle, wherein the N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide is about 0.1-20 weight/weight % of total lipid in particle, the DSPC is about 1-30 weight/weight % of total lipid in particle, the cholesterol is about 5-45 weight/weight % of total lipid in particle, and the 1-{3,4-bis[(9Z,12Z)-octadeca-9,12-dienyloxy]butyl}pyrrolidine is about 5-60 weight/weight % of total lipid in particle.

A further embodiment pertains to a Lipid-Based Particle, wherein the non-cationic lipids are cholesterol and DSPC, the cationic lipid is 1-{3,4-bis[(9Z,12Z)-octadeca-9,12-dienyloxy]butyl}pyrrolidine, the PEG-lipid conjugate is N-[3,4-bis(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, and the therapeutic agent is siRNA.

A further embodiment pertains to a Lipid-Based Particle, wherein the N-[3,4-bis(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide is about 0.1-20 weight/weight % of total lipid in particle, the DSPC is about 1-30 weight/weight % of total lipid in particle, the cholesterol is about 5-45 weight/weight % of total lipid in particle, and the 1-{3,4-bis[(9Z,12Z)-octadeca-9,12-dienyloxy]butyl}pyrrolidine is about 5-60 weight/weight % of total lipid in particle.

A further embodiment pertains to a Lipid-Based Particle, wherein the non-cationic lipids are cholesterol and DSPC, the cationic lipid is 1-{3,4-bis[(9Z,12Z)-octadeca-9,12-dienyloxy]butyl}pyrrolidine, the PEG-lipid conjugate is 1,2-dipalmitoyl-sn-glycerol-methoxypolyethyleneglycol-2000, and the therapeutic agent is siRNA.

A further embodiment pertains to a Lipid-Based Particle, wherein the 1,2-dipalmitoyl-sn-glycerol-methoxypolyethyleneglycol-2000 is about 0.1-20 weight/weight % of total lipid in particle, the DSPC is about 1-30 weight/weight % of total lipid in particle, the cholesterol is about 5-45 weight/weight % of total lipid in particle, and the 1-{3,4-bis[(9Z,12Z)-octadeca-9,12-dienyloxy]butyl}pyrrolidine is about 5-60 weight/weight % of total lipid in particle.

A further embodiment pertains to a Lipid-Based Particle, wherein the non-cationic lipids are cholesterol and DSPC, the cationic lipid is 1-{3,4-bis[(9Z,12Z)-octadeca-9,12-dienyloxy]butyl}pyrrolidine, the PEG-lipid conjugate is N-(carbonyl-methoxypolyethyleneglycol-2000)-1,2-dipalmitoyl-sn-glycero-3-phosphoethanolamine, and the therapeutic agent is siRNA.

A further embodiment pertains to a Lipid-Based Particle, wherein the N-(carbonyl-methoxypolyethyleneglycol-2000)-1,2-dipalmitoyl-sn-glycero-3-phosphoethanolamine is about 0.1-20 weight/weight % of total lipid in particle, the DSPC is about 1-30 weight/weight % of total lipid in particle, the cholesterol is about 5-45 weight/weight % of total lipid in particle, and the 1-{3,4-bis[(9Z,12Z)-octadeca-9,12-dienyloxy]butyl}pyrrolidine is about 5-60 weight/weight % of total lipid in particle.

A further embodiment pertains to Lipid-Based Particles, wherein the ratio of one or more (PEG)-lipid conjugates, one or more non-cationic lipids, and one or more cationic lipids of Formula (I), to one or more therapeutic agents is between about 50:1 to about 5:1.

A further embodiment pertains to Lipid-Based Particles, wherein the ratio of one or more (PEG)-lipid conjugates, one or more non-cationic lipids, and one or more cationic lipids of Formula (I), to one or more therapeutic agents is between about 30:1 to about 10:1.

In still a further embodiment, functional CaBLES comprising one or more (PEG)-lipid conjugates, one or more non-cationic lipids, and one or more cationic lipids of Formula I, II, III, or IV effectively encapsulate nucleic acids, such as siRNA, with efficiencies from about 50-100%.

In still a further embodiment, functional CaBLES comprising one or more (PEG)-lipid conjugates, one or more non-cationic lipids, and one or more cationic lipids of Formula I, II, III, or IV effectively encapsulate nucleic acids, such as siRNA, with efficiencies from about 80-100%.

In still a further embodiment, functional CaBLES comprising one or more (PEG)-lipid conjugates, one or more non-cationic lipids, and one or more cationic lipids chosen from 1-{3,4-bis[(9Z,12Z)-octadeca-9,12-dienyloxy]butyl}piperidine, 4-{3,4-bis[(9Z,12Z)-octadeca-9,12-dienyloxy]butyl}morpholine, N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, 1-{3,4-bis[(9Z,12Z)-octadeca-9,12-dienyloxy]butyl}-4-phenylpiperazine, 1-{3,4-bis[(9Z,12Z)-octadeca-9,12-dienyloxy]butyl}-4-methylpiperazine, N-(2-methoxyethyl)-N-methyl-3,4-bis[(9Z,12Z)-octadeca-9,12-dienyloxy]butan-1-amine, 1-{3,4-bis[(9Z,12Z)-octadeca-9,12-dienyloxy]butyl}-4-(2-methoxyphenyl)piperazine, N-{3,4-bis[(9Z,12Z)-octadeca-9,12-dienyloxy]butyl}-N,N′,N′-trimethylethane-1,2-diamine, N-{3,4-bis[(9Z,12Z)-octadeca-9,12-dienyloxy]butyl}-N-methyl-N-(2-pyridin-2-ylethyl)amine, N-benzyl-N-{3,4-bis[(9Z,12Z)-octadeca-9,12-dienyloxy]butyl}-N-methylamine, N-{3,4-bis[(9Z,12Z)-octadeca-9,12-dienyloxy]butyl}-N-(4-fluorobenzyl)-N-methylamine, 1-{3,4-bis[(9Z,12Z)-octadeca-9,12-dienyloxy]butyl}-4-(2-fluorophenyl)piperazine, N-benzyl-N-{3,4-bis[(9Z,12Z)-octadeca-9,12-dienyloxy]butyl}-N-ethylamine, N-{3,4-bis[(9Z,12Z)-octadeca-9,12-dienyloxy]butyl}-N-ethyl-N′,N′-dimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, N,N-bis(2-methoxyethyl)-3,4-bis[(9Z,12Z)-octadeca-9,12-dienyloxy]butan-1-amine, 1-{3,4-bis[(9Z,12Z)-octadeca-9,12-dienyloxy]butyl}-4-methoxypiperidine, 1-{(3R)-3,4-bis[(9Z,12Z)-octadeca-9,12-dienyloxy]butyl}pyrrolidine, 1-{(3S)-3,4-bis[(9Z,12Z)-octadeca-9,12-dienyloxy]butyl}pyrrolidine, N-{(3R)-3,4-bis[(9Z,12Z)-octadeca-9,12-dienyloxy]butyl}-N,N-diethylamine, N-{(3S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 2-(2-{3,4-bis[(9Z,12Z)-octadeca-9,12-dienyloxy]butoxy}ethyl)-1-methylpyrrolidine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis[(9Z,12Z)-octadeca-9,12-dienyloxy]butoxy}ethyl)-N,N-dimethylamine, 4-(diethylamino)-2-[(9Z,12Z)-octadeca-9,12-dienoyloxy]butyl (9Z,12Z)-octadeca-9,12-dienoate, 1-(2-{3,4-bis[(9Z,12Z)-octadeca-9,12-dienyloxy]butoxy}ethyl)pyrrolidine, N,N-diethyl-N-(2-{2-[(8Z,11Z)-heptadeca-8,11-dienyl]-2-[(9Z,12Z)-octadeca-9,12-dienyl]-1,3-dioxolan-4-yl}ethyl)amine, 1-{[(9Z)-octadec-9-enoyloxy]methyl}-3-pyrrolidin-1-ylpropyl (9Z)-octadec-9-enoate, 1-{3,4-bis[(9Z)-octadec-9-enyloxy]butyl}pyrrolidine, 1-{[(5Z,8Z,11Z,14Z)-icosa-5,8,11,14-tetraenoyloxy]methyl}-3-pyrrolidin-1-ylpropyl (5Z,8Z,11Z,14Z)-icosa-5,8,11,14-tetraenoate, (3S)-3,4-bis[(9Z,12Z)-octadeca-9,12-dienyloxy]butyl 3-pyrrolidin-1-ylpropylcarbamate, 1-[3,4-bis(octadecyloxy)butyl]pyrrolidine, 1-[3,4-bis(hexadecyloxy)butyl]pyrrolidine, 1-{3,4-bis[(9E)-hexadec-9-enyloxy]butyl}pyrrolidine, 1-{3,4-bis[(9E)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N¹-{(3S)-3,4-bis[(9Z,12Z)-octadeca-9,12-dienyloxy]butyl}-N³,N³-diethyl-beta-alaninamide, N-{3,4-bis[(9Z,12Z)-octadeca-9,12-dienyloxy]butyl}-N-[3-(1H-imidazol-1-yl)propyl]amine, N-{3,4-bis[(9Z,12Z)-octadeca-9,12-dienyloxy]butyl}-N,N′,N′-trimethylpropane-1,3-diamine, 1-(1-{3,4-bis[(9Z,12Z)-octadeca-9,12-dienyloxy]butyl}pyrrolidin-3-yl)-1H-imidazole, N-{3,4-bis[(9Z,12Z)-octadeca-9,12-dienyloxy]butyl}-N-(3-pyrrolidin-1-ylpropyl)amine, N-{3,4-bis[(9Z,12Z)-octadeca-9,12-dienyloxy]butyl}-N′,N′-dimethylpropane-1,3-diamine, 1-{3,4-bis[(9Z,12Z)-octadeca-9,12-dienyloxy]butyl}azetidine, 1-{3,4-bis[(9Z,12Z)-octadeca-9,12-dienyloxy]butyl}-2-methylpyrrolidine, and 1-{3,4-bis[(9Z,12Z)-octadeca-9,12-dienyloxy]butyl}-2,5-dimethylpyrrolidine, effectively encapsulate nucleic acids, such as siRNA, with efficiencies from about 50-100%.

In still a further embodiment, functional CaBLES comprising one or more (PEG)-lipid conjugates, one or more non-cationic lipids, and one or more cationic lipids chosen from 1-{3,4-bis[(9Z,12Z)-octadeca-9,12-dienyloxy]butyl}piperidine, 4-{3,4-bis[(9Z,12Z)-octadeca-9,12-dienyloxy]butyl}morpholine, N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, 1-{3,4-bis[(9Z,12Z)-octadeca-9,12-dienyloxy]butyl}-4-phenylpiperazine, 1-{3,4-bis[(9Z,12Z)-octadeca-9,12-dienyloxy]butyl}-4-methylpiperazine, N-(2-methoxyethyl)-N-methyl-3,4-bis[(9Z,12Z)-octadeca-9,12-dienyloxy]butan-1-amine, 1-{3,4-bis[(9Z,12Z)-octadeca-9,12-dienyloxy]butyl}-4-(2-methoxyphenyl)piperazine, N-{3,4-bis[(9Z,12Z)-octadeca-9,12-dienyloxy]butyl}-N,N′,N′-trimethylethane-1,2-diamine, N-{3,4-bis[(9Z,12Z)-octadeca-9,12-dienyloxy]butyl}-N-methyl-N-(2-pyridin-2-ylethyl)amine, N-benzyl-N-{3,4-bis[(9Z,12Z)-octadeca-9,12-dienyloxy]butyl}-N-methylamine, N-{3,4-bis[(9Z,12Z)-octadeca-9,12-dienyloxy]butyl}-N-(4-fluorobenzyl)-N-methylamine, 1-{3,4-bis[(9Z,12Z)-octadeca-9,12-dienyloxy]butyl}-4-(2-fluorophenyl)piperazine, N-benzyl-N-{3,4-bis[(9Z,12Z)-octadeca-9,12-dienyloxy]butyl}-N-ethylamine, N-{3,4-bis[(9Z,12Z)-octadeca-9,12-dienyloxy]butyl}-N-ethyl-N′,N′-dimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, N,N-bis(2-methoxyethyl)-3,4-bis[(9Z,12Z)-octadeca-9,12-dienyloxy]butan-1-amine, 1-{3,4-bis[(9Z,12Z)-octadeca-9,12-dienyloxy]butyl}-4-methoxypiperidine, 1-{(3R)-3,4-bis[(9Z,12Z)-octadeca-9,12-dienyloxy]butyl}pyrrolidine, 1-{(3S)-3,4-bis[(9Z,12Z)-octadeca-9,12-dienyloxy]butyl}pyrrolidine, N-{(3R)-3,4-bis[(9Z,12Z)-octadeca-9,12-dienyloxy]butyl}-N,N-diethylamine, N-{(3S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 2-(2-{3,4-bis[(9Z,12Z)-octadeca-9,12-dienyloxy]butoxy}ethyl)-1-methylpyrrolidine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis[(9Z,12Z)-octadeca-9,12-dienyloxy]butoxy}ethyl)-N,N-dimethylamine, 4-(diethylamino)-2-[(9Z,12Z)-octadeca-9,12-dienoyloxy]butyl (9Z,12Z)-octadeca-9,12-dienoate, 1-(2-{3,4-bis[(9Z,12Z)-octadeca-9,12-dienyloxy]butoxy}ethyl) pyrrolidine, N,N-diethyl-N-(2-{2-[(8Z,11Z)-heptadeca-8,11-dienyl]-2-[(9Z,12Z)-octadeca-9,12-dienyl]-1,3-dioxolan-4-yl}ethyl)amine, 1-{[(9Z)-octadec-9-enoyloxy]methyl}-3-pyrrolidin-1-ylpropyl (9Z)-octadec-9-enoate, 1-{3,4-bis[(9Z)-octadec-9-enyloxy]butyl}pyrrolidine, 1-{[(5Z,8Z,11Z,14Z)-icosa-5,8,11,14-tetraenoyloxy]methyl}-3-pyrrolidin-1-ylpropyl (5Z,8Z,11Z,14Z)-icosa-5,8,11,14-tetraenoate, (3S)-3,4-bis[(9Z,12Z)-octadeca-9,12-dienyloxy]butyl 3-pyrrolidin-1-ylpropylcarbamate, 1-[3,4-bis(octadecyloxy)butyl]pyrrolidine, 1-[3,4-bis(hexadecyloxy)butyl]pyrrolidine, 1-{3,4-bis[(9E)-hexadec-9-enyloxy]butyl}pyrrolidine, 1-{3,4-bis[(9E)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N¹-{(3S)-3,4-bis[(9Z,12Z)-octadeca-9,12-dienyloxy]butyl}-N³,N³-diethyl-beta-alaninamide, N-{3,4-bis[(9Z, 12Z)-octadeca-9,12-dienyloxy]butyl}-N-[3-(1H-imidazol-1-yl)propyl]amine, N-{3,4-bis[(9Z, 12Z)-octadeca-9,12-dienyloxy]butyl}-N,N′,N′-trimethylpropane-1,3-diamine, 1-(1-{3,4-bis[(9Z,12Z)-octadeca-9,12-dienyloxy]butyl}pyrrolidin-3-yl)-1H-imidazole, N-{3,4-bis[(9Z,12Z)-octadeca-9,12-dienyloxy]butyl}-N-(3-pyrrolidin-1-ylpropyl)amine, N-{3,4-bis[(9Z,12Z)-octadeca-9,12-dienyloxy]butyl}-N′,N′-dimethylpropane-1,3-diamine, 1-{3,4-bis[(9Z,12Z)-octadeca-9,12-dienyloxy]butyl}azetidine, 1-{3,4-bis[(9Z,12Z)-octadeca-9,12-dienyloxy]butyl}-2-methylpyrrolidine, and 1-{3,4-bis[(9Z,12Z)-octadeca-9,12-dienyloxy]butyl}-2,5-dimethylpyrrolidine, effectively encapsulate nucleic acids, such as siRNA, with efficiencies from about 80-100%.

A further embodiment pertains to examples of non-cationic lipids that are useful for the practice of this invention which include, but are not limited to, cholesterol, cholesterol sulfate, ceramide, sphingomyelin, lecithin, sphingomyelin, egg sphingomyelin, milk sphingomyelin; egg phosphatidylcholine, hydrogenated egg phosphatidylcholine, hydrogenated soybean phosphatidylethanolamine, egg phosphatidylethanolamine, hydrogenated soybean phosphatidylcholine, soybean phosphatidylcholine, 1,2-dilauroyl-sn-glycerol, 1,2-dimyristoyl-sn-glycerol, 1,2-dipalmitoyl-sn-glycerol, 1,2-distearoyl-sn-glycerol, 1,2-dilauroyl-sn-glycero-3-phosphatidic acid, 1,2-dimyristoyl-sn-glycero-3-phosphatidic acid, 1,2-dipalmitoyl-sn-glycero-3-phosphatidic acid, 1,2-distearoyl-sn-glycero-3-phosphatidic acid, 1,2-diarachidoyl-sn-glycero-3-phosphocholine, 1,2-dilauroyl-sn-glycero-3-phosphocholine, 1,2-dimyristoyl-sn-glycero-3-phosphocholine, dioleoylphosphatidylcholine, 1,2-dierucoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-stearoyl-sn-glycero-3-phosphocholine, 1-stearoyl-2-myristoyl-sn-glycero-3-phosphocholine, 1-stearoyl-2-palmitoyl-sn-glycero-3-phosphocholine, 1-myristoyl-2-oleoyl-sn-glycero-3-phosphocholine, 1-palmitoyl-2-oleoyl-sn-glycero-3-phosphocholine; 1-stearoyl-2-oleoyl-sn-glycero-3-phosphocholine, 1-myristoyl-2-lyso-sn-glycero-3-phosphocholine, 1-palmitoyl-2-lyso-sn-glycero-3-phosphocholine, 1-stearoyl-2-lyso-sn-glycero-3-phosphocholine, 1,2-dipalmitoyl-sn-glycero-O-ethyl-3-phosphocholine, 1,2-dipalmitoyl-sn-glycero-3-phosphocholine; 1,2-distearoyl-sn-glycero-3-phosphocholine; 1-palmitoyl-2-linoleoyl-sn-glycero-3-phosphocholine, dioleoylphosphatidylethanolamine, palmitoyloleoyl-phosphatidylethanolamine, dioleoylphosphatidylglycerol,1,2-dilauroyl-sn-glycero-3-phosphoethanolamine, 1,2-dimyristoyl-sn-glycero-3-phosphoethanolamine, 1,2-dipalmitoyl-sn-glycero-3-phosphoethanolamine, 1,2-distearoyl-sn-glycero-3-phosphoethanolamine, 1,2-dioleoyl-sn-glycero-3-phosphoethanolamine, 1,2-dilauroyl-sn-glycero-3-phosphoglycerol, 1,2-dimyristoyl-sn-glycero-3-phosphoglycerol,1,2-dimyristoyl-sn-glycero-3-phospho-sn-1-glycerol, 1,2-dipalmitoyl-sn-glycero-3-phosphoglycerol, 1,2-distearoyl-sn-glycero-3-phosphoglycero, 1,2-distearoyl-sn-glycero-3-phospho-sn-1-glycerol, 1-palmitoyl-2-oleoyl-sn-glycero-3-phosphoglycerol, 1-palmitoyl-2-oleoyl-sn-glycero-3-phosphoglycerol, 1,2-dipalmitoyl-sn-glycero-3-phospho-L-serine, 1,2-dimyristoyl-sn-glycero-3-phospho-L-serine, 1,2-dipalmitoyl-sn-glycero-3-phospho-L-serine, 1,2-distearoyl-sn-glycero-3-phospho-L-serine, 1,2-dioleoyl-sn-glycero-3-phospho-L-serine, and 1-palmitoyl-2-oleoyl-sn-glycero-3-phospho-L-serine or a mixture thereof.

A further embodiment pertains to examples of PEG-lipid conjugates that are useful for the practice of this invention which include, but are not limited to, 2-(tetradecyloxy)-1-((tetradecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate, 2-(hexadecyloxy)-1-((hexadecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate, 2-(octadecyloxy)-1-((octadecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate, 2-2,5,8,11,14,17,20,23,26,29,32,35,38,41,44,47,50,53,56,59,62,65,68,71,74,77,80,83,86,89,92, 94,97,100,103,106,109,112,115,118,121,124,127,130,133,136-hexatetracontaoxaoctatriacontahectanamidopropane-1,3-diyl ditetradecanoate, 2-2,5,8,11,14,17,20,23,26,29,32,35,38,41,44,47,50,53,56,59,62,65,68,71,74,77,80,83,86,89,92, 94,97,100,103,106,109,112,115,118,121,124,127,130,133,136-hexatetracontaoxaoctatriacontahectanamidopropane-1,3-diyl dipalmitate, 2-2,5,8,11,14,17,20,23,26,29,32,35,38,41,44,47,50,53,56,59,62,65,68,71,74,77,80,83,86,89,92, 94,97,100,103,106,109,112,115,118,121,124,127,130,133,136-hexatetracontaoxaoctatriacontahectanamidopropane-1,3-diyl distearate, N-(2-(hexadecyloxy)-1-((hexadecyloxy)methyl)ethyl)-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-(2-(tetradecyloxy)-1-((tetradecyloxy)methyl)ethyl)-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide,N-(2-(octadecyloxy)-1-((octadecyloxy)methyl)ethyl)-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, 6-oxo-2-(tetradecanoyloxy)-8,11,14,17,20,23,26,29,32,35,38,41,44,47,50,53,56,59,62,65,68,71,74,77,80,83,86,89,92,95,9 8,101,104,107,110,113,116,119,122,125,128,131,134,137,140,143-hexatetracontaoxa-5-azatetratetracontahect-1-yl myristate, N-[3,4-bis(tetradecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-[3,4-bis(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, 3,7,11,15,19,23,27,31,35,39,43,47,51,55,59,63,67,71,75,79,83,87,91,95,99,103,107,111,115, 119,123,127,131,135,139,143,147,151,155,159,163,167,171,175,179,182-hexatetracontaoxatrioctacontahect-1-yl 3,4-bis(tetradecyloxy)butylcarbamate, 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-yl 3,4-bis(hexadecyloxy)butylcarbamate, 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-yl 3,4-bis(octadecyloxy)butylcarbamate, N-[3,4-bis(hexadecyloxy)butyl]-N′-3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylsuccinamide, 6-oxo-2-(tetradecanoyloxy)-7,10,13,16,19,22,25,28,31,34,37,40,43,46,49,52,55,58,61,64,67,70,73,76,79,82,85,88,91,94,9 7,100,103,106,109,112,115,118,121,124,127,130,133,136,139,142,145-heptatetracontaoxa-5-azahexatetracontahect-1-yl myristate, 6-oxo-2-(palmitoyloxy)-7,10,13,16,19,22,25,28,31,34,37,40,43,46,49,52,55,58,61,64,67,70,73,76,79,82,85,88,91,94,9 7,100,103,106,109,112,115,118,121,124,127,130,133,136,139,142,145-heptatetracontaoxa-5-azahexatetracontahect-1-yl palmitate, 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-yl 4-{[3,4-bis(hexadecyloxy)butyl]amino }-4-oxobutanoate, 6-oxo-2-(palmitoyloxy)-8,11,14,17,20,23,26,29,32,35,38,41,44,47,50,53,56,59,62,65,68,71,74,77,80,83,86,89,92,95,9 8,101,104,107,110,113,116,119,122,125,128,131,134,137,140,143-hexatetracontaoxa-5-azatetratetracontahect-1-yl palmitate, N-[4-(decyloxy)-3-(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-[3,4-bis(decyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-[3,4-bis(hexadecyloxy)butyl]-2,5,8,11,14,17,20,23,26,29,32,35,38,41,44,47,50,53,56,59,62,65,68-tricosaoxaheptacontan-70-amide, N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143,146,149,152,155,158, 161,164,167,170,173,176,179,182,185,188,191,194,197,200,203,206,209,212,215,218,221,2 24,227,230,233,236,239,242,245,248,251,254,257,260,263,266,269,272,275,278,281,284,28 7,290,293,296,299,302,305,308,311,314,317,320,323,326,329,332,335,338-113oxa340n-340-amide, N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, 1,2-distearoyl-sn-glycerol-methoxypolyethyleneglycol-750, 1,2-dimyristoyl-sn-glycerol-methoxypolyethyleneglycol-750, 1,2-dipalmitoyl-sn-glycerol-methoxypolyethyleneglycol-750, poly(oxy-1,2-ethanediyl)-2000-α-(3β)-cholest-5-en-3-yl-omega-hydroxy, 1,2-dipalmitoyl-sn-glycerol-methoxypolyethyleneglycol-5000, poly(oxy-1,2-ethanediyl)-5000-α-(3β)-cholest-5-en-3-yl-omega-hydroxy, (2S,3R,E)-3-hydroxy-2-stearamidooctadec-4-enyl polyethyleneglycol-2000 methyl ether succinate, (2S,3R,E)-3-hydroxy-2-icosanamidooctadec-4-enyl polyethyleneglycol-2000 methyl ether succinate, N-(2,3-dimyristyloxypropyl)carbamate polyethyleneglycol-2000 methyl ether, N-(carbonylmethoxypolyethyleneglycol-750)-1,2-dimyristoyl-sn-glycero-phosphatidylethanolamine, N-(carbonyl-methoxypolyethyleneglycol-750)-1,2-distearoyl-sn-glycero-3-phosphoethanolamine, N-(carbonyl-methoxypolyethyleneglycol-750)-1,2-dipalmitoyl-sn-glycero-3-phosphoethanolamine, N-(carbonyl-methoxypolyethyleneglycol-2000)-1,2-dimyristoyl-sn-glycero-3-phosphoethanolamine, N-(carbonyl-methoxypolyethyleneglycol-2000)-1,2-dipalmitoyl-sn-glycero-3-phosphoethanolamine, N-(carbonyl-methoxypolyethyleneglycol-2000)-1,2-distearoyl-sn-glycero-3-phosphoethanolamine, N-(carbonyl-methoxypolyethyleneglycol-2000)-dioleoyl-phosphatidylethanolamine, 1,2-distearoyl-sn-glycerol-methoxypolyethyleneglycol-2000, 1,2-dimyristoyl-sn-glycerol-methoxypolyethyleneglycol-2000, 1,2-dipalmitoyl-sn-glycerol-methoxypolyethyleneglycol-2000, mPEG-2000-cholesterol, octanoyl-mPEG-2000-ceramide, palmitoyl-mPEG-2000-ceramide, N-(carbonyl-methoxypolyethyleneglycol-5000)-1,2-dimyristoyl-sn-glycero-3-phosphoethanolamine, N-(carbonyl-methoxypolyethyleneglycol-5000)-1,2-dipalmitoyl-sn-glycero-3-phosphoethanolamine, N-(carbonyl-methoxypolyethyleneglycol-5000)-1,2-distearoyl-sn-glycero-3-phosphoethanolamine, 1,2-dimyristoyl-sn-glycerol-methoxypolyethyleneglycol-5000, 1,2-dipalmitoyl-sn-glycerol-methoxypolyethyleneglycol-5000, 1,2-distearoyl-sn-glycerol-methoxypolyethyleneglycol-5000, mPEG-5000-cholesterol, octanoyl-mPEG-5000-ceramide, palmitoyl-mPEG-5000-ceramide and mixtures thereof.

PEG-lipid conjugates are described in, e.g., U.S. App. No. 61/095,748, which was filed on Sep. 10, 2008 and is incorporated herein by reference.

PEG-lipid conjugates are described in, e.g., U.S. App. No. 61/095,769, which was filed on Sep. 10, 2008 and is incorporated herein by reference.

A still further embodiment pertains to combinations of polyethylene glycol (PEG)-lipid conjugates which are useful for the practice of this invention, wherein two PEG-lipid conjugates are chosen from N-(carbonyl-methoxypolyethyleneglycol-2000)-1,2-dimyristoyl-sn-glycero-3-phosphoethanolamine , N-(carbonyl-methoxypolyethyleneglycol-2000)-1,2-dipalmitoyl-sn-glycero-3-phosphoethanolamine, N-(carbonyl-methoxypolyethyleneglycol-2000)-1,2-distearoyl-sn-glycero-3-phosphoethanolamine, 1,2-distearoyl-sn-glycerol-methoxypolyethyleneglycol-2000, 1,2-dimyristoyl-sn-glycerol-methoxypolyethyleneglycol-2000, 1,2-dipalmitoyl-sn-glycerol-methoxypolyethyleneglycol-2000, and N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide.

A still further embodiment pertains to combinations of polyethylene glycol (PEG)-lipid conjugates which are useful for the practice of this invention, wherein at least one of the PEG-lipid conjugates is chosen from N-(carbonyl-methoxypolyethyleneglycol-2000)-1,2-dimyristoyl-sn-glycero-3-phosphoethanolamine, N-(carbonyl-methoxypolyethyleneglycol-2000)-1,2-dipalmitoyl-sn-glycero-3-phosphoethanolamine, N-(carbonyl-methoxypolyethyleneglycol-2000)-1,2-distearoyl-sn-glycero-3-phosphoethanolamine, 1,2-distearoyl-sn-glycerol-methoxypolyethyleneglycol-2000, 1,2-dimyristoyl-sn-glycerol-methoxypolyethyleneglycol-2000, 1,2-dipalmitoyl-sn-glycerol-methoxypolyethyleneglycol-2000, and N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide.

A still further embodiment pertains to combinations of polyethylene glycol (PEG)-lipid conjugates which are useful for the practice of this invention and include 1,2-dimyristoyl-sn-glycerol-methoxypolyethyleneglycol-2000 and N-(carbonyl-methoxypolyethyleneglycol-2000)-1,2-distearoyl-sn-glycero-3-phosphoethanolamine, 1,2-distearoyl-sn-glycerol-methoxypolyethyleneglycol-2000 and N-(carbonyl-methoxypolyethyleneglycol-2000)-1,2-dimyristoyl-sn-glycero-3-phosphoethanolamine, N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine, N-(carbonyl-methoxypolyethyleneglycol-2000)-1,2-distearoyl-sn-glycero-3-phosphoethanolamine and N-(carbonyl-methoxypolyethyleneglycol-2000)-1,2-dipalmitoyl-sn-glycero-3-phosphoethanolamine, 1,2-dimyristoyl-sn-glycerol-methoxypolyethyleneglycol-2000 and 1,2-dipalmitoyl-sn-glycerol-methoxypolyethyleneglycol-2000, 1,2-distearoyl-sn-glycerol-methoxypolyethyleneglycol-2000 and 1,2-dimyristoyl-sn-glycerol-methoxypolyethyleneglycol-2000; N-(carbonyl-methoxypolyethyleneglycol-2000)-1,2-dimyristoyl-sn-glycero-3-phosphoethanolamine and N-(carbonyl-methoxypolyethyleneglycol-2000)-1,2-distearoyl-sn-glycero-3-phosphoethanolamine, N-(carbonyl-methoxypolyethyleneglycol-2000)-1,2-dimyristoyl-sn-glycero-3-phosphoethanolamine and N-(carbonyl-methoxypolyethyleneglycol-2000)-1,2-dipalmitoyl-sn-glycero-3-phosphoethanolamine, and N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide and 1,2-distearoyl-sn-glycerol-methoxypolyethyleneglycol-2000.

In still a further embodiment, the cationic lipids of the CaBLES and Lipid-Based Particles comprises about 2 to about 60 weight/weight percent of total lipid in the particle.

In still a further embodiment, the non-cationic lipids of the Cables and Lipid-Based Particles comprises about 5 to about 90 weight/weight percent of total lipid in the particle.

In still a further embodiment, the PEG-lipid conjugates of the CaBLES and Lipid-Based Particles comprises from 0.1 to about 20 weight/weight percent of total lipid in the particle.

Methods of Treatment and Methods ofMaking Lipid-Based Particles

Still another embodiment pertains to a method of treating cancer in a mammal comprising administering thereto a Lipid-Based Particle.

Still another embodiment comprises methods of treating cancer in a mammal comprising administering thereto a Lipid-Based Particle comprising one or more cationic lipids having Formula (I)

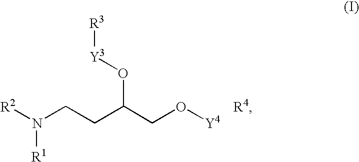

### Formula (II)

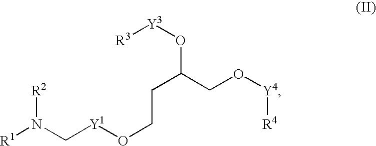

### Formula (III)

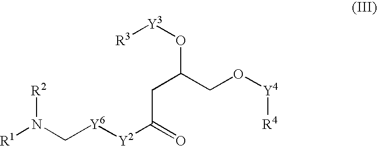

or

### Formula (IV)

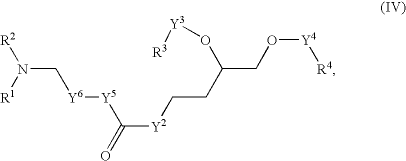

wherein Y¹ is C₁-C₆ alkylene;

Y² is CH₂, NH or O;

Y³ is a bond or C(O);

Y⁴ is a bond or C(O);

Y⁵ is CH₂, NH or O;

Y⁶ is a bond or C₁-C₆ alkylene;

R¹ and R² are independently H, cycloalkyl, cycloalkenyl or R⁵; or

R¹ and R², together with the nitrogen to which they are attached, are heterocycloalkyl or heteroaryl;

one of R³ and R⁴ is H, and the other is C₁₄-C₂₀-alkenyl, or C₁₄-C₂₀-alkyl; or R³ and R⁴ are independently selected C₁₄-C₂₀-alkenyl, or C₁₄-C₂₀-alkyl; or

R³ and R⁴ together are CR²⁰R²¹, wherein R²⁰ is H and R²¹ is C₁₄-C₂₀-alkenyl, C₁₄-C₂₀-alkyl, or CH₂O—C₁₄-C₂₀-alkenyl; or R²⁰ and R²¹ are independently selected C₁₄-C₂₀-alkenyl, C₁₄-C₂₀-alkyl, or CH₂O—C₁₄-C₂₀-alkenyl;

R⁵ is alkyl, which is unsubstituted or substituted with one or more R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, C(N)NH₂, C(N)NHR⁶, C(N)N(R⁶)₂, CNOH, CNOCH₃, OH, (O), CN, N₃, NO₂, CF₃, CF₂CF₃, OCF₃, OCF₂CF₃, F, Cl, Br or I;

R⁶ is R⁷, R⁸, R⁹, or R¹⁰;

R⁷ is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁸ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R⁹ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁰ is alkyl, alkenyl or alkynyl;

wherein each foregoing cyclic moiety is independently unsubstituted or substituted with one or more R¹¹, OR¹¹, SR¹¹, S(O)R¹¹, SO₂R¹¹, C(O)R¹¹, CO(O)R¹¹, OC(O)R¹¹, OC(O)OR¹¹, NH₂, NHR¹¹, N(R¹¹)₂, NHC(O)R¹¹, NR¹¹C(O)R¹¹, NHS(O)₂R¹¹, NR¹¹S(O)₂R¹¹, NHC(O)OR¹¹, NR¹¹C(O)OR¹¹, NHC(O)NH₂, NHC(O)NHR¹¹, NHC(O)N(R¹¹), NR¹¹C(O)NHR¹¹, NR¹¹C(O)N(R¹¹), C(O)NH₂, C(O)NHR¹¹, C(O)N(R¹¹)₂, C(O)NHOH, C(O)NHOR¹¹, C(O)NHSO₂R¹¹, C(O)NR¹¹SO₂R¹¹, SO₂NH₂, SO₂NHR¹¹,SO₂N(R¹¹)₂, C(O)H, C(O)OH, C(N)NH₂, C(N)NHR¹¹, C(N)N(R¹¹)₂, CNOH, CNOCH₃, OH, (O), CN, N₃, NO₂, CF₃, CF₂CF₃, OCF₃,OCF₂CF₃, F, Cl, Br or I;

R¹¹ is R¹², R¹³, R¹⁴, or R¹⁵;

R¹² is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹³ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁴ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;

R¹⁵is alkyl, alkenyl or alkynyl, each of which is unsubstituted or substituted with one or two of independently selected R¹⁶, OR^{16E}, SR¹⁶, S(O)₂R¹⁶, C(O)OH, NH₂, NHR¹⁶ N(R¹⁶)₂, C(O)R¹⁶, C(O)NH₂, C(O)NHR¹⁶, C(O)N(R¹⁶)₂, NHC(O)R¹⁶, NR¹⁶C(O)R¹⁶, NHC(O)OR¹⁶, NR¹⁶C(O)OR¹⁶, OH, F, Cl, Br or I;

R¹⁶ is alkyl, alkenyl, alkynyl, or R¹⁷;

R¹⁷ is phenyl, heteroaryl, cycloalkyl, cycloalkenyl, heterocycloalkyl or heterocycloalkenyl;

wherein R¹², R¹³, R¹⁴, and R¹⁷ are independently unsubstituted or substituted with one or more R¹⁸, OR¹⁸, SR¹⁸, S(O)R¹⁸, SO₂R¹⁸, C(O)R¹⁸, CO(O)R¹⁸, OC(O)R¹⁸, OC(O)OR¹⁸, NH₂, NHR¹⁸, N(R¹⁸)₂, NHC(O)R¹⁸, NR¹⁸C(O)R¹⁸, NHS(O)₂R¹⁸, NR¹⁸S(O)₂R¹⁸, NHC(O)OR¹⁸, NR¹⁸C(O)OR¹⁸, NHC(O)NH₂, NHC(O)NHR¹⁸, NHC(O)N(R¹⁸)₂, NR¹⁸C(O)NHR¹⁸, NR¹⁸C(O)N(R¹⁸)₂, C(O)NH₂, C(O)NHR¹⁸, C(O)N(R¹⁸)₂, C(O)NHOH, C(O)NHOR¹⁸, C(O)NHSO₂R¹⁸, C(O)NR¹⁸SO₂R¹⁸, SO₂NH₂, SO₂NHR¹⁸, SO₂N(R¹⁸)₂, C(O)H, C(O)OH, C(N)NH₂, C(N)NHR¹⁸, C(N)N(R¹⁸)₂, CNOH, CNOCH₃, OH, (O), CN, N₃, NO₂, CF₃, CF₂CF₃, OCF₃, OCF₂CF₃, F, Cl, Br or I;

R¹⁸ is alkyl, alkenyl, alkynyl, phenyl, heteroaryl, cycloalkyl, cycloalkenyl, heterocycloalkyl or heterocycloalkenyl; and

one or more non-cationic lipids, one or more polyethylene glycol-lipid conjugates and one or more therapeutic agents.

A further embodiment pertains to a method of making Lipid-Based Particles, comprising: (a) mixing the cationic lipid(s), the non-cationic lipid(s) and the PEG-lipid conjugate(s); (b) adding the mixture of step (a) to one or more therapeutic agents; and (c) separating and purifying resulting suspension of step (b).

A further embodiment pertains to a method of making Lipid-Based Particles wherein the mixture of step (a) and one or more said therapeutic agents are warmed to about 60° C. prior to the addition of the mixture of step (a) to one or more therapeutic agents via needle injection.

### Pharmaceutical Compositions and Methods of Administration

Therapeutically effective amounts of Lipid-Based Particles of this invention depend on recipient of treatment, disease treated and severity thereof, composition comprising it, time of administration, route of administration, duration of treatment, potency, rate of clearance and whether or not another drug is co-administered. The amount of Lipid-Based Particles of this invention used to make compositions to be administered daily to a patient in a single dose or in divided doses is from about 0.001 to about 200 mg/kg body weight. Single dose compositions contain these amounts or a combination of submultiples thereof.

One embodiment pertains to a pharmaceutical composition comprising one or more (PEG)-lipid conjugates, one or more non-cationic lipids, one or more cationic lipids of Formula I, II, III, or IV, one or more therapeutic agents, and a pharmaceutically acceptable excipient.

Lipid-Based Particles of this invention may be administered, for example, bucally, ophthalmically, orally, osmotically, parenterally (intramuscularly, intraperintoneally intrasternally, intravenously, subcutaneously), rectally, topically, transdermally, vaginally and intraarterially as well as by intraarticular injection, infusion, and placement in the body, such as, for example, the vasculature.

Lipid-Based Particles may be administered with or without an excipient. Excipients include, but are not limited to, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising Lipid-Based Particles to be administered orally include, but are not limited to, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, mixtures thereof and the like. Excipients for preparation of compositions comprising a compound having formula (I) to be administered ophthalmically or orally include, but are not limited to, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, mixtures thereof and the like. Excipients for preparation of compositions comprising a compound having formula (I) to be administered osmotically include, but are not limited to, chlorofluorohydrocarbons, ethanol, water, mixtures thereof and the like. Excipients for preparation of compositions comprising a compound having formula (I) to be administered parenterally include, but are not limited to, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, mixtures thereof and the like. Excipients for preparation of compositions comprising a compound having formula (I) to be administered rectally or vaginally include, but are not limited to, cocoa butter, polyethylene glycol, wax, mixtures thereof and the like.

The pharmaceutical composition and the method of the present invention may further comprise other therapeutically active compounds as noted herein which are usually applied in the treatment of the above-mentioned pathological conditions.

### Combination Therapy

The present invention further provides methods of using a compound, formulation, or composition of the invention in combination with one or more additional active agents.

Lipid-Based Particles are expected to be useful when used with: alkylating agents, angiogenesis inhibitors, antibodies, antimetabolites, antimitotics, antiproliferatives, aurora kinase inhibitors, apoptosis promoters (for example, Bcl-xL, Bcl-w and Bfl-1) inhibitors, Bcr-Abl kinase inhibitors, BiTE (Bi-Specific T cell Engager) antibodies, biologic response modifiers, cyclin-dependent kinase inhibitors, cell cycle inhibitors, cyclooxygenase-2 inhibitors, DVD's, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of apoptosis proteins (IAP's) intercalating antibiotics, kinase inhibitors, mammalian target of rapamycin inhibitors, microRNA's mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, proteosome inhibitors, purine analogs, pyrimidine analogs, receptor tyrosine kinase inhibitors, retinoids/deltoids plant alkaloids, small inhibitory ribonucleic acids (siRNA's), topoisomerase inhibitors, combinations thereof and the like.

A BiTE antibody is a bi-specific antibody that directs T-cells to attach cancer cells by simultaneously binding the two cells. The T-cell then attacks the target cancer cell. Exemplary BiTE antibodies include adecatumumab (Micromet MT201), blinatumomab (Micromet MT103) and the like.

SiRNA's are molecules having endogenous RNA bases or chemically modified nucleotides. The modifications shall not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides or a combination thereof The siRNA can have varying lengths (10-200 bps) and structures (hairpins, single/double strands, bulges, nicks/gaps, mismatches) and processed in the cell to provide active gene silencing. In certain embodiments, a double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Multivalent binding proteins are binding proteins comprising two or more antigen binding sites. The multivalent binding protein is preferably engineered to have the three or more antigen binding sites and is generally not a naturally occurring antibody. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific, i.e., capable of binding one antigen or multispecific, i.e., capable of binding two or more antigens. DVD binding proteins comprising two heavy chain DVD polypeptides and two light chain DVD polypeptides are referred to as DVD Ig. Each half of a DVD Ig comprises a heavy chain DVD polypeptide, a light chain DVD polypeptide, and two antigen binding sites. Each binding site comprises a heavy chain variable domain and a light chain variable domain with a total of 6 CDRs involved in antigen binding per antigen binding site.

Alkylating agents include altretamine, AMD-473, AP-5280, apaziquone, bendamustine, brostallicin, busulfan, carboquone, carmustine (BCNU), chlorambucil, CLORETAZINE® (laromustine, VNP 40101M), cyclophosphamide, decarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, TREANDA® (bendamustine), treosulfan, rofosfamide and the like.

Angiogenesis inhibitors include endothelial-specific receptor tyrosine kinase (Tie-2) inhibitors, epidermal growth factor receptor (EGFR) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, vascular endothelial growth factor receptor tyrosine kinase (VEGFR) inhibitors and the like.

Antimetabolites include ALIMTA® (metrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflomithine, EICAR (5-ethynyl-1-β-D-ribofuranosylimidazole-4-carboxamide), enocitabine, ethnylcytidine, fludarabine, 5-fluorouracil alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosfate, pelitrexol, pentostatin, raltitrexed, Ribavirin, triapine, trimetrexate, S-1, tiazofurin, tegafur, TS-1, vidarabine, UFT and the like.

Bcl-2 proteins inhibitors include AT-101 ((−)gossypol), GENASENSE® (G3139 or oblimersen (Bcl-2-targeting antisense oligonucleotide)), IPI-194, IPI-565, N-(4-(4-((4′-chloro(1,1 ′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide) (ABT-737), N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide (ABT-263), GX-070 (obatoclax) and the like.

Bcr-Abl kinase inhibitors include DASATINIB® (BMS-354825), GLEEVEC® (imatinib) and the like.

CDK inhibitors include AZD-5438, BMI-1040, BMS-032, BMS-387, CVT-2584,

flavopyridol, GPC-286199, MCS-5A, PD0332991, PHA-690509, seliciclib (CYC-202, R-roscovitine), ZK-304709 and the like.

COX-2 inhibitors include ABT-963, ARCOXIA® (etoricoxib), BEXTRA® (valdecoxib), BMS347070, CELEBREX® (celecoxib), COX-189 (lumiracoxib), CT-3, DERAMAXX® (deracoxib), JTE-522, 4-methyl-2-(3,4-dimethylphenyl)-1-(4-sulfamoylphenyl-1H-pyrrole), MK-663 (etoricoxib), NS-398, parecoxib, RS-57067, SC-58125, SD-8381, SVT-2016, S-2474, T-614, VIOXX® (rofecoxib) and the like.

EGFR inhibitors include ABX-EGF, anti-EGFR immunoliposomes, EGF-vaccine, EMD-7200, ERBITUX® (cetuximab), HR3, IgA antibodies, IRESSA® (gefitinib), TARCEVA® (erlotinib or OSI-774), TP-38, EGFR fusion protein, TYKERB® (lapatinib) and the like.

ErbB2 receptor inhibitors include CP-724-714, CI-1033 (canertinib), HERCEPTIN® (trastuzumab), TYKERB® (lapatinib), OMNITARG® (2C4, petuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her2IgG3, AS HER2 trifunctional bispecfic antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17-AAG-nab, 17-AAG, CNF-101, CNF-1010, CNF-2024, 17-DMAG, geldanamycin, IPI-504, KOS-953, MYCOGRAB® (human recombinant antibody to HSP-90), NCS-683664, PU24FCl, PU-3, radicicol, SNX-2112, STA-9090 VER49009 and the like.

Inhibitors of apoptosis proteins include ApoMab (a fully human affinity-matured IgG1 monoclonal antibody), antibodies that target TRAIL or death receptors (e.g., pro-apoptotic receptor agonists DR4 and DR5), conatumumab, ETR2-STO1, GDC0145, (lexatumumab), HGS-1029, LBY-135, PRO-1762 and tratuzumab.

MEK inhibitors include ARRY-142886, ARRY-438162 PD-325901, PD-98059 and the like.

mTOR inhibitors include AP-23573, CCI-779, everolimus, RAD-001, rapamycin, temsirolimus and the like.

Non-steroidal anti-inflammatory drugs include AMIGESIC® (salsalate), DOLOBID® (diflunisal), MOTRIN® (ibuprofen), ORUDIS® (ketoprofen), RELAFEN® (nabumetone), FELDENE® (piroxicam), ibuprofen cream, ALEVE® (naproxen) and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), DAYPRO® (oxaprozin) and the like.

PDGFR inhibitors include C-451, CP-673, CP-868596 and the like.

Platinum chemotherapeutics include cisplatin, ELOXATIN® (oxaliplatin) eptaplatin, lobaplatin, nedaplatin, PARAPLATIN® (carboplatin), satraplatin and the like.

Polo-like kinase inhibitors include BI-2536 and the like.

Thrombospondin analogs include ABT-510, ABT-567, TSP-1 and the like.

VEGFR inhibitors include AVASTIN® (bevacizumab), ABT-869, AEE-788, ANGIOZYME™ (a ribozyme that inhibits angiogenesis (Ribozyme Pharmaceuticals (Boulder, Colo.) and Chiron, (Emeryville, Calif.)), axitinib (AG-13736), AZD-2171, CP-547,632, IM-862, MACUGEN (pegaptamib), NEXAVAR® (sorafenib, BAY⁴³-9006), pazopanib (GW-786034), vatalanib (PTK-787, ZK-222584), SUTENT® (sunitinib, SU-11248), VEGF trap, ZACTIMA™ (vandetanib, ZD-6474) and the like.

Antibiotics include intercalating antibiotics aclarubicin, actinomycin D, amrubicin, annamycin, adriamycin, BLENOXANE® (bleomycin), daunorubicin, CAELYX® or MYOCET® (liposomal doxorubicin), elsamitrucin, epirbucin, glarbuicin, ZAVEDOS® (idarubicin), mitomycin C, nemorubicin, neocarzinostatin, peplomycin, pirarubicin, rebeccamycin, stimalamer, streptozocin, VALSTAR® (valrubicin), zinostatin and the like.

Topoisomerase inhibitors include aclarubicin, 9-aminocamptothecin, amonafide, amsacrine, becatecarin, belotecan, BN-80915, CAMPTOSAR® (irinotecan hydrochloride), camptothecin, CARDIOXANE® (dexrazoxine), diflomotecan, edotecarin, ELLENCE® or PHARMORUBICIN® (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include AVASTIN® (bevacizumab), CD40-specific antibodies, chTNT-1/B, denosumab, ERBITUX® (cetuximab), HUMAX-CD4® (zanolimumab), IGF1R-specific antibodies, lintuzumab, PANOREX® (edrecolomab), RENCAREX® (WX G250), RITUXAN® (rituximab), ticilimumab, trastuzimab and and the like.

Hormonal therapies include ARIMIDEX® (anastrozole), AROMASIN® (exemestane), arzoxifene, CASODEX® (bicalutamide), CETROTIDE® (cetrorelix), degarelix, deslorelin, DESOPAN® (trilostane), dexamethasone, DROGENIL®, (flutamide), EVISTA® (raloxifene), AFEMA™ (fadrozole), FARESTON® (toremifene), FASLODEX® (fulvestrant), FEMARA® (letrozole), formestane, glucocorticoids, HECTOROL® (doxercalciferol), RENAGEL® (sevelamer carbonate), lasofoxifene, leuprolide acetate, MEGACE® (megesterol), MIFEPREX® (mifepristone), NILANDRON™ (nilutamide), NOLVADEX® (tamoxifen citrate), PLENAXIS™ (abarelix), prednisone, PROPECIA® (finasteride), rilostane, SUPREFACT® (buserelin), TRELSTAR® (luteinizing hormone releasing hormone (LHRH)), VANTAS® (Histrelin implant), VETORYL® (trilostane or modrastane), ZOLADEX® (fosrelin, goserelin) and the like.

Deltoids and retinoids include seocalcitol (EB1089, CB1093), lexacalcitrol (KH1060), fenretinide, PANRETIN® (aliretinoin), ATRAGEN® (liposomal tretinoin), TARGRETIN® (bexarotene), LGD-1550 and the like.

PARP inhibitors include ABT-888, olaparib, KU-59436, AZD-2281, AG-014699, BSI-201, BGP-15, INO-1001, ONO-2231 and the like.

Plant alkaloids include, but are not limited to, vincristine, vinblastine, vindesine, vinorelbine and the like.

Proteasome inhibitors include VELCADE® (bortezomib), MG132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, ACTIMMUNE® (interferon gamma-1b), or interferon gamma-n1, combinations thereof and the like. Other agents include ALFAFERONE®,(IFN-α), BAM-002 (oxidized glutathione), BEROMUN® (tasonermin), BEXXAR® (tositumomab), CAMPATH® (alemtuzumab), CTLA4 (cytotoxic lymphocyte antigen 4), decarbazine, denileukin, epratuzumab, GRANOCYTE® (lenograstim), lentinan, leukocyte alpha interferon, imiquimod, MDX-010 (anti-CTLA-4), melanoma vaccine, mitumomab, molgramostim, MYLOTARG™ (gemtuzumab ozogamicin), NEUPOGEN® (filgrastim), Onco VAC-CL, OVAREX® (oregovomab), pemtumomab (Y-muHMFG1), PROVENGE® (sipuleucel-T), sargaramostim, sizofilan, teceleukin, THERACYS® (Bacillus Calmette-Guerin), ubenimex, VIRULIZIN® (immunotherapeutic, Lorus Pharmaceuticals), Z-100 (Specific Substance of Maruyama (SSM)), WF-10 (Tetrachlorodecaoxide (TCDO)), PROLEUKIN® (aldesleukin), ZADAXIN® (thymalfasin), ZENAPAX® (daclizumab), ZEVALIN® (90Y-Ibritumomab tiuxetan) and the like.

Biological response modifiers are agents that modify defense mechanisms of living organisms or biological responses, such as survival, growth, or differentiation of tissue cells to direct them to have anti-tumor activity and include include krestin, lentinan, sizofiran, picibanil PF-3512676 (CpG-8954), ubenimex and the like.

Pyrimidine analogs include cytarabine (ara C or Arabinoside C), cytosine arabinoside, doxifluridine, FLUDARA® (fludarabine), 5-FU (5-fluorouracil), floxuridine, GEMZAR® (gemcitabine), TOMUDEX® (ratitrexed), TROXATYL™ (triacetyluridine troxacitabine) and the like.

Purine analogs include LANVIS® (thioguanine) and PURI-NETHOL® (mercaptopurine).

Antimitotic agents include batabulin, epothilone D (KOS-862), N-(2-((4-hydroxyphenyl)amino)pyridin-3-yl)-4-methoxybenzenesulfonamide, ixabepilone (BMS 247550), paclitaxel, TAXOTERE® (docetaxel), PNU100940 (109881), patupilone, XRP-9881 (larotaxel), vinflunine, ZK-EPO (synthetic epothilone) and the like.

Compounds of this invention can also be used as radiosensitizeser that enhance the efficacy of radiotherapy. Examples of radiotherapy include external beam radiotherapy, teletherapy, brachtherapy and sealed, unsealed source radiotherapy and the like.

Additionally, compounds having Formula I, II, III, or IV may be combined with other chemptherapeutic agents such as ABRAXANE™ (ABI-007), ABT-100 (farnesyl transferase inhibitor), ADVEXIN® (Ad5CMV-p53 vaccine), ALTOCOR® or MEVACOR® (lovastatin), AMPLIGEN® (poly I:poly C12U, a synthetic RNA), APTOSYN® (exisulind), AREDIA® (pamidronic acid), arglabin, L-asparaginase, atamestane (1-methyl-3,17-dione-androsta-1,4-diene), AVAGE® (tazarotene), AVE-8062 (combreastatin derivative) BEC2 (mitumomab), cachectin or cachexin (tumor necrosis factor), canvaxin (vaccine), CEAVAC® (cancer vaccine), CELEUK® (celmoleukin), CEPLENE® (histamine dihydrochloride), CERVARIX® (human papillomavirus vaccine), CHOP® (C: CYTOXAN® (cyclophosphamide); H: ADRIAMYCIN® (hydroxydoxorubicin); O: Vincristine (ONCOVIN®); P: prednisone), CYPAT™ (cyproterone acetate), combrestatin A4P, DAB(389)EGF (catalytic and translocation domains of diphtheria toxin fused via a His-Ala linker to human epidermal growth factor) or TransMID-107R™ (diphtheria toxins), dacarbazine, dactinomycin, 5,6-dimethylxanthenone-4-acetic acid (DMXAA), eniluracil, EVIZON™ (squalamine lactate), DIMERICINE® (T4N5 liposome lotion), discodermolide, DX-8951f (exatecan mesylate), enzastaurin, EPO906 (epithilone B), GARDASIL® (quadrivalent human papillomavirus (Types 6, 11, 16, 18) recombinant vaccine), GASTRIMMUNE®, GENASENSE®, GMK (ganglioside conjugate vaccine), GVAX® (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, JUNOVAN™ or MEPACT™ (mifamurtide), lonafamib, 5,10-methylenetetrahydrofolate, miltefosine (hexadecylphosphocholine), NEOVASTAT® (AE-941), NEUTREXIN® (trimetrexate glucuronate), NIPENT® (pentostatin), ONCONASE® (a ribonuclease enzyme), ONCOPHAGE® (melanoma vaccine treatment), ONCOVAX® (IL-2 Vaccine), ORATHECIN™ (rubitecan), OSIDEM® (antibody-based cell drug), OVAREX® MAb (murine monoclonal antibody), paditaxel, PANDIMEX™ (aglycone saponins from ginseng comprising 20(S)protopanaxadiol (aPPD) and 20(S)protopanaxatriol (aPPT)), panitumumab, PANVAC®-VF (investigational cancer vaccine), pegaspargase, PEG Interferon A, phenoxodiol, procarbazine, rebimastat, REMOVAB® (catumaxomab), REVLIMID® (lenalidomide), RSR13 (efaproxiral), SOMATULINE® LA (lanreotide), SORIATANE® (acitretin), staurosporine (Streptomyces staurospores), talabostat (PT100), TARGRETIN® (bexarotene), TAXOPREXIN® (DHA-paclitaxel), TELCYTA® (canfosfamide, TLK286), temilifene, TEMODAR® (temozolomide), tesmilifene, thalidomide, THERATOPE® (STn-KLH), thymitaq (2-amino-3,4-dihydro-6-methyl-4-oxo-5-(4-pyridylthio)quinazoline dihydrochloride), TNFERADE™ (adenovector: DNA carrier containing the gene for tumor necrosis factor-α), TRACLEER® or ZAVESCA® (bosentan), tretinoin (Retin-A), tetrandrine, TRISENOX® (arsenic trioxide), VIRULIZIN®, ukrain (derivative of alkaloids from the greater celandine plant), vitaxin (anti-alphavbeta3 antibody), XCYTRIN® (motexafin gadolinium), XINLAY™ (atrasentan), XYOTAX™ (paclitaxel poliglumex), YONDELIS® (trabectedin), ZD-6126, ZINECARD® (dexrazoxane), ZOMETA® (zolendronic acid), zorubicin and the like.

### Cationic-Based Lipid Encapsulation Systems (CaBLES) and Lipid-Based Particles

CaBLES comprise one or more non-cationic lipids, one or more cationic lipids having Formula I, II, III, or IV and one or more polyethylene glycol (PEG)-lipid conjugate.

Lipid-Based Particles of the present invention are defined as CaBLES which further comprise one or more therapeutic agent(s). These particles have mean diameter sizes of 50-300 nm, of which 50-250 nm is preferred and 50-200 nm is most preferred. Functional CaBLES effectively encapsulate nucleic acids, (e.g., single stranded or double stranded DNA, single stranded or double stranced RNA, RNAi, siRNA, and the like). Suitable nucleic acids include, but are not limited to, plasmids, antisense oligonucleotides, ribozymes as well as other poly- and oligonucleotides. In preferred embodiments, the nucleic acid encodes a product, e.g., a therapeutic product, of interest. The CaBLES of the present invention can be used to deliver the nucleic acid to a cell (e.g., a cell in a mammal) for, e.g., expression of the nucleic acid or for silencing of a target sequence expressed by the cell.

In some embodiments, the nucleic acid is a siRNA molecule that silences the gene of interest, with efficiencies from about 50-100%, and more preferably between about 80-100%.

In other embodiments, the therapeutic agents that can be delivered with CaBLES include RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), chimeric nucleic acids, an antigen, fragments thereof, a protein, a peptide, small-molecules, or mixtures thereof. This invention describes delivery of RNA's such as small inhibitory RNA or microRNA. The siRNA can have varying lengths (10-200 bps) and structures (hairpins, single/double strands, bulges, nicks/gaps, mismatches) and processed in the cell to provide active gene silencing. In certain embodiments of this invention, a double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Suitable siRNA sequences can be identified using means known in the art (e.g., methods described in Elbashir, et al., Nature 411:494-498 (2001) and Elbashir, et al., EMBO J. 20: 6877-6888 (2001) are combined with rational design rules set forth in Reynolds et al., Nature Biotech. 22(3):326-330 (2004)). Further enhancing, isolating, synthesizing and generating of the siRNA can be done by various methods known in the art, (see, e.g., Elbashir, et al., EMBO J. 20: 6877-6888 (2001); Elbashir, et al., Genes Dev. 15:188 (2001); Nykanen, et al., Cell 107:309 (2001)) or may lack overhangs (i.e., to have blunt ends): and Gubler & Hoffman, Gene 25:263-269 (1983); Sambrook et al., Molecular Cloning, A Laboratory Manual (2nd ed. 1989); Current Protocols in Molecular Biology (Ausubel et al., eds., 1994), as are PCR methods (see U.S. Pat. Nos. 4,683,195 and 4,683,202; PCR Protocols: A Guide to Methods and Applications (Innis et al., eds, 1990)).

Non-cationic lipids have a neutral charge or an anionic charge at physiological pH. A neutral lipid, also known as a “helper lipid,” has no net charge at physiological pH. These lipids can also be zwitterionic.

Polyethylene glycol (PEG)-lipid conjugates are used to minimize particle aggregation in solution, provide increased in vivo serum circulation, and enhance distribution of nanoparticles to organs, tissues, cell types, and tumors of interest. These shielding lipids consist of a lipid portion linked to a “PEG” portion via carbamate, ester, amide, ether, amine, thioether, or dithiol linkages. “PEG” is a polyethylene glycol consisting of repeating C₂H₄O units with an average molecular weight between 500 to 10,000 daltons and may be substituted by alkoxy, acyl, alkyl, or aryl. Additionally, the PEG can be substituted at its terminus with one or more of the following functional groups: hydroxy, methoxy, primary, secondary, or tertiary amine, thiol, thioether, thiopyridyl, dithiol, maleimide, or ester.

In some instances it may be desirable for the CaBLES and/or Lipid Based Particles to target using targeting moieties that are specific to a cell type or tissue. Targeting of liposomes using a variety of targeting moieties, such as ligands, cell surface receptors, glycoproteins, vitamins, (e.g., ribolflavin) and moncoleonal antibodies, has been previously described (see, e.g., U.S. Pat. Nos. 4,957,773 and 4,603,044). The targeting moeities can comprise the entire entire protein or fragments thereof. In one aspect, the targeting moiety is a small protein, or peptide. In another aspect, the targeting moiety is a small-molecule.

Cationic lipids are those having one or more moieties that are positively charged at a physiologically relevant pH, typically between 4-8. Particular cationic lipids are as shown in Formula I, II, III, or IV. Examples of cationic lipids that are useful for the practice of this invention include, but are not limited to, N,N-dioleyl-N,N-dimethylammonium chloride, DC-Chol, 1,3-dioleoyloxy-2-(6-carboxyspermyl)-propyl amide, dioctadecylamidoglycyl spermine, N,N-distearyl-N,N-dimethylammonium bromide, N-(2,3-dioleyloxy)propyl)-N,N-dimethylammonium chloride, 1,2-dioleoyl-3-trimethylammonium-propane chloride, 1,2-dilineoyl-3-dimethylammonium-propane, N-(1-(2,3-dioleyloxy)propyl)-N,N,N-trimethylammonium chloride, 1,2-dioleoyl-3-dimethylammonium propane, 1,2-distearyloxy-N,N-dimethyl-3-aminopropane; didodecyldimethylammonium bromide, dioleoyloxy-N-(2-sperminecarboxamido)ethyl)-N,N-dimethyl-1-propanaminiumtrifluoroacetate, 1,2-dimyristyloxypropyl-3-dimethyl-hydroxyethyl ammonium bromide, 1,2-dioleoylcarbamyl-3-dimethylammoniumpropane, tetramethyltetrapalmitoyl spermine, tetramethyltetraoleyl spermine, tetramethyldioleyl spermine, tetramethyltetramyristyl spermine, tetramethyltetralauryl spermine, 1-{3,4-bis[(9Z,12Z)-octadeca-9,12-dienyloxy]butyl}piperidine, 4-{3,4-bis[(9Z,12Z)-octadeca-9,12-dienyloxy]butyl}morpholine N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, 1-{3,4-bis[(9Z,12Z)-octadeca-9,12-dienyloxy]butyl}-4-phenylpiperazine, 1-{3,4-bis[(9Z,12Z)-octadeca-9,12-dienyloxy]butyl}-4-methylpiperazine, N-(2-methoxyethyl)-N-methyl-3,4-bis[(9Z,12Z)-octadeca-9,12-dienyloxy]butan-1-amine, 1-{3,4-bis[(9Z,12Z)-octadeca-9,12-dienyloxy]butyl}-4-(2-methoxyphenyl)piperazine, N-{3,4-bis[(9Z,12Z)-octadeca-9,12-dienyloxy]butyl}-N,N′,N′-trimethylethane-1,2-diamine, N-{3,4-bis[(9Z,12Z)-octadeca-9,12-dienyloxy]butyl}-N-methyl-N-(2-pyridin-2-ylethyl)amine, N-benzyl-N-{3,4-bis[(9Z,12Z)-octadeca-9,12-dienyloxy]butyl}-N-methylamine, N-{3,4-bis[(9Z,12Z)-octadeca-9,12-dienyloxy]butyl}-N-(4-fluorobenzyl)-N-methylamine, 1-{3,4-bis[(9Z,12Z)-octadeca-9,12-dienyloxy]butyl}-4-(2-fluorophenyl)piperazine, N-benzyl-N-{3,4-bis[(9Z,12Z)-octadeca-9,12-dienyloxy]butyl}-N-ethylamine, N-{3,4-bis[(9Z,12Z)-octadeca-9,12-dienyloxy]butyl}-N-ethyl-N′,N′-dimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, N,N-bis(2-methoxyethyl)-3,4-bis[(9Z,12Z)-octadeca-9,12-dienyloxy]butan-1-amine, 1-{3,4-bis[(9Z,12Z)-octadeca-9,12-dienyloxy]butyl}-4-methoxypiperidine, 1-{(3R)-3,4-bis[(9Z,12Z)-octadeca-9,12-dienyloxy]butyl}pyrrolidine, 1-{(3S)-3,4-bis[(9Z,12Z)-octadeca-9,12-dienyloxy]butyl}pyrrolidine, N-{(3R)-3,4-bis[(9Z,12Z)-octadeca-9,12-dienyloxy]butyl}-N,N-diethylamine, N-{(3S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 2-(2-{3,4-bis[(9Z,12Z)-octadeca-9,12-dienyloxy]butoxy}ethyl)-1-methylpyrrolidine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis[(9Z,12Z)-octadeca-9,12-dienyloxy]butoxy}ethyl)-N,N-dimethylamine, 4-(diethylamino)-2-[(9Z,12Z)-octadeca-9,12-dienoyloxy]butyl (9Z,12Z)-octadeca-9,12-dienoate, 1-(2-{3,4-bis[(9Z,12Z)-octadeca-9,12-dienyloxy]butoxy}ethyl)pyrrolidine, N,N-diethyl-N-(2-{2-[(8Z,11Z)-heptadeca-8,11-dienyl]-2-[(9Z,12Z)-octadeca-9,12-dienyl]-1,3-dioxolan-4-yl}ethyl)amine, 1-{[(9Z)-octadec-9-enoyloxy]methyl}-3-pyrrolidin-1-ylpropyl (9Z)-octadec-9-enoate, 1-{3,4-bis[(9Z)-octadec-9-enyloxy]butyl}pyrrolidine, 1-{[(5Z,8Z,11Z,14Z)-icosa-5,8,11,14-tetraenoyloxy]methyl}-3-pyrrolidin-1-ylpropyl (5Z,8Z,11Z,14Z)-icosa-5,8,11,14-tetraenoate, (3S)-3,4-bis[(9Z,12Z)-octadeca-9,12-dienyloxy]butyl 3-pyrrolidin-1-ylpropylcarbamate, 1-[3,4-bis(octadecyloxy)butyl]pyrrolidine, 1-[3,4-bis(hexadecyloxy)butyl]pyrrolidine, 1-{3,4-bis[(9E)-hexadec-9-enyloxy]butyl}pyrrolidine, 1-{3,4-bis[(9E)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N¹-{(3S)-3,4-bis[(9Z,12Z)-octadeca-9,12-dienyloxy]butyl}-N³,N³-diethyl-beta-alaninamide, N-{3,4-bis[(9Z,12Z)-octadeca-9,12-dienyloxy]butyl}-N-[3-(1H-imidazol-1-yl)propyl]amine, N-{3,4-bis[(9Z,12Z)-octadeca-9,12-dienyloxy]butyl}-N,N′,N′-trimethylpropane-1,3-diamine, 1-(1-{3,4-bis[(9Z,12Z)-octadeca-9,12-dienyloxy]butyl}pyrrolidin-3-yl)-1H-imidazole, N-{3,4-bis[(9Z,12Z)-octadeca-9,12-dienyloxy]butyl}-N-(3-pyrrolidin-1-ylpropyl)amine, N-{3,4-bis[(9Z,12Z)-octadeca-9,12-dienyloxy]butyl}-N′,N′-dimethylpropane-1,3-diamine, 1-{3,4-bis[(9Z,12Z)-octadeca-9,12-dienyloxy]butyl}azetidine, 1-{3,4-bis[(9Z,12Z)-octadeca-9,12-dienyloxy]butyl}-2-methylpyrrolidine, and 1-{3,4-bis[(9Z,12Z)-octadeca-9,12-dienyloxy]butyl}-2,5-dimethylpyrrolidine, and mixtures thereof.

Lipid-Based Particles are a mixture of one or more cationic lipids of Formula I, II, III, or IV (I), one or more non-cationic lipids, one or more PEG-lipid conjugates, and one or more therapeutic agents. Specific Lipid-Based Particles comprise the following lipid mixtures: cationic lipid(s) (about 2-60% by weight), non-cationic lipid(s) (about 5-90% by weight), and PEG-lipid conjugate(s) (about 0.1-20%).

### Tables 1 and 2

### Representative In-vitro Formulation of Lipid-Based Particles

TABLE 1

| Therapeutic | | |
|---|---|---|
| Agent | Mass (mg) | Vol (μL in water 10 mg/mL) |
| siSTABLE | 0.20 | 20 |
| | Mass (mg) | Vol (μL in ethanol 10 mg/mL) |
| Total Lipids | 5.0 | 500 |

TABLE 2

| | Wt % | Mass (mg) | Vol (μL in ethanol, 10 mg/mL) |
|---|---|---|---|
| Non-cationic | 10 | 0.5 | |
| lipid | | | |
| PEG-lipid | 15 | 0.75 | |
| Cholesterol | 30 | 1.5 | |
| | | Total Volume | 275 |
| Cationic lipid | 45 | 2.25 | 225 |

### Preparation of Lipid Mixture Solution

The mixing solution of cationic lipids, cholesterol, non-cationic lipids and PEG-lipids was prepared in ethanol (total concentration at 10 mg/mL). siSTABLE (purchased from ThermoFisher) (sense-5′ GGG GAA AGC UGG CAA GAU UUU-3′SEQ ID NO. 1, antisense-5′-AAU CUU GCC AGC UUU CCC CUU-3′ SEQ ID NO: 2) % stock solution was prepared in 10 mg/mL of solution by dissolving 10 mg siRNA in 1 mL of RNAse-free UltraPure Water. The calculated amount of siRNA solution was added to 1 mL of citrate buffer (pH 4.0, 20 mM), to provide an siRNA concentration of 0.2 mg/mL, and warmed to 60 C. The calculated amount of lipid solution was warmed to 60 C, transferred to a 0.5 mL syringe with 28½ gauge needle, and injected into the citrate buffer with stirring at 60 C. After 3 minutes, 3 mL of PBS solution at room temperature (pH 7.4) was added into the lipid mixture with stirring. The Lipid-Based Particle solution was cooled to room temperature.

### Analysis of Lipid-Based Particles

The siRNA concentrations were measured using Quanti-iT RiboGreen RNA reagent (Molecular Probes, (R11490)). Vesicle sizes were characterized by dynamic light scattering with a DynaPro™ Plate Reader (Wyatt Technology) in 96-well half-area UV plate (Coring) after diluting the formulation sample (20 μL) in phosphate buffered saline (80 μL) at a pH of about 7-8. A 1% agarose gel-based assay was used for analyzing nuclease degradation and protection. Encapsulation efficiency (EE) was calculated using data obtained from a RiboGreen assay.

### Ribogreen Assay for Measuring SiRNA Concentration and Encapsulation Efficiency of of Lipid-Based Particles

RNA concentration and encapsulation efficiency were determined using a Quant-iT® Ribogreen RNA reagent and kit available from Invitrogen. The siRNA was released from the Lipid-Based Particle using one of the following reagents: ethanol, Triton X-100, or phenol/chloroform. The siRNA concentration is quantified using fluorescent reading at 480 nm/520 nm.

### Particle Sizing Assay

Particle sizes and size distributions (PDI) were characterized by using dynamic light scattering (DLS). A DLS plate reader (Dynapro™, Wyatt Technology) was used for the DLS measurement. This DLS plate reader uses an 830 nm laser and the scattering angle is 158°. It also can control temperature from 4° C. to 70° C. A 96-well format was employed for the samples.

Samples for DLS analysis were prepared by mixing 20 μL of each sample stock solution with 80 μL PBS directly in the 96-well plate (#3697, Corning). Sample mixing was accomplished using a microplate shaker (Orbis, Mikura Ltd.). Plates were read at 20° C. with an acquisition time of 50 seconds for each sample, and data was analyzed with Wyatt Technology's Dynamics V6 software. To rule out potential multiple scattering artifacts, a second plate at 4-fold reduced sample concentrations was independently prepared by mixing 5 μL stock solutions with 95 μL PBS. Under our experimental conditions the results at the two concentrations were very similar, and the final reported result for each sample represents the average of values obtained from the two plates.

TABLE 3
In vitro Formulation Data Table of Particle Size, Encapsulation
Efficiency, and Size Distributions

| | | % | |
|---|---|---|---|
| | | Encapsulation | Size (PDI) |
| Example | Formulation | Efficiency | (nm) |
| 1 | b | nd | 96.3 |
| 3 | a | 97.2 | 192 |
| 3 | b | 96.9 | 113.4 |
| 4 | b | 97.5 | 136.6 |
| 5 | a | nd | 157 (0.070) |
| 6 | a | nd | 178 (0.16) |
| 7 | b | 96.9 | 123.2 |
| 8 | a | nd | 130 |
| 9 | b | 88.3 | 105.8 |
| 10 | a | nd | 161 (0.168) |
| 11 | a | nd | 154 (0.2) |
| 12 | a | nd | nd |
| 13 | a | nd | nd |
| 14 | a | nd | nd |
| 15 | nd | nd | nd |
| 16 | a | nd | 70.9 |
| 17 | b | 86.2 | 114.4 |
| 18 | a | 94.1 | 104 |
| 19 | a | 94.2 | 152 |
| 20 | a | 97.2 | 146 |
| 21 | a | 96.9 | 128 |
| 22 | a | 86 | 130 |
| 23 | a | 97.2 | 128 |
| 24 | b | 97.0 | 135.4 |
| 25 | b | 96.1 | nd |
| 26 | b | 99.9 | nd |
| 27 | b | 97 | nd |
| 28 | b | 94.3 | nd |
| 29 | b | 96.4 | nd |
| 30 | a | 96.8 | 134 |
| 31 | b | 96.2 | nd |
| 32 | b | 98.6 | nd |
| 33 | b | 99 | nd |
| 34 | b | 99 | nd |
| 35 | b | 83.5 | nd |
| 36 | b | 99 | nd |
| 37 | b | 98.5 | nd |
| 38 | b | 99 | nd |
| 39 | b | 99 | nd |
| 40 | b | 97 | nd |
| 41 | b | 99 | nd |
| 42 | b | 98 | nd |
| 43 | b | 96.5 | nd |
| 44 | b | 98 | nd |
| 45 | b | 99 | nd |
| 46 | b | 98 | nd |
| 47 | b | 99 | nd |
| 48 | b | 99 | nd |
| 49 | b | 99 | nd |
| 50 | b | 99 | nd |
| 51 | b | 99 | nd |

nd = not determined

TABLE 4
In vitro and In vivo Formulations

| Formulation | | Lipid Ratio | Lipid:siRNA |
|---|---|---|---|
| Designation | Composition | (wt %) | ratio |
| A/a | Cationic lipid/Example 73/DSPC/Chol | 45/10/15/30 | 25:1 |
| B/b | Cationic lipid/PEG- | 45/10/15/30 | 25:1 |
| | cholesterol/DSPC/Chol | | |
| C | Cationic lipid/Pal-PEG-Cera/DSPC/Chol | 45/10/15/30 | 25:1 |
| D | Cationic lipid/Example 66/DSPC/Chol | 45/10/15/30 | 25:1 |
| E | Cationic lipid/Example 53/DSPC/Chol | 45/10/15/30 | 25:1 |
| F | Cationic lipid/Example 54/DSPC/Chol | 45/10/15/30 | 25:1 |
| G | Cationic lipid/Example 55/DSPC/Chol | 45/10/15/30 | 25:1 |
| H | Cationic lipid/PEG-DMPE/DSPC/Chol | 45/10/15/30 | 25:1 |
| I | Cationic lipid/PEG-DPPE/DSPC/Chol | 45/10/15/30 | 25:1 |
| J | Cationic lipid/PEG-DSPE/DSPC/Chol | 45/10/15/30 | 25:1 |
| K | Cationic lipid/PEG-DMG/DSPC/Chol | 45/10/15/30 | 25:1 |
| L | Cationic lipid/PEG-DPG/DSPC/Chol | 45/10/15/30 | 25:1 |
| M | Cationic lipid/PEG-DSG/DSPC/Chol | 45/10/15/30 | 25:1 |
| N | Cationic lipid/PEG-DPG/DOPE/Chol | 45/10/15/30 | 25:1 |
| MM | Cationic lipid/PEG-DPG/DOPC/Chol | 45/10/15/30 | 25:1 |
| O | Cationic lipid/PEG-DPG/SPC/Chol | 45/10/15/30 | 25:1 |
| P | Cationic lipid/PEG-DSPE/DOPE/Chol | 45/10/15/30 | 25:1 |
| Q | Cationic lipid/PEG-DSPE/DOPC/Chol | 45/10/15/30 | 25:1 |
| R | Cationic lipid/PEG-DSPE/SPC/Chol | 45/10/15/30 | 25:1 |
| S | Cationic lipid/PEG-DMG/PEG-DPG/DSPC/Chol | 44/4.5/4.5/14/33 | 25:1 |
| T | Cationic lipid/PEG-DMG/PEG-DSG/DSPC/Chol | 44/4.5/4.5/14/33 | 25:1 |
| U | Cationic lipid/PEG-DPG/PEG-DSG/DSPC/Chol | 44/4.5/4.5/14/33 | 25:1 |
| V | Cationic lipid/PEG-DMPE/PEG-DPPE/DSPC/Chol | 44/4.5/4.5/14/33 | 25:1 |
| W | Cationic lipid/PEG-DPPE/PEG-DSPE/DSPC/Chol | 44/4.5/4.5/14/33 | 25:1 |
| X | Cationic lipid/PEG-DMPE/PEG-DSPE/DSPC/Chol | 44/4.5/4.5/14/33 | 25:1 |
| Y | Cationic lipid/Example 54/PEG-DSG/DSPC/Chol | 44/4.5/4.5/14/33 | 25:1 |
| Z | Cationic lipid/PEG-DMG/PEG-DSPE/DSPC/Chol | 44/4.5/4.5/14/33 | 25:1 |
| AA | Cationic lipid/PEG-DSG/PEG-DMPE/DSPC/Chol | 44/4.5/4.5/14/33 | 25:1 |
| BB | Cationic lipid/Example 54/PEG-DSPE/DSPC/Chol | 44/4.5/4.5/14/33 | 25:1 |
| CC | Cationic lipid/Example 54/DSPC/Chol | 46/5/15/34 | 25:1 |
| DD | Cationic lipid/Example 54/DSPC/Chol | 44/9/14/33 | 25:1 |
| EE | Cationic lipid/Example 54/DSPC/Chol | 42/13/13/32 | 25:1 |
| FF | Cationic lipid/PEG-DSPE/DSPC/Chol | 46/5/15/34 | 25:1 |
| GG | Cationic lipid/PEG-DSPE/DSPC/Chol | 45/7/14/34 | 25:1 |
| HH | Cationic lipid/PEG-DPPE/PEG- | 44/4.5/4.5/14/33 | 15:1 |
| | DSPE/DSPC/Chol | | |
| II | Cationic lipid/PEG-DPPE/PEG- | 44/4.5/4.5/14/33 | 10:1 |
| | DSPE/DSPC/Chol | | |
| JJ | Cationic lipid/Example 54/DSPC/chol | 44/9/33/14 | 15:1 |
| KK | Cationic lipid/Example 54/DSPC/chol | 44/9/33/14 | 10:1 |
| LL | Cationic lipid/PEG-DMPE/PEG- | 44/4.5/4.5/14/33 | 25:1 |
| | DPG/DSPC/Chol | | |
| NN | Cationic lipid/Example 74/DSPC/Chol | 45/10/15/30 | 25:1 |
| OO | Cationic lipid/Example 75/DSPC/Chol | 45/10/15/30 | 25:1 |
| PP | Cationic lipid/Example 73/Chol | 45/10/45 | 25:1 |

TABLE 5
Representative In vivo Formulation of Lipid-Based Particles

| | Cationic | | | | |
|---|---|---|---|---|---|
| | lipid | PEG-lipid | Non-cationic lipid | Cholesterol | Total |
| % (w/w) | 44 | 9 | 14 | 33 | 100 |
| Weight | 54.9 | 11.2 | 17.6 | 41.4 | |
| (mg) | | | | | |
| Volume | 5.49 | 1.12 | 1.76 | 4.14 | |
| (ml) | | | | | |

### Preparation of Lipid Mixture Solution

The lipid solution was prepared (10 mg/ml) by dissolving the lipid in 200 proof ethanol. The lipid mixture solution is prepared according to the above composition in Table 5.

### Preparation of siRNA Solution

An siRNA (TetR_ODC_{—}12, G.G.G.G.A.A.A.G.C.U.G.G.C.A.A.G.A.U.U.U.U SEQ ID NO: 1) (ThermoFisher) solution is prepared in a concentration of 10 mg/ml by dissolving 10 mg siRNA in 1 ml of DNAse/RNAse-free distilled water.

### Preparation of Lipid-Based Particles

A round bottom flask was submerged into a 65° C. water bath. Citrate buffer (37.5 ml) of pH 4.0 was pipetted into the flask. The solution was stirred by a magnetic stirring bar at a speed of 900 rpm. Both the pH 4.0 citrate buffer and the lipid solution were pre-warmed in the 65° C. water bath for about 3 minutes. A siRNA solution (0.5 ml) was pipetted into the pH 4.0 citrate buffer. The 12.5 ml lipid mixture solution was injected through a 27 gauge needle into the citrate buffer in about 30 seconds. The needle tip was inserted into the solution during the injection. The resulting solution was stirred for 5 minutes at a speed of 900 rpm. The flask was pulled up from the water bath and a 50 ml pH 7.4 PBS buffer was added into the flask. The final solution was further mixed at a speed of 900 rpm for 5 minutes. For the diafiltration process, a dialysis filter (Millipore, 100K, Cat#PXB100C50) was used to remove ethanol in the above solution. When the volume was reduced to 20 ml during the initial diafiltration, 20 ml of pH 7.4 PBS was added to the sample solution. The diafiltration was continued until the volume was reduced to 20 ml. The diafiltration process was repeated 4 times. The volume of the sample solution was reduced to about 12 ml and pH 7.4 PBS was added to make the final volume of 15 ml. The 15 ml solution was filtered sequentially through the 0.45 and 0.22 μm sterile PVDF membrane filters (Millipore) and immediately transferred into a sterile vial.

### Particle Sizing Assay

For measurements of particle sizes and size distributions (PDI), lipid-based particles were prepared as described above. The particle solution (60 μL) was pipetted into a disposable cuvette (UVette, Eppendorf, cat#952010051) and measured in the “General Purpose” mode. Attenuator and position were optimized by the device. Measurements were performed using a Zetasizer Nano ZS (Malvern Instruments) equipped with a 4 mW He—Ne laser at a wavelength of 633 nm at 25° C. Scattered light was detected at a 173° backward scattering angle. The viscosity and refractive index of water at 25° C. was used for data analysis with the Dispersion Technology Software 5.00 (Malvern Instruments).

TABLE 6
In vivo Formulation Data Table of Particle Size, Encapsulation
Efficiency, and Size Distributions

| | | % | | |
|---|---|---|---|---|
| | | Encapsulation | Size (PDI) | |
| Example | Formulation | Efficiency | (nm) | |
| 1 | A | 74 | 108 | (0.18) |
| 2 | A | 83.6 | 89 | (0.1) |
| 3 | A | nd | 116 | (0.12) |
| 4 | A | nd | 118, 479 | (81:19%) |
| 7 | A | 74 | 140 | (0.15) |
| 9 | A | 87.5 | 160 | (0.19) |
| 9 | I | 98 | 93.4 | (0.069) |
| 9 | J | 98 | 98.1 | (0.078) |
| 9 | L | 98 | 98.7 | (0.056) |
| 17 | A | nd | 149 | (0.21) |
| 17 | C | 97 | 101 | (0.154) |
| 20 | A | nd | 100 | (0.09) |
| 21 | A | nd | 103 | (0.06) |
| 24 | D | 97.0 | 164 | (0.104) |
| 24 | E | 79.3 | 169 | (0.163) |
| 24 | F | 96.1 | 152 | (0.08) |
| 24 | G | 96.7 | 164 | (0.104) |
| 24 | H | 97.3 | 168 | (0.007) |
| 24 | I | 92 | nd | |
| 24 | J | 100 | 119.7 | (0.006) |
| 24 | K | 99.5 | 155 | (0.052) |
| 24 | L | 90 | nd | |
| 24 | M | 100 | 116.5 | (0.029) |
| 24 | P | 97 | 153.7 | (0.061) |
| 24 | Q | 97 | 144.1 | (0.01) |
| 24 | R | 97 | 159.6 | (0.036) |
| 28 | I | 99 | 107.1 | (0.025) |
| 28 | J | 99 | 80.2 | (0.026) |
| 28 | L | 99 | 87 | (0.012) |
| 31 | A | 93.3 | 136 | (0.0923) |
| 24 | A | 96.6 | 96 | (0.06) |
| 24 | B | 89.3 | 327 | (0.198) |
| 24 | C | 97.8 | 141 | (0.026) |
| 24 | NN | 97.3 | 140 | (0.029) |
| 24 | OO | 99.5 | 142 | (0.029) |
| 24 | PP | 97.4 | 108.7 | (0.094) |

nd = not determined

### Determination of Transfection Efficiency of MDA435-TetR-Luc Cells with Lipid-Based Particles

To determine the knockdown efficacy of Lipid-Based Particles in an in vitro assay, MDA435-TetR-Luc cells (The positive readout reporter cell line MDA435-TetR-Luc contained a stably integrated copy of the luciferase gene expressed from a CMV promoter containing the tetR operator site. In addition, gene coding for a destabilized TetR protein was expressed in this cell line.) were plated in 96 well plate at a density of 10K cells per well in 100 ul of DMEM (Dulbecco's Modified Eagles Medium, Invitrogen Corp.) containing 10% fetal bovine serum (Invitrogen Corp.). Appropriate dilutions of Lipid-Based Particles were made in DMEM+10% fetal bovine serum medium, 10 ul of the diluted material was transferred into each well in triplicate. Transfected cells were further incubated at 37° C. for a period of 72 hours. Supernatent from each well was removed and cells were assayed for luciferase activity (Steady Glo kit, ProMega Corp.) as per the manufacturers recommendation. Positive controls included cells treated with 100 ul of doxycycline at 0.5 mg/ml, 20 nM tetR siRNA transfected with lipofectamine (Invitrogen Corp.) or untreated cells. The graphs represent average of triplicate readings of the Lipid-Based Particles treated sample divided by the average of readings from 9 wells treated with doxycycline.

### Tumor Models

The animal studies were carried out in accordance with internal Institutional Animal Care and Use Committee (IACUC) guidelines at Abbott Laboratories. Scid female mice at 6 to 8 weeks of age were obtained from Charles River Laboratory and used for intraliver tumor models. Mouse livers were exposed by vertical incision on mouse abdomens and the tumor cells were directly injected into the livers. The incision was closed by suture and wound clips. All cell lines used for creating xenograft tumors were subjected to the IMPACT profile I test (18 agents) at the University of Missouri Research Animal Diagnostic and Investigative Laboratory, and all cell lines were found negative for the 18 infectious agents tested. Tumor cells were suspended in a 1:1 mixture of S-MEM (Invitrogen, Carlsbad, Calif.) and matrigel (BD Bioscience, San Jose, Calif.) and inoculated at 1×10E6 cells per animal.

### Animal Dosing and Sample Harvesting

Treatments were started 3˜4 weeks after tumor inoculation. Formulated or unformulated siRNAs were administrated via tail vein (i.v) injection.

### IHC Analysis

IHC was carried out as previously described [Li, L., et al., Evaluating hypoxia-inducible factor-1alpha as a cancer therapeutic target via inducible RNA interference in vivo. Cancer Res, 2005. 65(16): p. 7249-58]. Briefly, tumors were excised, cut into pieces of less than 3 mm in thickness and immediately fixed in buffered formalin solution with neutral pH (Sigma, St. Louis, Mo.). The formalin-fixed and paraffin-embedded tumor sections were then used for staining. The mouse anti-β-galactosidase mAb (Promega, Madison, Wis.) was used to detect β-galactosidase in tumor sections. DAB (3,3′-diaminobenzidine) was used as the chromogen. IHC images were acquired using the Nikon TE2000 inverted microscope. The β-galactosidase staining was evaluated by 2 people independently based on the scoring system listed below. The average of the score was calculated for each tumor.

### IHC Scoring System

−, there was no staining, a small area of weak staining, or disperse strong single cell staining
+/−, there was 5% of the section with weak staining or one patch of strong staining
+, there was 15% of the section with strong staining
++, there was ˜50% of the section with strong straining
+++, there was 80% of the section with strong straining
++++, the whole section had strong staining

TABLE 7
In vivo Response of a Lipid-Based Particle (1-A, 2-A, 3-A, 9-A) versus
a Positive Control (Doxycycline)

| | 1 | 2 | 3 | 4 | 5 | 6 |
|---|---|---|---|---|---|---|
| Doxycycline | +++ | +++ | +++ | +++ | +++ | +++ |
| 1-A | + | −/+ | −/+ | −/+ | −/+ | −/+ |
| 2-A | + | + | −/+ | −/+ | − | − |
| 3-A | + | −/+ | −/+ | −/+ | −/+ | − |
| 9-A | − | − | − | − | − | − |

### Bioluminescence Imaging and Analysis

In vivo bioluminescence imaging and analysis were conducted on the IVIS 200 system using the Living Image acquisition and analysis software (Caliper Life Science, Hopkinton, Mass.). After intra-peritoneal injection of luciferin (Promege, Madison, Wis.) at 150 mg/kg, mice were anesthetized with isofluorane. Four minutes after the injection of luciferin, a series of time-lapse images were acquired at 2 minutes intervals in a total of 10 minutes. Regions of interest (ROI) were drawn around the tumors and signal intensity was quantified as the sum of photon counts per second within the ROI after the subtraction of background luminescence. The peak reading during the 10-minute imaging period was used for calculating the signal ratio before and after siRNA delivery.

The ability of novel cationic lipids to transfect siRNA in vitro was evaluated in the TetRLuc assay. By utilizing a releasable PEG lipid as for examples in formulations “a” or “b”, the transfection efficiency of the unshielded or partially shielded particle may be determined.

### Targeting Lipid-Based Particles

The formulation of a prototypical example of a targeted Lipid-Based Particle began by preparing a stock solution of lipid in absolute ethanol at a concentration of 10 mg/mL. The lipid mixture solution was prepared according to the following composition:

TABLE 8

| | Example | | | | |
|---|---|---|---|---|---|
| | 24 | PEG-lipid^{a} | DSPC | Cholesterol | Total |
| Weight | 54.9 | 8.75 | 17.6 | 41.4 | |
| (mg) | | | | | |
| Volume | 5.49 | 0.875 | 1.76 | 4.14 | 12.5 mL |
| (mL) | | | | | (w/ 0.245 mL |
| | | | | | ethanol) |

^{a}PEG-lipid consisted of a mixture of PEG-DSPE and PEG-DSPE-PDP (1,2-distearoyl-sn-glycero-3-phosphoethanolamine-N-[PDP(polyethylene glycol)-2000], Avanti Polar Lipids).

A round bottom flask was submerged into a 61° C. water bath. A solution of 37.5 mL of pH 4.0 citrate buffer was pipetted into the flask. The solution was stirred by a magnetic bar at a speed of 900 rpm. Both the pH 4.0 citrate buffer and the lipid solution were pre-warmed in the water bath for 10 minutes. A solution of 0.5 mL of siRNA solution (consisting of 5 mg TetR_ODC-12, which had the following sequence: G.G.G.G.A.A.A.G.C.U.G.G.C.A.A.G.A.U.U.U.U SEQ ID NO: 1) or a non-targeted composition, was pipetted into the pH 4.0 citrate buffer. The 12.5 mL lipid mixture solution was injected through a 27 G needle into the citrate buffer in about 30 seconds. The needle tip was inserted into the solution during the injection. The resulting solution was mixed for 5 minutes at a speed of 900 rpm. The flask was removed from the water bath and 50 mL of phosphate buffered saline (PBS, pH 7.4) was added into the flask. The final solution was further mixed at a speed of 900 rpm for 5 minutes.

A dialysis filter (Millipore, 100K, Cat# PXB100C50) was used to remove ethanol in the above solution. When the volume was reduced to 20 mL during the initial filtration, 20 mL of pH 7.4 PBS was added to the sample solution. The filtration was continued until the volume was reduced to approximately 20 mL. This filtration process was repeated another 4 times. The volume of the final sample solution was reduced to 10 mL.

The 10 ml solution was filtered through 0.45 and 0.22 um sterile PVDF membrane filters and immediately transferred into a sterile vial.

### General Procedure for the Synthesis of Targeted Lipid-Based Particles

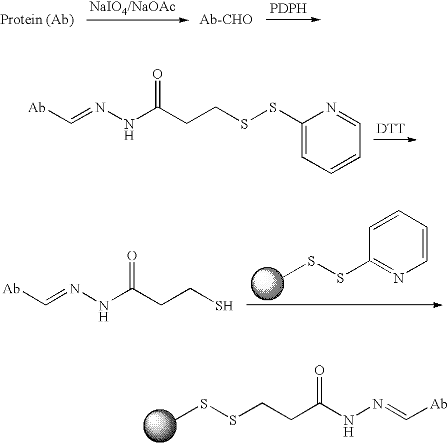

The carbohydrate groups of the targeting glycoprotein, in the case when the putative targeting ligands are antibodies, such as the known anti-human CD71 clone OKT-9 (eBioscience Cat#14-0719), were oxidized using sodium periodate (NaIO₄). A solution of 20 mM sodium periodate was prepared in 100 mM sodium acetate (NaIO₄), pH 5.5. An equal volume of this solution (in the ratio of 1 mL of sodium periodate per 1 mL glycoprotein) was added to the protein to give a final concentration of 10 mM sodium periodate. The protein concentration was typically around 10 to 30 mg/mL in 100 mM sodium acetate at pH 5.5. The protein/sodium periodate solution was then mixed at 4° C. for 30 minutes, protected from light. After mixing, excess periodate was quenched by the addition of 1,2-propanediol. The oxidized protein was purified by spin filtration (Amicon Ultra, MWCO=50,000) followed by size exclusion chromatography (Bio-Rad 10DG columns, MWCO=6,000) using 100 mM sodium phosphate, 150 mM NaCl, pH 7.2. The concentration of the protein solution was determined by UV-Visible spectroscopy at 280 nm (extinction coefficient at 280 nm of the proteins typically varies from 1 to 2×10⁵ M⁻¹cm⁻¹).

The oxidized protein was then mixed with an excess of PDPH (3-[2-pyridyldithio]propionyl hydrazide, Thermo-Fisher Cat#22301). PDPH was dissolved in a small amount of DMSO (typically 10 mg per 50 uL DMSO) and was added to the protein solution to give a final 10 mM solution, resulting in an approximate 50 fold molar excess of crosslinker to protein. The solution was then incubated at room temperature for approximately 18 hours. Purification was performed using spin filtration followed by size exclusion chromatography. The protein concentration is determined as before (A₂₈₀). The amount of crosslinker covalently bound to the protein is determined by cleaving the pyridyldithiol of the crosslinker using an excess of DTT (dithiothreitol) and measuring the UV-Visible absorbance of the released pyridine-2-thione at 343 nm.

In order to easily quantitate the amount of protein coupled to the liposome, an amine reactive dye, Alexa Fluor 647 succinimidyl ester (Invitrogen A20006), was reacted with the crosslinked protein. In general, about 1 to 2 equivalents of dye (dissolved in DMSO) were mixed with the crosslinked labeled protein. The reaction was incubated at room temperature for 2-3 hours, protected from light. The PDPH-protein-Alexa 647 was purified via spin filtration (MWCO=50,000) followed by gel filtration (MWCO=6,000) using 1×PBS, pH 7.2 (Gibco Cat#20012). The ratio of dye to protein was determined by the UV-Visible absorption of protein at 280 nm and the absorption of the dye at 654 nm (dye correction factor at 280 nm=0.03).

Endotoxin, that is potentially present in the PDPH-protein-Alexa 647 sample, was removed using a 1 mL endotoxin removing column (Detoxi-Gel Endotoxin Removing Gel, Thermo-Scientific Cat#20344). Typically, 2 to 3 mL of modified protein (˜60 mg of protein) was flowed through the column using 1×PBS, pH 7.2. The PDPH-protein-Alexa 647 was then concentrated to approximately 1 mL using a spin filter (MWCO=50,000). The endotoxin level of the final modified protein was determined using the Limulus Amebocyte Lysate assay (Lonza Cat#50-647U).

Next, the PDPH-protein-Alexa 647 was reduced with dithiothreitol (DTT). In general, 2 to 5 equivalents of DTT were added to the protein solution at room temperature. The reaction was monitored by the UV-Vis abosorption of the released pyridine-2-thione at 343 nm. The excess DTT was removed through gel filtration (Bio-Rad 10DG column, MWCO=6,000) using 100 mM sodium phosphate, 150 mM NaCl, 0.2 mM EDTA, pH 7.2 as the column buffer. Concentration of the eluted protein was determined by absorption at 280 nm and 654 nm (dye correction factor at 280 nm=0.03).

Pyridyldithiol loading of the liposomes ranged from 2.5 to 0.2%, while total DSPE-PEG2000 ranged from 10 to 7%. Particles were concentrated about 5 fold using dialysis tubing (MWCO=3000) and Sephadex G-75 to remove water. The actual pyridyldithiol content of the particles was determined by treatment of a small portion with DTT and measuring pyridine-2-thione release (A₃₄₃). A 4-5 fold excess of free thiol on the modified protein was added to the particles and the mixture was incubated at 37° C. for approximately 24 hours. The protein conjugated Lipid-Based particles were then purified using a Sepharose CL-4B column (Sigma Cat# CL4B200, fractionation range 60,000 to 20,000,000—globular proteins, buffer: 1×PBS, pH 7.2). The level of protein labeling was determined by the Alexa 647 absorption at 650 nm. Final endotoxin level of targeted Lipid-Based Particles was determined using the Limulus Amebocyte Lysate assay (Lonza Cat#50-647U).

TABLE 9

| | | | | | siRNA |
|---|---|---|---|---|---|
| | | | | OKT9 | sequence |
| | | Lipid Ratio | | Loading | (TetR or |
| Formulation | Composition | (wt %) | Size (nm) | (μM) | NTC) |
| 1 | Example 24/DSPC/Chol/PEG- | 44/14/33/6.8/0.2 | 121 | .31 | NTC |
| | DSPE/PEG-DSPE-PDP | | | | |
| 2 | Example 24/DSPC/Chol/PEG- | 44/14/33/6.8/0.2 | 116 | .32 | TetR |
| | DSPE/PEG-DSPE-PDP | | | | |
| 3 | Example 24/DSPC/Chol/PEG- | 44/14/33/7 | 123 | 0 | TetR |
| | DSPE | | | | |
| 4 | Example 24/DSPC/Chol/PEG- | 44/14/33/8.8/0.2 | 96 | .29 | TetR |
| | DSPE/PEG-DSPE-PDP | | | | |
| 5 | Example 24/DSPC/Chol/PEG- | 44/14/33/9 | 94 | 0 | TetR |
| | DSPE | | | | |

Without intending on being held to any particular theory, the in vitro transfection efficiency of a given formulation, including the cationic lipids of the present invention, may or may not predict for in vivo delivery. The in vivo delivery may depend upon the properties of other co-lipid components in the formulation. Properties of the co-lipids that may modulate in vivo delivery, include for example, PEG lipid alkyl length, PEG polymer length, concentration of the PEG lipid conjugate, presence and concentration of neutral helper lipid, as well as the manner of which the co-lipid components are formulated (Sadzuka, et. al., J. Liposome Research, 13,2, (2003) 157-172; Sadzuka, et. al., Int. J. Pharm.,312, (2006) 83-89; Li, et. al, Biochimica et Biophysica Acta 1513 (2001) 193-206; Chiu, et al., Biochimica et Biophysica Acta 1560 (2002) 37-50; and Mukherjee, et al., FEBS Letters 579 (2005) 1291-1300.)

The aggregate effect of these co-lipids and their formulation impacts a set of parameters that includes for example particle stabilization, serum stability, circulation half-life, particle internalization, intracellular release of the therapeutic agent. These factors in total are likely to mitigate effective in vivo delivery.

### Synthesis

The following abbreviations have the meanings indicated: ADDP means 1,1′-(azodicarbonyl)dipiperidine; AD-mix-β means a mixture of (DHQD)₂PHAL, K₃Fe(CN)₆, K₂CO₃ and K₂SO₄); AIBN means 2,2′-azobis(2-methylpropionitrile); 9-BBN means 9-borabicyclo(3.3.1)nonane; Cp means cyclopentadiene; (DHQD)₂PHAL means hydroquinidine 1,4-phthalazinediyl diethyl ether; DBU means 1,8-diazabicyclo(5.4.0)undec-7-ene; DCC means dicyclohexylcarbodiimide; DIBAL means diisobutylaluminum hydride; DIEA means diisopropylethylamine; DMAP means N,N-dimethylaminopyridine; DME means 1,2-dimethoxyethane; DMF means N,N-dimethylformamide; dmpe means 1,2-bis(dimethylphosphino)ethane; DMSO means dimethylsulfoxide; dppa means diphenylphosphoryl azide; dppb means 1,4-bis(diphenylphosphino)butane; dppe means 1,2-bis(diphenylphosphino)ethane; dppf means 1,1′-bis(diphenylphosphino)ferrocene; dppm means 1,1-bis(diphenylphosphino)methane; EDAC means 1-(3-dimethylaminopropyl)-3-ethylcarbodiimide; Fmoc means fluorenylmethoxycarbonyl; HATU means O-(7-azabenzotriazol-1-yl)-N,N′N′N′-tetramethyluronium hexafluorophosphate; HMPA means hexamethylphosphoramide; IPA means isopropyl alcohol; LDA means lithium diisopropylamide; LHMDS means lithium bis(hexamethyldisilylamide); MP-BH₃ means macroporus triethylammonium methylpolystyrene cyanoborohydride; LAH means lithium aluminum hydride; NCS means N-chlorosuccinimide; PyBOP means benzotriazol-1-yloxytripyrrolidinophosphonium hexafluorophosphate; TDA-1 means tris(2-(2-methoxyethoxy)ethyl)amine; TEA means triethylamine; TFA means trifluoroacetic acid; THF means tetrahydrofuran; NCS means N-chlorosuccinimide; NMM means N-methylmorpholine; NMP means N-methylpyrrolidine; PPh₃ means triphenylphosphine.

The following schemes are presented to provide what is believed to be the most useful and readily understood description of procedures and conceptual aspects of this invention. Compounds of this invention may be made by synthetic chemical processes, examples of which are shown herein. It is meant to be understood that the order of the steps in the processes may be varied, that reagents, solvents and reaction conditions may be substituted for those specifically mentioned, and that vulnerable moieties may be protected and deprotected, as necessary.

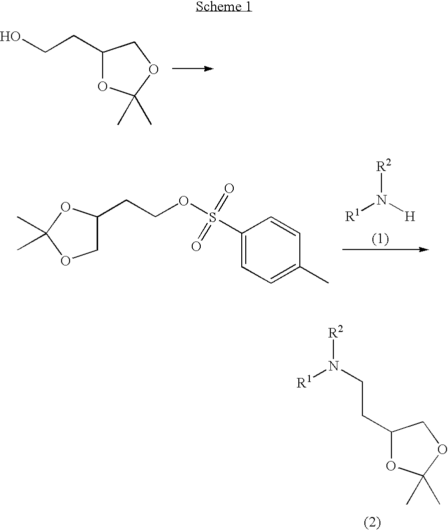

As shown in Scheme 1, 2-(2,2-dimethyl-1,3-dioxolan-4-yl)ethyl 4-methylbenzenesulfonate, which can be prepared as described in Example 1A, when reacted with an amine of Formula (1) wherein R¹ and R2 are as described herein, with a base such as but not limited to N,N-diisopropylethylamine, will provide a compound of Formula (2). The reaction may be conducted in a commercial single mode microwave at elevated temperature. Anhydrous solvents such as but not limited to 1,4-dioxane are typically employed.

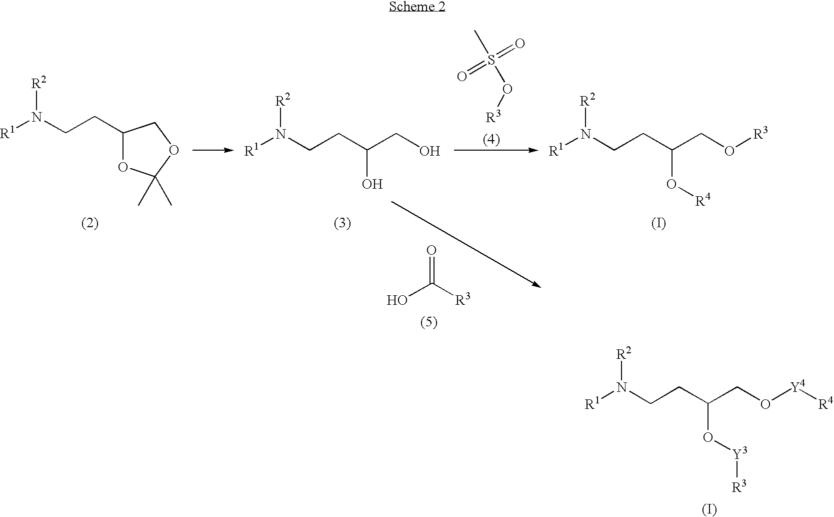

As shown in Scheme 2, compounds of Formula (3) can be prepared from compounds of Formula (2) by hydrolysis of the former using an acid such as but not limited to hydrochloric acid. The reaction can be performed immediately after the formation of (2) in the same reaction vessel. Compounds of Formula (I), wherein R¹, R², R³, and R⁴ are described herein, can be prepared by reacting compounds of Formula (3) with base, such as but not limited to sodium hydride, with a compound of Formula (4). The reaction is typically performed at an elevated temperature in a solvent such as but not limited to toluene.

If it is desired for R³ and R⁴ to be the same, two equivalents of (4) can be used. If R³ and R⁴ are to be different, one equivalent of (4) can be used to obtain a compound wherein R⁴ is H after purification. This intermediate can then be reacted with CH₃(SO₃)R⁴ to obtain a compound of Formula (I).

Compounds of Formula (I), wherein Y³ and Y⁴ are carbonyl, can be prepared from compounds of Formula (3) by coupling with an acid of Formula (5) under standard coupling conditions known in the art and widely available in the literature. If it is desired for R³ and R⁴ to be the same, two equivalents of (8) can be used. If R³ and R⁴ are to be different, one equivalent of (8) can be used to obtain a compound wherein R⁴ is H after purification. This intermediate can then be reacted with R⁴COOH to obtain a compound of Formula (I).

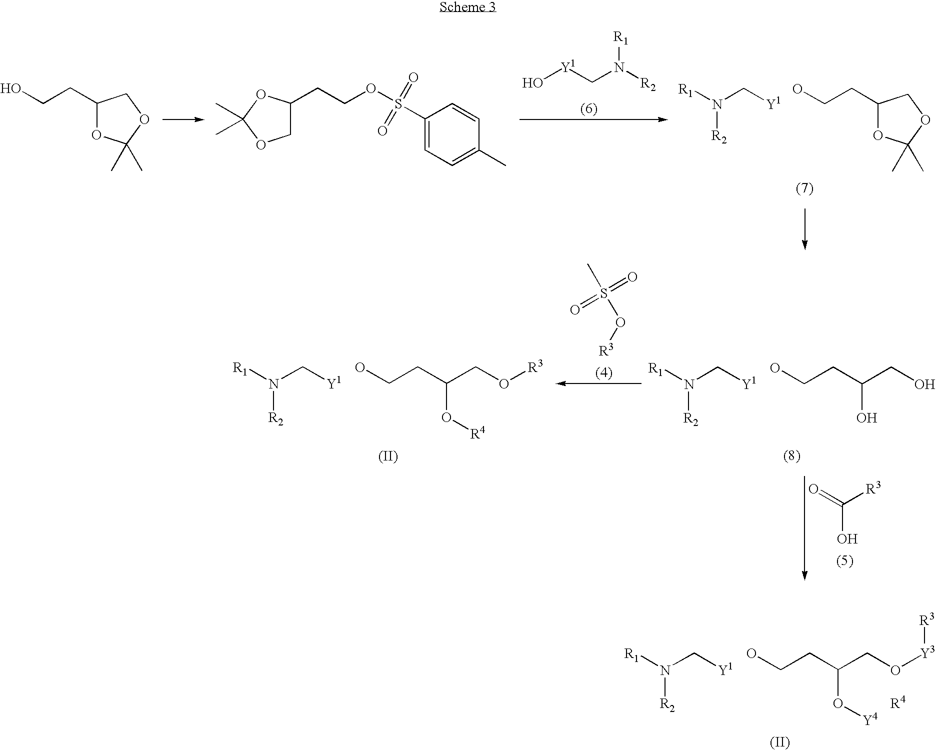

As shown in Scheme 3, 2-(2,2-dimethyl-1,3-dioxolan-4-yl)ethyl 4-methylbenzenesulfonate, which can be prepared as described in Example 1A, when reacted with an amino alcohol of Formula (6), wherein R¹, R², and Y¹ are as described herein, with a base such as but not limited to sodium hydride, will provide a compound of Formula (7).

Compounds of Formula (8) can be prepared from compounds of Formula (7) by hydrolysis of the latter using an acid such as aqueous hydrochloric acid in a solvent such as but not limited to tetrahydrofuran.

Compounds of Formula (II), wherein Y³ and Y⁴ are carbonyl, can be prepared from compounds of Formula (8) as described in Scheme 2.

Compounds of Formula (II), wherein Y³ and Y⁴ are a bond, can be prepared from compounds of Formula (8) as described in Scheme 2.

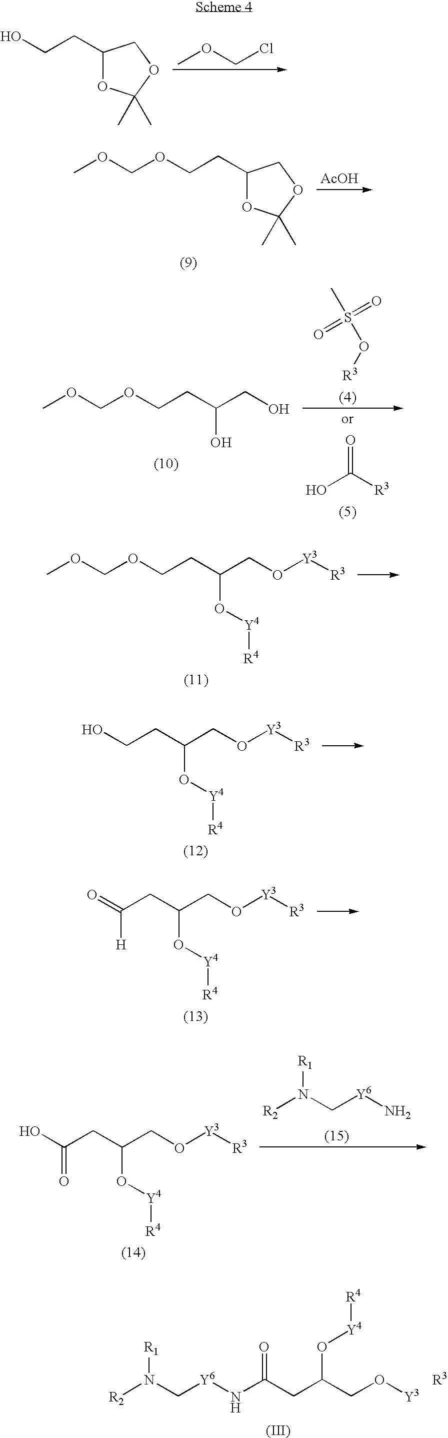

As shown in Scheme 4, compounds of Formula (9) can be prepared from 2-(2,2-dimethyl-1,3-dioxolan-4-yl)ethanol using chloromethyl methyl ether and a base such as but not limited to N-ethyl-N-isopropylpropan-2-amine in a solvent such as but not limited to dichloromethane. Compounds of Formula (10) can be prepared from compounds of Formula (9) by hydrolysis of the latter using an acid such as but not limited to acetic acid in a solvent such as water.

Compounds of Formula (11), wherein Y³ and Y⁴ are a bond wherein R³ and R⁴ are described herein, can be prepared by reacting compounds of Formula (10) with base, such as but not limited to sodium hydride, with a compound of Formula (4). The reaction is typically performed at an elevated temperature in a solvent such as but not limited to toluene.

If it is desired for R³ and R⁴ to be the same, two equivalents of (4) can be used. If R³ and R⁴ are to be different, one equivalent of (4) can be used to obtain a compound wherein R⁴ is H after purification. This intermediate can then be reacted with CH₃(SO₃)R⁴ to obtain a compound of Formula (I).

Compounds of Formula (11), wherein Y³ and Y⁴ are carbonyl, can be prepared from compounds of Formula (10) by coupling with an acid of Formula (5) under standard coupling conditions known in the art and widely available in the literature. If it is desired for R³ and R⁴ to be the same, two equivalents of (8) can be used. If R³ and R⁴ are to be different, one equivalent of (8) can be used to obtain a compound wherein R⁴ is H after purification. This intermediate can then be reacted with R⁴COOH to obtain a compound of Formula (III).

Compounds of Formula (12) can be prepared from compounds of Formula (11) by hydrolysis of the latter using an acid such as but not limited to hydrochloric acid.

Compounds of Formula (13) can be prepared from compounds of Formula (12) by oxidizing the latter using an oxidant such as but not limited to Dess-Martin Periodinane or any other oxidant known in the art or available in the literature.

Compounds of Formula (14) can be prepared from compounds of Formula (13) by using a Lindgren oxidation or some other suitable oxidation procedure known in the art and available in the literature.

Compounds of Formula (III) can be prepared by reacting compounds of Formula (14) with a compound of Formula (15) where R¹, R² and Y⁶ are defined herein, using standard coupling conditions known in the art and widely available in the literature.

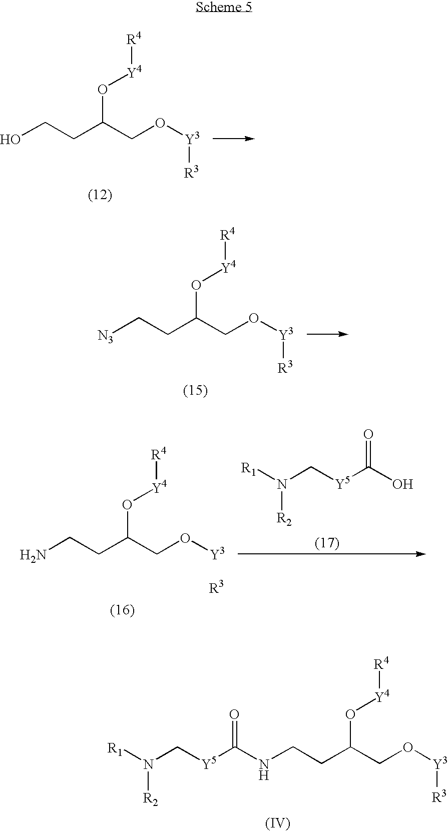

As shown in Scheme 5, compounds of Formula (15) can be prepared from compounds of Formula (12), wherein Y³, Y⁴, R³ and R⁴ are defined herein, using reagents such as DPPA (diphenylphosphoryl azide) and DEAD (diethyl azodicarboxylate) in a solvent such as but not limited to tetrahydrofuran. Compounds of Formula (16) can be prepared from compounds of Formula (15) by using a reagent such as triphenylphosphine in solvents such as but not limited to water and tetrahydrofuran. Compounds of Formula (IV) can be prepared from compounds of Formula (16) by coupling of an acid of Formula (17) wherein R¹, R² and Y⁵ are described herein, under standard coupling conditions known in the art and widely available in the literature.

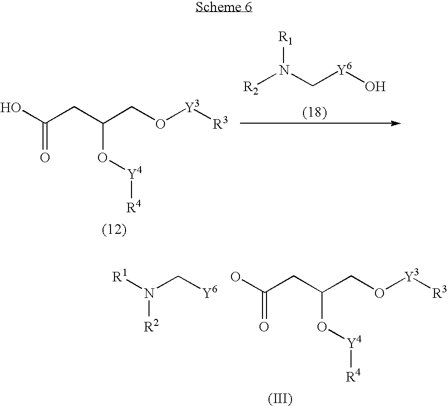

As shown in Scheme 6, compounds of Formula (III), wherein Y³, Y⁴, R³ and R⁴ are defined herein and Y² is O, can be prepared from compounds of Formula (12) by coupling of an amino alcohol of Formula (18) wherein R¹, R² and Y⁵ are described herein, under standard coupling conditions known in the art and widely available in the literature.

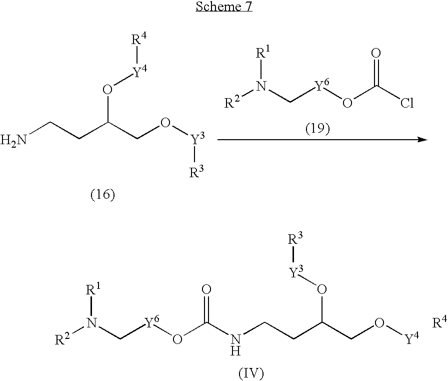

As shown in Scheme 7, compounds of Formula (IV), wherein Y² is NH, Y⁵ is O, can be prepared, wherein Y³, Y⁴, R³ and R⁴ are defined herein, by reacting compounds of Formula (16) with a chloroformate of Formula (19), wherein R¹, R² and Y⁶ are described herein, in a solvent such as but not limited to dichloromethane.

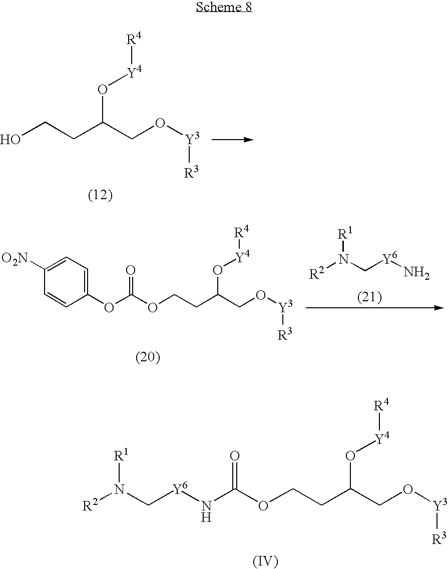

As shown in Scheme 8, compounds of Formula (20), wherein Y³, Y⁴, R³ and R⁴ are defined herein, can be prepared from 4-nitrophenyl chloroformate and compounds of Formula (12) in the presence of a base such as but not limited to N-ethyl-N-isopropylpropan-2-amine in a solvent such as dichloromethane. Compounds of Formula (IV), wherein Y² is O and Y⁵ is NH, can be prepared from compounds of Formula (20) by coupling with an amine of Formula (21), wherein R¹, R² and Y⁶ are described herein, in the presence of a base such as but not limited to N-methyl morpholine in a solvent such as but not limited to dichloromethane.

The following examples are presented to provide what is believed to be the most useful and readily understood description of procedures and conceptual aspects of this invention. The exemplified compounds were named using ACD/ChemSketch Version 5.06 (5 Jun. 2001, Advanced Chemistry Development Inc. Toronto, Ontario), or ChemDraw® Ver. 9.0.5 (CambridgeSoft, Cambridge, Mass.) except for Example 82, which was named using Marvin Version 5.1 (ChemAxon Kft., Budapest, Hungary). Intermediates were named using ChemDraw® Ver. 9.0.5 (CambridgeSoft, Cambridge, Mass.).

### Example 1

### 1-{3,4-bis[(9Z,12Z)-octadeca-9,12-dienyloxy]butyl}piperidine

### Example 1A

### 2-(2,2-dimethyl-1,3-dioxolan-4-yl)ethyl 4-methylbenzenesulfonate

2-(2,2-dimethyl-1,3-dioxolan-4-yl)ethanol (5 g) was added to dichloromethane (86 ml) and the mixture was cooled to 0° C. To this solution was added triethylamine (6.9 g, 9.6 ml), tosyl chloride (6.5 g) and 4-(dimethylamino)pyridine (0.42 g). The reaction was allowed to stir at room temperature overnight. The reaction was quenched with saturated NH₄Cl_{aq} and diluted with ethyl acetate. The aqueous layer was extracted twice with ethyl acetate and the combined organics were dried (Na₂SO₄), filtered, and concentrated by rotary evaporation. The residue was purified by flash column chromatography (Ethyl Acetate/Hexanes 0-100%, Analogix) to afford the title compound. MS (ESI) m/z 300.9 (M+H)⁺; ¹H NMR (400 MHz, CDCl₃) δ 7.79 (d, J=8.29 Hz, 2H) 7.35 (d, J=7.98 Hz, 2H) 4.06-4.23 (m, 3H) 4.01 (dd, J=7.98, 6.14 Hz, 1H) 3.51 (dd, J=8.13, 6.90 Hz, 1H) 2.45 (s, 3H) 1.82-1.98 (m, 2H) 1.31 (d, J=18.72 Hz, 6H).

### Example 1B

### 1-{3,4-bis[(9Z,12Z)-octadeca-9,12-dienyloxy]butyl}piperidine

2-(2,2-dimethyl-1,3-dioxolan-4-yl)ethyl 4-methylbenzenesulfonate (Example 1A, 500 mg) was placed in a microwave reaction vial along with piperidine (1-2 eq.), Hunig's base (2 eq) and dioxane (2.2 ml). The reaction was placed in the microwave (Biotage Initiator) for 15 minutes at 140° C. After TLC analysis confirmed completion, 4N HCl (4 ml) was added until the mixture was acidic and the reaction was stirred overnight at room temperature. 6N NaOH was then added until the mixture was basic, and the mixture was diluted with water and extracted (5×, chloroform). The combined organics were dried (Na₂SO₄), filtered and concentrated by rotary evaporation. The hydrolysis of the acetonide was confirmed by ¹H NMR of the crude material in each case. The residue was taken up in toluene (0.2-0.3 M) and NaH (5-10 eq) was added portionwise, slowly at first. The flask was purged with nitrogen and the reaction was allowed to stir for 45 minutes. (9Z,12Z)-octadeca-9,12-dienyl methanesulfonate (Nu-Check Prep, 2.5 eq) was added via syringe and the reaction was stirred at 80-90° C. for 3-4 hours or overnight. Ethanol was added slowly dropwise to quench excess sodium hydride and then water was added carefully. After diluting with ethyl acetate and water, the aqueous layer was separated and then extracted with ethyl acetate (3×). The combined organics were dried (Na₂SO₄), filtered and concentrated by rotary evaporation. The residue was purified by flash column chromatography (Ethyl Acetate/Hexanes 0-100%, Analogix) to afford the title compound. MS (ESI) m/z 670.7 (M+H)⁺; ¹H NMR (300 MHz, CDCl₃) δ 5.24-5.46 (m, 8H) 3.50-3.62 (m, 1H) 3.34-3.48 (m, 6H) 2.77 (t, J=5.95 Hz, 4H) 2.30-2.46 (m, 6H) 2.05 (q, J=6.48 Hz, 8H) 1.48-1.75 (m, 8H) 1.21-1.46 (m, 36H) 0.81-0.95 (m, 6H).

### Example 2

### 4-{3,4-bis[(9Z,12Z)-octadeca-9,12-dienyloxy]butyl}morpholine

Example 2 was prepared using the procedure described in Example 1B, substituting morpholine for piperidine. MS (ESI) m/z 672.6 (M+H)⁺; ¹H NMR (300 MHz, CDCl₃) δ 5.26-5.45 (m, 8H) 3.71 (t, J=4.75 Hz, 4H) 3.52-3.64 (m, 1H) 3.33-3.50 (m, 6H) 2.77 (t, J=5.76 Hz, 4H) 2.34-2.52 (m, 6H) 2.05 (q, J=6.56 Hz, 8H) 1.48-1.80 (m, 8H) 1.22-1.42 (m, 30H) 0.85-0.94 (m, 6H).

### Example 3

### N,N-diethyl-3,4-bis[(9Z,12Z)-octadeca-9,12-dienyloxy]butan-1-amine

Example 3 was prepared using the procedure described in Example 1B, substituting diethyl amine for piperidine. MS (ESI) m/z 658.6 (M+H)⁺; ¹H NMR (300 MHz, CDCl₃) δ ppm 5.24-5.47 (m, 8H) 3.52-3.65 (m, 1H) 3.35-3.49 (m, 6H) 2.77 (t, J=5.93 Hz, 4H) 2.42-2.64 (m, 6H) 1.98-2.12 (m, 8H) 1.48-1.75 (m, 8H) 1.21-1.44 (m, 30H) 1.02 (t, J=7.12 Hz, 6H) 0.82-0.95 (m, 6H).

### Example 4

### N,N-dimethyl-3,4-bis[(9Z,12Z)-octadeca-9,12-dienyloxy]butan-1-amine

Example 4 was prepared using the procedure described in Example 1B, substituting dimethyl amine hydrochloride for piperidine. MS (ESI) m/z 630.6 (M+H)⁺; ¹H NMR (300 MHz, CDCl₃) δ 5.24-5.47 (m, 8H) 3.51-3.66 (m, 1H) 3.34-3.50 (m, 6H) 2.77 (t, J=5.95 Hz, 4H) 2.36 (t, J=7.54 Hz, 2H) 2.22 (s, 6H) 1.98-2.11 (m, 8H) 1.46-1.76 (m, 5H) 1.22-1.42 (m, 32H) 0.82-0.94 (m, 6H).

### Example 5

### 1-{3,4-bis[(9Z,12Z)-octadeca-9,12-dienyloxy]butyl}-4-phenylpiperazine

Example 5 was prepared using the procedure described in Example 1B, substituting 4-phenylpiperazine for piperidine. MS (ESI) m/z 747.6 (M+H)⁺; ¹H NMR (300 MHz, CDCl₃) δ 7.26 (t, J=6.0, 2H) 6.93 (d, J=8.72 Hz, 2H) 6.78-6.96 (m, 3H) 5.24-5.46 (m, 8H) 3.54-3.66 (m, 1H) 3.35-3.53 (m, 6H) 3.20 (t, J=4.96 Hz, 4H) 2.77 (t, J=5.95 Hz, 4H) 2.55-2.67 (m, 4H) 2.50 (t, J=7.54 Hz, 2H) 2.05 (q, J=6.48 Hz, 8H) 1.46-1.85 (m, 4H) 1.20-1.42 (m, 32H) 0.80-0.96 (m, 6H).

### Example 6

### 1-{3,4-bis[(9Z,12Z)-octadeca-9,12-dienyloxy]butyl}-4-methylpiperazine

Example 6 was prepared using the procedure described in Example 1B, substituting 4-methylpiperazine for piperidine. MS (ESI) m/z 685.6 (M+H)⁺; ¹H NMR (300 MHz, CDCl₃) δ 5.23-5.46 (m, 8H) 3.50-3.66 (m, 1H) 3.33-3.50 (m, 6H) 2.72-2.82 (m, 4H) 2.36-2.59 (m, J=6.74 Hz, 10H) 2.29 (s, 3H) 1.98-2.12 (m, J=6.35, 6.35 Hz, 8H) 1.47-1.78 (m, 6H) 1.21-1.42 (m, 32H) 0.81-0.94 (m, 6H).

### Example 7

### N-(2-methoxyethyl)-N-methyl-3,4-bis[(9Z,12Z)-octadeca-9,12-dienyloxy]butan-1-amine

Example 7 was prepared using the same procedure described in Example 1B, substituting 2-methoxy-N-methylethanamine for piperidine. MS (ESI) m/z 674.6 (M+H)⁺; ¹H NMR (300 MHz, CDCl₃) δ 5.22-5.46 (m, 8H) 3.52-3.64 (m, 1H) 3.36-3.51 (m, 6H) 3.35 (s, 3H) 2.77 (t, J=6.15 Hz, 4H) 2.54-2.61 (m, 2H) 2.45-2.54 (m, 2H) 2.27 (s, 3H) 1.98-2.10 (m, 8H) 1.22-1.74 (m, 40H) 0.83-0.93 (m, 6H).

### Example 8

### 1-{3,4-bis[(9Z,12Z)-octadeca-9,12-dienyloxy]butyl}-4-(2-methoxyphenyl)piperazine

Example 8 was prepared using the same procedure described in Example 1B, substituting 1-(2-methoxyphenyl)piperazine for piperidine. ¹H NMR (300 MHz, CDCl₃) δ 6.80-7.06 (m, 4H) 5.24-5.47 (m, 8H) 3.86 (s, 3H) 3.52-3.65 (m, 1H) 3.34-352 (m, 6H) 2.99-3.18 (m, 4H) 2.77 (t, J=5.93 Hz, 4H) 2.58-2.71 (m, J=4.41 Hz, 4H) 2.46-2.57 (m, 2H) 2.05 (q, J=6.56 Hz, 8H) 1.49-1.80 (m, 4H) 1.22-1.41 (m, 34H) 0.84-0.94 (m, 6H).

### Example 9

### N-{3,4-bis[(9Z,12Z)-octadeca-9,12-dienyloxy]butyl}-N,N′,N′-trimethylethane-1,2-diamine

Example 9 was prepared using the same procedure in Example 1B, substituting N′,N′,N²-trimethylethane-1,2-diamine for piperidine. MS (ESI) m/z 687.7 (M+H)⁺; ¹H NMR (300 MHz, CDCl₃) δ 5.25-5.46 (m, 8H) 3.52-3.65 (m, 1H) 3.34-3.50 (m, 6H) 2.77 (t, J=5.93 Hz, 4H) 2.59-2.73 (m, 6H) 2.34-2.43 (m, 6H) 2.19 (s, 3H) 2.05 (q, J=6.56 Hz, 8H) 1.64-1.87 (m, 2H) 1.46-1.61 (m, 2H) 1.16-1.42 (m, 34H) 0.83-0.95 (m, 6H).

### Example 10

### N-{3,4-bis[(9Z,12Z)-octadeca-9,12-dienyloxy]butyl}-N-methyl-N-(2-pyridin-2-ylethyl)amine

Example 10 was prepared using the same procedure in Example 1B, substituting N-methyl-2-(pyridin-2-yl)ethanamine for piperidine. MS (ESI) m/z 721.6 (M+H)⁺; ¹H NMR (300 MHz, CDCl₃) δ 8.51 (d, J=4.75 Hz, 1H) 7.58 (td, J=7.63, 1.70 Hz, 1H) 7.19 (d, J=7.80 Hz, 1H) 7.10 (dd, J=6.95, 5.59 Hz, 1H) 5.24-5.44 (m, 8H) 3.50-3.60 (m, 1H) 3.32-3.46 (m, 6H) 2.92-3.06 (m, J=6.78 Hz, 2H) 2.80-2.92 (m, 2H) 2.77 (t, J=6.10 Hz, 4H) 2.51-2.64 (m, J=4.07 Hz, 1H) 2.35 (s, 2H) 2.05 (q, J=6.56 Hz, 8H) 1.44-1.80 (m, 4H) 1.21-1.42 (m, 34H) 0.84-0.93 (m, 6H).

### Example 11

### N-benzyl-N-{3,4-bis[(9Z,12Z)-octadeca-9,12-dienyloxy]butyl}-N-methylamine

Example 11 was prepared using the same procedure in Example 1B, substituting N-methyl-1-phenylmethanamine for piperidine. MS (ESI) m/z 706.6 (M+H)⁺; ¹H NMR (300 MHz, CDCl₃) δ 7.19-7.33 (m, 5H) 5.27-5.44 (m, 8H) 3.31-3.69 (m, 9H) 2.77 (t, J=5.93 Hz, 4H) 2.39-2.56 (m, 2H) 2.17 (s, 3H) 2.05 (q, J=6.22 Hz, 8H) 1.42-1.81 (m, 4H) 1.21-1.43 (m, 34H) 0.82-0.95 (m, 6H).

### Example 12

### N-{3,4-bis[(9Z,12Z)-octadeca-9,12-dienyloxy]butyl}-N-(4-fluorobenzyl)-N-methylamine

Example 12 was prepared using the same procedure in Example 1B, substituting 1-(4-fluorophenyl)-N-methylmethanamine for piperidine. MS (ESI) m/z 724.5 (M+H)⁺; ¹H NMR (300 MHz, CDCl₃) δ 7.17-7.33 (m, 2H) 6.87-7.08 (m, 2H) 5.22-5.48 (m, 3.30-3.63 (m, 7H) 2.77 (t, J=5.95 Hz, 4H) 2.38-2.54 (m, 2H) 2.16 (s, 3H) 2.05 (q, J=6.48 Hz, 8H) 1.43-1.81 (m, 4H) 1.20-1.43 (m, 34H) 0.82-0.95 (m, 6H).

### Example 13

### 1-{3,4-bis[(9Z,12Z)-octadeca-9,12-dienyloxy]butyl}-4-(2-fluorophenyl)piperazine

Example 13 was prepared using the same procedure in Example 1B, substituting 1-(2-fluorophenyl)piperazine for piperidine. MS (ESI) m/z 765.5 (M+H)⁺; ¹H NMR (300 MHz, CDCl₃) δ 6.85-7.13 (m, 4H) 5.24-5.47 (m, 8H) 3.53-3.66 (m, 1H) 3.36-3.52 (m, 6H) 3.11 (t, J=4.75 Hz, 4H) 2.77 (t, J=5.93 Hz, 4H) 2.59-2.69 (m, 4H) 2.45-2.56 (m, 2H) 2.05 (q, J=6.56 Hz, 8H) 1.49-1.79 (m, 4H) 1.20-1.42 (m, 34H) 0.79-0.96 (m, 6H).

### Example 14

### N-benzyl-N-{3,4-bis[(9Z,12Z)-octadeca-9,12-dienyloxy]butyl}-N-ethylamine

Example 14 was prepared using the same procedure in Example 1B, substituting N-benzylethanamine for piperidine. MS (ESI) m/z 720.6 (M+H)⁺; ¹H NMR (300 MHz, CDCl₃) δ 7.11-7.41 (m, 5H) 5.24-5.49 (m, 8H) 3.27-3.63 (m, 9H) 2.77 (t, J=5.95 Hz, 4H) 2.42-2.62 (m, 4H) 2.05 (q, J=6.21 Hz, 8H) 1.43-1.78 (m, 4H) 1.20-1.43 (m, 34H) 1.03 (t, J=7.14 Hz, 3H) 0.82-0.94 (m, 6H).

### Example 15

### N-{3,4-bis[(9Z,12Z)-octadeca-9,12-dienyloxy]butyl}-N-ethyl-N′,N′-dimethylethane-1,2-diamine

Example 15 was prepared using the same procedure in Example 1B, substituting N¹-ethyl-N²,N²-dimethylethane-1,2-diamine for piperidine. MS (ESI) m/z 701.6 (M+H)⁺; ¹H NMR (300 MHz, CDCl₃) δ 5.24-5.46 (m, 8H) 3.51-3.64 (m, 1H) 3.34-3.48 (m, 6H) 2.70-2.83 (m, 4H) 2.45-2.63 (m, 6H) 2.38 (t, J=7.34 Hz, 2H) 2.24 (s, 6H) 2.05 (q, J=6.21 Hz, 8H) 1.47-1.70 (m, 4H) 1.22-1.41 (m, 34H) 1.02 (t, J=7.14 Hz, 3H) 0.79-0.94 (m, 6H).

### Example 16

### 1-{3,4-bis[(9Z,12Z)-octadeca-9,12-dienyloxy]butyl}-N,N-dimethylpiperidin-4-amine

Example 16 was prepared using the same procedure in Example 1B, substituting N,N-dimethylpiperidin-4-amine for piperidine. MS (ESI) m/z 713.6 (M+H)⁺; ¹H NMR (300 MHz, CDCl₃) δ 5.25-5.46 (m, 8H) 3.51-3.63 (m, 1H) 3.33-3.49 (m, 6H) 2.99 (s, 4H) 2.80-2.91 (m, J=16.26 Hz, 1H) 2.77 (t, J=5.95 Hz, 4H) 2.39-2.53 (m, 2H) 2.34 (s, 6H) 1.96-2.12 (m, 8H) 1.46-1.91 (m, 10H) 1.18-1.43 (m, 34H) 0.80-0.96 (m, 6H).

### Example 17

### 1-{3,4-bis[(9Z,12Z)-octadeca-9,12-dienyloxy]butyl}-N,N-dimethylpyrrolidin-3-amine

Example 17 was prepared using the same procedure in Example 1B, substituting N,N-dimethylpyrrolidin-3-amine for piperidine. MS (ESI) m/z 699.6 (M+H)⁺; ¹H NMR (300 MHz, CDCl₃) δ 5.25-5.46 (m, 8H) 3.50-3.63 (m, 1H) 3.33-3.50 (m, 6H) 2.71-2.93 (m, 7H) 2.40-2.65 (m, 3H) 2.25-2.36 (m, 1H) 2.22 (s, 6H) 2.05 (q, J=6.56 Hz, 8H) 1.78-2.00 (m, 3H) 1.44-1.79 (m, 5H) 1.22-1.42 (m, 34H) 0.82-0.96 (m, 6H).

### Example 18

### N,N-bis(2-methoxyethyl)-3,4-bis [(9Z,12Z)-octadeca-9,12-dienyloxy]butan-1-amine

Example 18 was prepared using the same procedure in Example 1B, substituting bis(2-methoxyethyl)amine for piperidine. MS (ESI) m/z 718.5 (M+H)⁺; ¹H NMR (300 MHz, CDCl₃) δ 5.24-5.47 (m, 8H) 3.50-3.66 (m, 1H) 3.37-3.50 (m, 10H) 3.34 (s, 6H) 2.54-2.82 (m, 10H) 2.05 (q, J=6.35 Hz, 8H) 1.47-1.74 (m, 4H) 1.23-1.43 (m, 34H) 0.82-0.95 (m, 6H).

### Example 19

### 1-{3,4-bis[(9Z,12Z)-octadeca-9,12-dienyloxy]butyl}-4-methoxypiperidine

Example 18 was prepared using the same procedure in Example 1B, substituting 4-methoxypiperidine for piperidine. MS (ESI) m/z 700.5 (M+H)⁺; ¹H NMR (300 MHz, CDCl₃) δ 5.19-5.49 (m, 8H) 3.49-3.65 (m, 1H) 3.36-3.49 (m, 6H) 3.33 (s, 3H) 3.13-3.27 (m, 1H) 2.66-2.83 (m, 6H) 2.35-2.50 (m, 2H) 1.84-2.21 (m, 11H) 1.46-1.78 (m, 7H) 1.20-1.43 (m, 34H) 0.83-0.92 (m, 6H).

### Example 20

### 1-{(3R)-3,4-bis[(9Z,12Z)-octadeca-9,12-dienyloxy]butyl}pyrrolidine

### Example 20A

### (R)-2-(2,2-dimethyl-1,3-dioxolan-4-yl)ethyl 4-methylbenzenesulfonate

(R)-2-(2,2-dimethyl-1,3-dioxolan-4-yl)ethanol (10 g) was taken up in dichloromethane (171 ml) and cooled to 0° C. To this solution was added triethylamine (13.8 g), tosyl chloride (13.0 g) and for 4-(dimethylamino)pyridine (0.84 g). The reaction was allowed to stir at room temperature overnight. The reaction was quenched with sat. NH₄Cl_{aq} and diluted with ethyl acetate. The aqueous layer was extracted twice with ethyl acetate and the combined organics were dried (Na₂SO₄), filtered, and concentrated by rotary evaporation. The residue was purified by flash column chromatography (Ethyl Acetate/Hexanes 0-100%, Analogix) to afford the title compound. MS (ESI) m/z 300.9 (M+H)⁺; ¹H NMR (400 MHz, CDCl₃) δ 7.79 (d, J=8.29 Hz, 2H) 7.35 (d, J=7.98 Hz, 2H) 4.06-4.23 (m, 3H) 4.01 (dd, J=7.98, 6.14 Hz, 1H) 3.51 (dd, J=8.13, 6.90 Hz, 1H) 2.45 (s, 3H) 1.82-1.98 (m, 2H) 1.31 (d, J=18.72 Hz, 6H); [α]²⁰=−13 (c 1.6, CHCl₃).

### Example 20B

### 1-{(3R)-3,4-bis[(9Z,12Z)-octadeca-9,12-dienyloxy]butyl}pyrrolidine

Example 20B was prepared using the same procedure in Example 1B, substituting Example 20A for Example 1A and pyrrolidine for piperidine. MS (ESI) m/z 656.6 (M+H)⁺; ¹H NMR (300 MHz, CDCl₃) δ 5.25-5.45 (m, 8H) 3.51-3.63 (m, 1H) 3.33-3.51 (m, 6H) 2.77 (t, J=6.10 Hz, 4H) 2.44-2.58 (m, 6H) 2.05 (q, J=6.55 Hz, 8H) 1.48-1.82 (m, 10H) 1.20-1.44 (m, 34H) 0.84-0.96 (m, 6H).

### Example 21

### 1-{(3S)-3,4-bis[(9Z,12Z)-octadeca-9,12-dienyloxy]butyl}pyrrolidine

### Example 21A

### (S)-2-(2,2-dimethyl-1,3-dioxolan-4-yl)ethyl 4-methylbenzenesulfonate

(S)-2-(2,2-dimethyl-1,3-dioxolan-4-yl)ethanol (10 g) was taken up in dichloromethane (171 ml) and cooled to 0° C. To this solution was added triethylamine (13.8 g), tosyl chloride (13.0 g, 68.4 mmol) and then 4-(dimethylamino)pyridine (0.84 g). The reaction was allowed to stir at room temperature overnight. The reaction was quenched with sat. NH₄Cl_{aq} and diluted with ethyl acetate. The aqueous layer was extracted twice with ethyl acetate and the combined organics were dried (Na₂SO₄), filtered, and concentrated by rotary evaporation. The residue was purified by flash column chromatography (Ethyl Acetate/Hexanes 0-100%, Analogix) to afford the title compound. MS (ESI) m/z 300.9 (M+H)⁺; ¹H NMR (300 MHz, CDCl₃) δ 7.79 (d, J=8.29 Hz, 2H) 7.35 (d, J=7.98 Hz, 2H) 4.06-4.23 (m, 3H) 4.01 (dd, J=7.98, 6.14 Hz, 1H) 3.51 (dd, J=8.13, 6.90 Hz, 1H) 2.45 (s, 3H) 1.82-1.98 (m, 2H) 1.31 (d, J=18.72 Hz, 6H); [α]²⁰=+13 (c 1.2, CHCl₃).

### Example 21B

### 1-{(3 S)-3,4-bis[(9Z,12Z)-octadeca-9,12-dienyloxy]butyl}pyrrolidine

Example 21B was prepared using the same procedure in Example 1B, substituting Example 21A for Example 1A and pyrrolidine for piperidine. MS (ESI) m/z 656.5 (M+H)⁺; ¹H NMR (300 MHz, CDCl₃) δ 5.25-5.45 (m, 8H) 3.51-3.63 (m, 1H) 3.33-3.51 (m, 6H) 2.77 (t, J=6.10 Hz, 4H) 2.44-2.58 (m, 6H) 2

### Example 22

### N-{(3R)-3,4-bis[(9Z,12Z)-octadeca-9,12-dienyloxy]butyl}-N,N-diethylamine

Example 22 was prepared using the same procedure in Example 1B, substituting Example 20A for Example 1A and diethyl amine for piperidine. MS (ESI) m/z 658.6 (M+H)⁺; ¹H NMR (300 MHz, CDCl₃) δ 5.24-5.47 (m, 8H) 3.52-3.65 (m, 1H) 3.35-3.49 (m, 6H) 2.77 (t, J=5.93 Hz, 4H) 2.42-2.64 (m, 6H) 1.98-2.12 (m, 8H) 1.48-1.75 (m, 8H) 1.21-1.44 (m, 30H) 1.02 (t, J=7.12 Hz, 6H) 0.82-0.95 (m, 6H).

### Example 23

### N-{(3S)-3,4-bis[(9Z,12Z)-octadeca-9,12-dienyloxy]butyl}-N,N-diethylamine

Example 23 was prepared using the same procedure in Example 1B, substituting Example 21A for Example 1A and diethyl amine for piperidine. MS (ESI) m/z 658.6 (M+H)⁺; ¹H NMR (300 MHz, CDCl₃) δ 5.24-5.47 (m, 8H) 3.52-3.65 (m, 1H) 3.35-3.49 (m, 6H) 2.77 (t, J=5.93 Hz, 4H) 2.42-2.64 (m, 6H) 1.98-2.12 (m, 8H) 1.48-1.75 (m, 8H) 1.21-1.44 (m, 30H) 1.02 (t, J=7.12 Hz, 6H) 0.82-0.95 (m, 6H).

### Example 24

### 1-{3,4-bis[(9Z,12Z)-octadeca-9,12-dienyloxy]butyl}pyrrolidine

Example 24 was prepared using the same procedure in Example 1B, substituting pyrrolidine for piperidine. MS (ESI) m/z 656.6 (M+H)⁺; ¹H NMR (300 MHz, CDCl₃) δ 5.25-5.45 (m, 8H) 3.51-3.63 (m, 1H) 3.33-3.51 (m, 6H) 2.77 (t, J=6.10 Hz, 2.44-2.58 (m, 6H) 2.05 (q, J=6.55 Hz, 8H) 1.48-1.82 (m, 10H) 1.20-1.44 (m, 34H) 0.84-0.96 (m, 6H).

### Example 25

### N-(2-{3,4-bis[(9Z,12Z)-octadeca-9,12-dienyloxy]butoxy}ethyl)-N,N-diethylamine

Example 25 was prepared as described in Example 31 using the appropriate reagents and conditions. MS (ESI) m/z 702.7 (M+H)⁺; ¹H NMR (300 MHz, CHLOROFORM-D) δ ppm 5.27-5.44 (m, 8H) 3.36-3.66 (m, 11H) 2.77 (t, J=5.93 Hz, 4H) 2.64 (t, J=6.61 Hz, 2H) 2.57 (q, J=7.12 Hz, 4H) 2.00-2.10 (m, 8H) 1.67-1.83 (m, 2H) 1.25-1.41 (m, 36H) 1.03 (t, J=7.12 Hz, 6H) 0.85-0.93 (m, 6H).

### Example 26

### 2-(2-{3,4-bis[(9Z,12Z)-octadeca-9,12-dienyloxy]butoxy}ethyl)-1-methylpyrrolidine

Example 26 was prepared as described in Example 31 using the appropriate reagents and conditions. MS (ESI) m/z 714.6 (M+H)⁺; ¹H NMR (400 MHz, CHLOROFORM-D) δ ppm 5.27-5.43 (m, 8H) 3.37-3.64 (m, 11H) 3.03-3.14 (m, 1H) 2.77 (t, J=6.44 Hz, 4H) 2.27-2.37 (m, 3H) 2.16 (q, J=9.21 Hz, 2H) 2.05 (q, J=6.75 Hz, 8H) 1.90-2.00 (m, 4H) 1.63-1.85 (m, 4H) 1.23-1.41 (m, 36H) 0.83-0.94 (m, 6H).

### Example 27

### 1-(2-{3,4-bis[(9Z,12Z)-octadeca-9,12-dienyloxy]butoxy}ethyl)aziridine

Example 27 was prepared as described in Example 31 using the appropriate reagents and conditions. MS (ESI) m/z 672.6 (M+H)⁺; ¹H NMR (400 MHz, CHLOROFORM-D) δ ppm 5.26-5.45 (m, 8H) 3.48-3.64 (m, 6H) 3.36-3.47 (m, 5H) 2.77 (t, J=6.44 Hz, 4H) 2.27-2.46 (m, 2H) 2.05 (q, J=6.96 Hz, 8H) 1.77 (none, 1H) 1.67-1.87 (m, 2H) 1.48-1.61 (m, 4H) 1.23-1.41 (m, 36H) 0.82-0.95 (m, 6H).

### Example 28

### 1-(2-{3,4-bis[(9Z,12Z)-octadeca-9,12-dienyloxy]butoxy}ethyl)-4-methylpiperazine

Example 28 was prepared as described in Example 31 using the appropriate reagents and conditions. MS (ESI) m/z 729.6 (M+H); ¹H NMR (400 MHz, CHLOROFORM-D) δ ppm 5.26-5.44 (m, 8H) 3.46-3.63 (m, 6H) 3.35-3.47 (m, 5H) 2.73-2.81 (m, 4H) 2.58 (t, J=5.98 Hz, 2H) 2.37-2.57 (m, 4H) 2.28 (s, 3H) 2.05 (q, J=6.96 Hz, 8H) 1.75-1.86 (m, 2H) 1.48-1.60 (m, 4H) 1.21-1.40 (m, 36H) 0.84-0.92 (m, 6H).

### Example 29

### N-(2-{3,4-bis[(9Z,12Z)-octadeca-9,12-dienyloxy]butoxy}ethyl)-N,N-dimethylamine

Example 30 was prepared as described in Example 31 using the appropriate reagents and conditions. MS (ESI) m/z 674.6 (M+H); ¹H NMR (400 MHz, CHLOROFORM-D) δ ppm 5.25-5.43 (m, 8H) 3.46-3.63 (m, 6H) 3.35-3.47 (m, 5H) 2.77 (t, J=6.4 Hz, 4H) 2.49 (t, J=5.98 Hz, 2H) 2.22-2.30 (m, 6H) 2.05 (q, J=6.75 Hz, 8H) 1.67-1.87 (m, 2H) 1.49-1.60 (m, 4H) 1.21-1.41 (m, 32H) 0.89 (t, J=6.75 Hz, 6H).

### Example 30

### 4-(diethylamino)-2-[(9Z,12Z)-octadeca-9,12-dienoyloxy]butyl(9Z,12Z)-octadeca-9,12-dienoate

### Example 30A

### 4-(diethylamino)butane-1,2-diol

Example 1A (500 mg, 1.66 mmol), diethylamine (609 mg, 8.32 mmol) and dioxane (2.5 mL) were combined in a microwave vial. The vial was capped and heated at 150° C. for 20 minutes in a Biotage Initiator microwave. The reaction was diluted with ethyl acetate and water. The layers were separated and the aqueous layer was extracted (3×) with ethyl acetate. The combined organics were dried (Na₂SO₄), filtered and concentrated by rotary evaporation. The residue was of sufficient purity that it was used in the next step without purification. MS (ESI) m/z 201.2 (M+H); ¹H NMR (300 MHz, CHLOROFORM-D) δ ppm 4.02-4.17 (m, 2H) 3.53 (t, J=7.34 Hz, 1H) 2.39-2.62 (m, 6H) 1.72-1.87 (m, 1H) 1.58-1.72 (m, 1H) 1.41 (s, 3H) 1.35 (s, 3H) 1.02 (t, J=7.14 Hz, 6H).

### Example 30B

### 4-(diethylamino)butane-1,2-diol

Example 30A (335 mg, 1.66 mmol) was dissolved in tetrahydrofuran and 2N HCl_{(aq)} (8.5 mL each) and stirred overnight at room temperature. The acidic solution was made basic by the addition of 5 N NaOH (aq). The aqueous layer was extracted with chloroform (6×) and the combined organics were dried (MgSO₄), filtered and concentrated by rotary evaporation. The residue was used in the next step without further purification. ¹H NMR (300 MHz, CHLOROFORM-D) δ ppm 3.85-3.94 (m, 1H) 3.56-3.63 (m, 1H) 3.44-3.51 (m, 1H) 2.51-2.83 (m, 6H) 2.32-2.48 (m, 2H) 1.69-1.84 (m, 1H) 1.41-1.57 (m, 1H) 1.06 (t, J=7.14 Hz, 6H).

### Example 30C

### 4-(diethylamino)-2-[(9Z,12Z)-octadeca-9,12-dienoyloxy]butyl (9Z,12Z)-octadeca-9,12-dienoate

Example 30B (75 mg, 0.465 mmol) was dissolved in dichloromethane (2.3 ml). (9Z,12Z)-octadeca-9,12-dienoic acid (261 mg, 0.93 mmol) was added followed by 1-ethyl-3-[3-(dimethylamino)propyl]-carbodiimide hydrochloride (187 mg, 0.977 mmol) and 4-dimethylaminopyridine (5.7 mg, 0.047 mmol). The reaction was stirred overnight at room temperature. The reaction was quenched with water and diluted with ethyl acetate. The aqueous layer was extracted with ethyl acetate (3×). The combined organics were dried (Na₂SO₄), filtered and concentrated by rotary evaporation. The residue was purified by regular phase chromatography (Analogix, 0-100% hexanes:ethyl acetate) to give Example 30. MS (ESI) m/z 686.5 (M+H); ¹H NMR (300 MHz, CHLOROFORM-D) δ ppm 5.26-5.45 (m, 8H) 5.13 (dd, J=6.78, 3.39 Hz, 1H) 4.26 (dd, J=1 1.87, 3.39 Hz, 1H) 4.07 (dd, J=11.87, 6.10 Hz, 1H) 2.77 (t, J=5.76 Hz, 4H) 2.42-2.55 (m, 6H) 2.30 (t, J=7.46 Hz, 4H) 2.05 (q, J=6.56 Hz, 8H) 1.68-1.79 (m, 2H), 1.23-1.42 (m, 32H) 1.00 (t, J=7.12 Hz, 6H) 0.83-0.93 (m, 6H).

### Example 31

### 1-(2-{3,4-bis[(9Z,12Z)-octadeca-9,12-dienyloxy]butoxy}ethyl)pyrrolidine

### Example 31A

### 1-(2-(2-(2,2-dimethyl-1,3-dioxolan-4-yl)ethoxy)ethyl)pyrrolidine

To a suspension of sodium hydride (167 mg, 6.95 mmol) in tetrahydrofuran (3 mL) at 0° C. was added a tetrahydrofuran (3 mL) solution of 2-(pyrrolidin-1-yl)ethanol (200 mg, 1.7 mmol). The mixture was warmed to room temperature with stirring for 1 hour and then cooled back down to 0° C. A tetrahydrofuran solution of 2-(2,2-dimethyl-1,3-dioxolan-4-yl)ethyl 4-methylbenzenesulfonate (522 mg, 1.7 mmol) was added dropwise and the mixture was stirred overnight under nitrogen. The reaction was worked up with ethanol and then with water. More water was added along with ethyl acetate and the layers were separated. The aqueous layer was extracted (2×) with ethyl acetate and the combined organics were dried (Na₂SO₄), filtered and concentrated. The residue was purified by regular phase chromatography (Analogix, 0-100% hexanes:ethyl acetate) to give Example 31A. MS (ESI) m/z 243.9 (M+H); ¹H NMR (300 MHz, CHLOROFORM-D) δ ppm 4.12-4.26 (m, 1H) 4.06 (dd, J=7.97, 5.93 Hz, 1H) 3.44-3.67 (m, 5H) 2.66 (t, J=6.10 Hz, 2H) 2.48-2.60 (m, 4H) 1.69-1.96 (m, 6H) 1.40 (s, 3H) 1.35 (s, 3H).

### Example 31B

### 1-(2-{3,4-bis[(9Z,12Z)-octadeca-9,12-dienyloxy]butoxy}ethyl)pyrrolidine

Example 31A (423 mg, 1.7 mmol) was dissolved in tetrahydrofuran (1-2 mL) and 4N HCl_{aq} (10 mL) and the reaction was stirred overnight at room temperature. The acidic solution was basified with 6N NaOH (aq) and extracted with chloroform (6×). The combined organics were dried (Na₂SO₄), filtered and concentrated by rotary evaporation to give 4-(2-(pyrrolidin-1-yl)ethoxy)butane-1,2-diol which was azeotroped with toluene (3×) and used in the next step without further purification.

4-(2-(pyrrolidin-1-yl)ethoxy)butane-1,2-diol (104 mg, 0.51 mmol) was taken up in toluene (2 mL) and NaH (49 mg, 2.05 mmol) was added carefully. The slurry was stirred for 1 hour and (9Z,12Z)-octadeca-9,12-dienyl methanesulfonate (405 mg, 1.18 mmol) was added and the reaction was heated at 85-90 C. for 4 hours, cooled and quenched carefully with ethanol and then water. More water was added along with ethyl acetate and the layers were separated. The aqueous layer was extracted with ethyl acetate (3×) and the combined organics were dried (Na₂SO₄), filtered and concentrated by rotary evaporation. The residue was purified by regular phase chromatography (Analogix 0-100% hexanes:ethyl acetate) to give Example 31. MS (ESI) m/z 700.6 (M+H); 1H NMR (300 MHz, CHLOROFORM-D) δ ppm 5.25-5.45 (m, 8H) 3.48-3.66 (m, 6H) 3.36-3.47 (m, 5H) 2.77 (t, J=5.95 Hz, 4H) 2.68 (t, J=6.15 Hz, 2H) 2.51-2.61 (m, 4H) 2.05 (q, J=6.48 Hz, 8H) 1.70-1.87 (m, 6H) 1.48-1.61 (m, 4H) 1.23-1.41 (m, 32H) 0.84-0.93 (m, 6H).

### Example 32

### N,N-diethyl-N-(2-{2-[(8Z,11Z)-heptadeca-8,11-dienyl]-2-[(9Z,12Z)-octadeca-9,12-dienyl]-1,3-dioxolan-4-yl}ethyl)amine

Example 30A (25 mg, 0.15 mmol) and (5Z,8Z,26Z,29Z)-pentatriaconta-5,8,26,29-tetraen-18-one (93 mg, 0.19 mmol) were combined in toluene (3 mL). p-Toluenesulfonic acid (2.95 mg, 0.016 mmol) was added, the flask was fitted with a small Dean-Stark trap and the reaction was refluxed overnight. Triethylamine, ethyl acetate and water were added and the layers were separated. The aqueous layer was extracted with ethyl acetate (2×) and the combined organics were dried (Na₂SO₄), filtered and concentrated by rotary evaporation. The residue was purified by regular phase chromatography (Analogix, 0-100% hexanes:ethyl acetate) to give Example 32. MS (ESI) m/z 656.6 (M+H); ¹H NMR (300 MHz, CHLOROFORM-D) δ ppm 5.26-5.46 (m, 8H) 3.99-4.14 (m, 2H) 3.40-3.54 (m, 1H) 2.77 (t, J=5.93 Hz, 4H) 2.39-2.61 (m, 6H) 2.05 (q, J=6.44 Hz, 8H) 1.63-1.86 (m, 2H) 1.19-1.43 (m, 38H) 1.02 (t, J=7.12 Hz, 6H) 0.82-0.96 (m, 6H).

### Example 33

### 1-{[(9Z)-octadec-9-enoyloxy]methyl}-3-pyrrolidin-1-ylpropyl (9Z)-octadec-9-enoate

### Example 33A

### 1-(2-(2,2-dimethyl-1,3-dioxolan-4-yl)ethyl)pyrrolidine

Example 33A was prepared using the procedure from Example 30A, substituting pyrrolidine for diethylamine. MS (ESI) m/z 199.9 (M+H); ¹H NMR (300 MHz, CHLOROFORM-D) δ ppm 4.02-4.20 (m, 2H) 3.49-3.59 (m, 1H) 2.39-2.65 (m, 6H) 1.66-1.94 (m, 6H) 1.41 (s, 3H) 1.36 (s, 3H).

### Example 33B

### 4-(pyrrolidin-1-yl)butane-1,2-diol

Example 33B was prepared from Example 33A using the procedure from Example 30B. ¹H NMR (300 MHz, CHLOROFORM-D) δ ppm 3.84-3.96 (m, 1H) 3.56-3.65 (m, 1H) 3.46-3.56 (m, 1H) 2.88-3.01 (m, 1H) 2.59-2.72 (m, 1H) 2.45-2.59 (m, 4H) 1.70-1.85 (m, 6H).

### Example 33C

### 1-{[(9Z)-octadec-9-enoyloxy]methyl}-3-pyrrolidin-1-ylpropyl (9Z)-octadec-9-enoate

Example 33B (300 mg, 1.9 mmol) was dissolved in dichloromethane (9.4 ml). Oleic acid (1.17 g, 4.15 mmol) was added followed by 1-ethyl-3-[3-(dimethylamino)propyl]-carbodiimide hydrochloride (903 mg, 4.71 mmol) and 4-dimethylaminopyridine (46 mg, 0.38 mmol). The reaction was stirred overnight at room temperature. The reaction was quenched with water and diluted with ethyl acetate. The aqueous layer was extracted with ethyl acetate (3×) and the combined organics were dried (Na₂SO₄), filtered and concentrated by rotary evaporation. The residue was purified by regular phase chromatography (Analogix, 0-100%, hexanes:ethyl acetate) to give Example 33. MS (ESI) m/z 688.6 (M+H); ¹H NMR (300 MHz, CHLOROFORM-D) δ ppm 5.27-5.42 (m, 4H) 5.09-5.19 (m, 1H) 4.24 (dd, J=11.90, 3.57 Hz, 1H) 4.06 (dd, J=11.70, 6.54 Hz, 1H) 2.44-2.60 (m, 6H) 2.23-2.35 (m, 4H) 1.94-2.08 (m, 8H) 1.74-1.86 (m, 6H) 1.54-1.67 (m, 4H) 1.28 (d, J=8.72 Hz, 40H) 0.83-0.94 (m, 6H).

### Example 34

### 1-{3,4-bis[(9Z)-octadec-9-enyloxy]butyl}pyrrolidine

Example 33B (400 mg, 2.51 mmol) was dissolved in toluene (7 mL). Sodium hydride (90%, 482 mg, 20.1 mmol) was added portion-wise. The flask was flushed with nitrogen and stirred for 30 minutes. (Z)-octadec-9-enyl methanesulfonate (2.1 g, 6.0 mmol) was taken up in toluene (3 mL) and added dropwise to the mixture. The flask was fit with a Vigourex column and refluxed for 4 hours. The reaction was cooled, quenched with ethanol and water and diluted with ethyl acetate. The layers were seperated and the aqueous layer was extracted with ethyl acetate (3×). The combined organics was dried (Na₂SO₄), filtered and concentrated by rotary evaporation. The residue was purified by regular phase chromatography (Analogix, 0-100% hexanes:ethyl acetate) to give Example 34. MS (ESI) m/z 660.7 (M+H); ¹H NMR (300 MHz, CHLOROFORM-D) δ ppm 5.27-5.42 (m, 4H) 3.50-3.62 (m, 1H) 3.35-3.49 (m, 6H) 2.43-2.58 (m, 6H) 1.92-2.09 (m, 8H) 1.65-1.83 (m, 6H) 1.49-1.62 (m, 4H) 1.28 (d, J=5.95 Hz, 44H) 0.83-0.94 (m, 6H).

### Example 35

### 1-{[(5Z,8Z,11Z,14Z)-icosa-5,8,11,14-tetraenoyloxy]methyl}-3-pyrrolidin-1-ylpropyl(5Z,8Z,11Z,14Z)-icosa-5,8,11,14-tetraenoate

Example 35 was prepared using the same procedure as Example 33, substituting (5Z,8Z,11Z,14Z)-icosa-5,8,11,14-tetraenoic acid for oleic acid. MS (ESI) m/z 732.6 (M+H); ¹H NMR (300 MHz, CHLOROFORM-D) δ ppm 5.27-5.47 (m, 16H) 5.09-5.22 (m, 1H) 4.26 (dd, J=11.90, 3.57 Hz, 1H) 4.07 (dd, J=11.90, 6.35 Hz, 1H) 2.75-2.90 (m, 12H) 2.44-2.60 (m, 6H) 2.27-2.38 (m, 4H) 1.99-2.17 (m, 8H) 1.74-1.89 (m, 6H) 1.62-1.74 (m, 4H) 1.21-1.45 (m, 12H) 0.80-0.97 (m, 6H).

### Example 36

### (3S)-3,4-bis[(9Z,12Z)-octadeca-9,12-dienyloxy]butyl 3-pyrrolidin-1-ylpropylcarbamate

### Example 36A

### (S)-4-(2-(methoxymethoxy)ethyl)-2,2-dimethyl-1,3-dioxolane

To a stirred solution of (S)-2-(2,2-dimethyl-1,3-dioxolan-4-yl)ethanol (4.2 g, 29 mmol) in CH₂Cl₂ (144 ml) was added Hunig's base (15.1 ml, 87 mmol). The reaction was cooled to 0° C. and chloro(methoxy)methane (3.1 ml, 40.4 mmol) was added dropwise. The reaction was warmed to room temperature and stirred overnight. The solution was diluted with ether and poured into saturated NH₄Cl_{(aq)}. The aqueous layer was extracted with diethyl ether (2×) and the combined organics were dried (Na₂SO₄), filtered and concentrated by rotary evaporation. The residue was purified by regular phase chromatography (Analogix, 0-100%, hexanes:ethyl acetate) to give Example 36A. MS (ESI) m/z 191.1 (M+H); ¹H NMR (300 MHz, CHLOROFORM-D) δ ppm 4.58-4.64 (m, 2H) 4.13-4.28 (m, 1H) 4.02-4.14 (m, 1H) 3.53-3.69 (m, 3H) 3.36 (s, 3H) 1.76-1.98 (m, 2H) 1.41 (s, 3H) 1.36 (s, 3H).

### Example 36B

### (S)-4-(methoxymethoxy)butane-1,2-diol

Example 36A was taken up in water (29 ml) and acetic acid (44 ml) and stirred for 2 hours at room temperature, monitoring very carefully by TLC to avoid deprotection of the MOM (methoxymethyl) group. When complete, a minimal amount of 20% NaOH was added and the basified solution was extracted with ethyl acetate (6×). The combined organics were dried (Na₂SO₄), filtered and concentrated by rotary evaporation. The residue was azeotroped with toluene (3×) and the crude material was used in the next step without further purification. ¹H NMR (300 MHz, CHLOROFORM-D) δ ppm 4.61-4.66 (m, 2H) 3.88-3.98 (m, 1H) 3.71-3.79 (m, 2H) 3.63-3.70 (m, 1H) 3.48-3.57 (m, 1H) 3.36-3.40 (m, 3H) 1.68-1.86 (m, 2H).

### Example 36C

### (6Z,9Z)-18-((S)-4-(methoxymethoxy)-1-((9Z,12Z)-octadeca-9,12-dienyloxy)butan-2-yloxy)octadeca-6,9-diene

(S)-4-(methoxymethoxy)butane-1,2-diol (200 mg, 1.33 mmol) was dissolved in toluene (5 ml). Sodium hydride (192 mg, 8.0 mmol) was added portion-wise and the mixture was stirred for 30 minutes. (9Z,12Z)-octadeca-9,12-dienyl methanesulfonate was added dropwise, the flask was fitted with a Vigourex column and the reaction was heated to 90° C. for 4 hours. The reaction was cooled to room temperature, quenched with ethanol and then water, diluted with ethyl acetate and separated. The aqueous layer was extracted with ethyl acetate (3×) and the combined organics were dried (Na₂SO₄), filtered and concentrated by rotary evaporation. The residue was purified by regular phase chromatography (Analogix, 0-100%, hexanes:ethyl acetate) to give the desired compound. MS (ESI) m/z 664.6 (M+NH₄); ¹H NMR (300 MHz, CHLOROFORM-D) δ ppm 5.25-5.46 (m, 8H) 4.62 (s, 2H) 3.59-3.70 (m, 2H) 3.50-3.59 (m, 1H) 3.38-3.47 (m, 6H) 3.36 (s, 3H) 2.77 (t, J=5.93 Hz, 4H) 1.98-2.10 (m, 8H) 1.69-1.87 (m, 2H) 1.49-1.60 (m, 4H) 1.23-1.42 (m, 32H) 0.82-0.94 (m, 6H).

### Example 36D

### (S)-3,4-bis((9Z,12Z)-octadeca-9,12-dienyloxy)butan-1-ol

Example 36C (1.84 g, 2.84 mmol) was dissolved in tetrahydrofuran (10.6 ml) and methanol (3.55 ml). To this solution, concentrated HCl_{(aq)} (5 ml) was added. The reaction was heated to 62° C. for 5 hours, cooled to room temperature and 6 N NaOH was added until the solution was basic. The aqueous layer was extracted with ethyl acetate (3×) and the combined organics were dried (Na₂SO₄), filtered and concentrated by rotary evaporation. The residue was purified by regular phase chromatography (Analogix, 0-65%, hexanes:ethyl acetate). MS (ESI) m/z 620.6 (M+NH₄); ¹H NMR (300 MHz, CHLOROFORM-D) δ ppm 5.21-5.47 (m, 8H) 3.77 (q, J=5.65 Hz, 2H) 3.55-3.70 (m, 2H) 3.36-3.56 (m, 6H) 2.71-2.83 (m, 4H) 1.98-2.10 (m, 8H) 1.74-1.87 (m, 2H) 1.58 (s, 4H) 1.22-1.44 (m, 32H) 0.82-0.96 (m, 6H).

### Example 36E

### (S)-3,4-bis((9Z,12Z)-octadeca-9,12-dienyloxy)butyl 4-nitrophenyl carbonate

Example 36D (27 mg, 0.045 mmol) was taken up in dichloromethane (1 ml) and triethylamine (6.2 uL) was added followed by 4-nitrophenyl carbonochloridate (11 mg, 0.054 mmol). The reaction was allowed to stir overnight. The reaction mixture was loaded directly onto a silica gel column and purified (Analogix, 0-75%, hexanes:ethyl acetate) to provide Example 36E. ¹H NMR (300 MHz, CHLOROFORM-D) δ ppm 8.28 (d, J=9.16 Hz, 2H) 7.38 (d, J=9.16 Hz, 2H) 5.27-5.44 (m, 8H) 4.43 (dd, J=7.46, 5.76 Hz, 2H) 3.37-3.71 (m, 8H) 2.77 (t, J=6.10 Hz, 4H) 1.97-2.11 (m, 8H) 1.92 (s, 2H) 1.56-1.63 (m, 4H) 1.19-1.45 (m, 32H) 0.80-0.97 (m, 6H).

### Example 36F

### (3S)-3,4-bis[(9Z,12Z)-octadeca-9,12-dienyloxy]butyl 3-pyrrolidin-1-ylpropylcarbamate

Example 36E (34.4 mg, 0.045 mmol) and 3-(pyrrolidin-1-yl)propan-1-amine (excess) were combined in dichloromethane (1 ml) and stirred overnight. The mixture was loaded directly onto silica gel for purification (Analogix, 0-100%, hexanes:ethyl acetate) to give Example 36. MS (ESI) m/z 757.7 (M+H); ¹H NMR (300 MHz, CHLOROFORM-D) δ ppm 5.56 (s, 1H) 5.26-5.45 (m, 8H) 4.11-4.22 (m, 2H) 3.52-3.65 (m, 1H) 3.35-3.52 (m, 6H) 3.26 (q, J=5.88 Hz, 2H) 2.72-2.84 (m, 4H) 2.49-2.62 (m, 6H) 2.05 (q, J=6.56 Hz, 8H) 1.66-1.92 (m, 8H) 1.47-1.63 (m, 4H) 1.19-1.42 (m, 32H) 0.82-0.96 (m, 6H).

### Example 37

### 1-[3,4-bis(octadecyloxy)butyl]pyrrolidine

Example 37 was prepared using the same procedure described in Example 1B substituting pyrrolidine for piperidine and octadecyl methanesulfonate for (9Z,12Z)-octadeca-9,12-dienyl methanesulfonate. MS (ESI) m/z 664.7 (M+H); ¹H NMR (300 MHz, CHLOROFORM-D) δ ppm 3.51-3.62 (m, 1H) 3.36-3.51 (m, 6H) 2.45-2.57 (m, 6H) 1.66-1.82 (m, 6H) 1.48-1.60 (m, 4H) 1.16-1.37 (m, 60H) 0.83-0.93 (m, 6H).

### Example 38

### 1-[3,4-bis(hexadecyloxy)butyl]pyrrolidine

Example 38 was prepared using the same procedure described in Example 1B, substituting pyrrolidine for piperidine and hexyldecyl methanesulfonate for (9Z,12Z)-octadeca-9,12-dienyl methanesulfonate. MS (ESI) m/z 664.7 (M+H); H NMR (300 MHz, CHLOROFORM-D) δ ppm 3.51-3.64 (m, 1H) 3.36-3.50 (m, 6H) 2.41-2.60 (m, 6H) 1.66-1.81 (m, 6H) 1.47-1.59 (m, 4H) 1.26 (s, 52H) 0.88 (t, J=6.44 Hz, 6H).

### Example 39

### 1-{3,4-bis[(9E)-hexadec-9-enyloxy]butyl}pyrrolidine

Example 39 was prepared using the same procedure described in Example 1B substituting pyrrolidine for piperidine and (E)-hexadec-9-enyl methanesulfonate for (9Z,12Z)-octadeca-9,12-dienyl methanesulfonate. MS (ESI) m/z 604.7 (M+H); ¹H NMR (300 MHz, CHLOROFORM-D) δ ppm 5.30-5.46 (m, 4H) 3.51-3.62 (m, 1H) 3.33-3.51 (m, 6H) 2.43-2.58 (m, 6H) 1.89-2.03 (m, J=4.76 Hz, 8H) 1.62-1.83 (m, 6H) 1.46-1.61 (m, 4H) 1.20-1.41 (m, 36H) 0.82-0.95 (m, 6H).

### Example 40

### 1-{3,4-bis[(9E)-octadec-9-enyloxy]butyl}pyrrolidine

Example 40 was prepared using the same procedure described in Example 1B substituting pyrrolidine for piperidine and (E)-octadec-9-enyl methanesulfonate for (9Z,12Z)-octadeca-9,12-dienyl methanesulfonate. MS (ESI) m/z 660.7 (M+H); ¹H NMR (300 MHz, CHLOROFORM-D) δ ppm 5.30-5.46 (m, 4H) 3.51-3.64 (m, 1H) 3.35-3.50 (m, 6H) 2.42-2.58 (m, 6H) 1.89-2.04 (m, 8H) 1.65-1.83 (m, 6H) 1.47-1.57 (m, 4H) 1.17-1.40 (m, 44H) 0.81-0.94 (m, 6H).

### Example 41

### 1-{3,4-bis[(9E,12E)-octadeca-9,12-dienyloxy]butyl}pyrrolidine

Example 41 was prepared using the same procedure described in Example 1B substituting pyrrolidine for piperidine and (9E,12E)-octadeca-9,12-dienyl methanesulfonate for (9Z,12Z)-octadeca-9,12-dienyl methanesulfonate. MS (ESI) m/z 656.7 (M+H); ¹H NMR (300 MHz, CHLOROFORM-D) δ ppm 5.31-5.50 (m, 8H) 3.51-3.63 (m, 1H) 3.34-3.52 (m, 6H) 2.62-2.73 (m, 4H) 2.42-2.59 (m, 6H) 1.91-2.04 (m, 8H) 1.67-1.81 (m, 6H) 1.48-1.58 (m, 4H) 1.18-1.41 (m, 32H) 0.78-0.94 (m, 6H).

### Example 42

### 1-{3,4-bis[(9Z,12Z,15Z)-octadeca-9,12,15-trienyloxy]butyl}pyrrolidine

Example 42 was prepared using the same procedure described in Example 1B substituting pyrrolidine for piperidine and (9Z,12Z,15Z)-octadeca-9,12,15-trienyl methanesulfonate for (9Z,12Z)-octadeca-9,12-dienyl methanesulfonate. LCMS m/z 652.8 (M+H); ¹H NMR (300 MHz, CHLOROFORM-D) δ ppm 5.24-5.48 (m, 12H) 3.50-3.63 (m, 1H) 3.35-3.51 (m, 6H) 2.81 (t, J=5.76 Hz, 8H) 2.42-2.59 (m, 6H) 1.99-2.20 (m, 8H) 1.66-1.83 (m, 6H) 1.55 (s, 4H) 1.23-1.41 (m, 20H) 0.87-1.07 (m, 6H).

### Example 43

### N¹-{(3S)-3,4-bis[(9Z,12Z)-octadeca-9,12-dienyloxy]butyl}-N³,N³-diethyl-beta-alaninamide

### Example 43A

### (6Z,9Z)-18-((S)-4-azido-1-((9Z,12Z)-octadeca-9,12-dienyloxy)butan-2-yloxy)octadeca-6,9-diene

To a solution of Example 36D (40 mg, 0.066 mmol) in tetrahydrofuran (about 1 ml) was added triphenylphosphine (87 mg, 0.332 mmol), diethyl azodicarboxylate (52.5 uL, 0.332 mmol) and diphenylphosphoryl azide (68.8 ul, 0.318 mmol). The reaction was stirred at room temperature for about 30 minutes, passed through a plug of silica gel and then chromatographed (Analogix, 0-25%, hexanes:ethyl acetate) to give Example 43A. ¹H NMR (300 MHz, CHLOROFORM-D) δ ppm 5.25-5.46 (m, 8H) 3.62 (d, J=9.12 Hz, 1H) 3.34-3.55 (m, 7H) 2.77 (t, J=6.15 Hz, 4H) 2.05 (q, J=6.61 Hz, 8H) 1.79 (d, J=4.36 Hz, 2H) 1.58 (s, 4H) 1.18-1.41 (m, 34H) 0.79-0.96 (m, 6H).

### Example 43B

### (S)-3,4-bis((9Z,12Z)-octadeca-9,12-dienyloxy)butan-1-amine

Example 43A (20 mg, 0.032 mmol) was taken up in tetrahydrofuran (0.5 ml) and water (0.5 ml). PPh₃ (17 mg, 0.064 mmol) was added and the reaction was heated to reflux. The reaction was cooled and extracted with ethyl acetate (5×) and the combined organics were dried (Na₂SO₄), filtered and concentrated by rotary evaporation. The crude material was used in the next step without further purification. LCMS m/z 603.8 (M+H).

### Example 43C

### N¹-{(3S)-3,4-bis[(9Z,12Z)-octadeca-9,12-dienyloxy]butyl}-N³,N³-diethyl-beta-alaninamide

(S)-3,4-bis((9Z,12Z)-octadeca-9,12-dienyloxy)butan-1-amine (19 mg, 0.032 mmol) and 3-(diethylamino)propanoic acid (9.2 mg, 0.063 mmol) were dissolved in dichloromethane (0.5 mL) at room temperature. 1-Ethyl-3-[3-(dimethylamino)propyl]-carbodiimide hydrochloride (12.1 mg, 0.63 mmol) and 4-dimethylaminopyridine (3.9 mg, 0.032 mmol) were added sequentially and the reaction was stirred overnight at room temperature. The reaction was loaded directly onto silica gel and purified by regular phase chromatography (Analogix, 0-100%, hexanes:ethyl acetate). MS (ESI) m/z 730.7 (M+H).

### Example 44

### N-{3,4-bis[(9Z,12Z)-octadeca-9,12-dienyloxy]butyl}-N-[3-(1H-imidazol-1-yl)propyl]amine

### Example 44A

### 4-(2-(methoxymethoxy)ethyl)-2,2-dimethyl-1,3-dioxolane

Example 44A was prepared using the same procedure described for Example 36A, substituting 2-(2,2-dimethyl-1,3-dioxolan-4-yl)ethanol for (S)-2-(2,2-dimethyl-1,3-dioxolan-4-yl)ethanol. ¹H NMR (300 MHz, CHLOROFORM-D) δ ppm 4.61 (s, 2H) 4.21 (t, J=6.54 Hz, 1H) 4.03-4.14 (m, 2H) 3.53-3.68 (m, 2H) 3.34-3.39 (m, 3H) 1.76-1.98 (m,2H) 1.41 (s, 3H) 1.36 (s, 3H).

### Example 44B

### 4-(methoxymethoxy)butane-1,2-diol

Example 44B was prepared using the same procedure described for 36B, substituting Example 44A for Example 36A and used without further purification.

### Example 44C

### (6Z,9Z)-18-(4-(methoxymethoxy)-1-((9Z,12Z)-octadeca-9,12-dienyloxy)butan-2-yloxy)octadeca-6,9-diene

Example 44C was prepared using the same procedure described for 36C, substituting Example 44B for Example 36B. ¹H NMR (300 MHz, CHLOROFORM-D) δ ppm 5.25-5.45 (m, 8H) 4.62 (s, 2H) 3.48-3.70 (m, 3H) 3.38-3.48 (m, 6H) 3.36 (s, 3H) 2.77 (t, J=6.15 Hz, 4H) 2.05 (q, J=6.74 Hz, 8H) 1.66-1.91 (m, 2H) 1.54 (s, 4H) 1.20-1.43 (m, 32H) 0.84-0.93 (m, 6H).

### Example 44D

### 3,4-bis((9Z,12Z)-octadeca-9,12-dienyloxy)butan-1-ol

Example 44D was prepared using the same procedure described for 36D, substituting Example 44C for Example 36C. (ESI) m/z 620.7 (M+NH₄); ¹H NMR (300 MHz, CHLOROFORM-D) δ ppm 5.26-5.45 (m, 8H) 3.77 (q, J=5.43 Hz, 2H) 3.59-3.68 (m, 1H) 3.38-3.56 (m, 6H) 2.77 (t, J=5.59 Hz, 4H) 2.05 (q, J=6.56 Hz, 8H) 1.74-1.86 (m, 2H) 1.55-1.61 (m, 4H) 1.22-1.43 (m, 32H) 0.85-0.95 (m, 6H).

### Example 44E

### 3,4-bis((9Z,12Z)-octadeca-9,12-dienyloxy)butanal

3,4-bis((9Z, 12Z)-octadeca-9,12-dienyloxy)butan-1-ol (1 g, 1.7 mmol) was dissolved in dichloromethane (17 ml) at room temperature and Dess-Martin Periodinane (2.8 g, 6.6 mmol) was added as a solid. The mixture was stirred overnight and sodium thiosulfate (saturated, aqueous) was added and the mixture was stirred for 1 hour. More dichloromethane and water were added and the layers were separated. The aqueous layer was extracted with dichloromethane (3×) and the combined organics were dried (MgSO₄), filtered and concentrated by rotary evaporation. The residue was used in the next step without further purification.

### Example 44F

### N-{3,4-bis[(9Z,12Z)-octadeca-9,12-dienyloxy]butyl}-N-[3-(1H-imidazol-1-yl)propyl]amine

3,4-bis((9Z,12Z)-octadeca-9,12-dienyloxy)butanal (100 mg, 0.17 mmol) was dissolved in a buffer solution (2 ml) consisting of sodium acetate, acetic acid and methanol (stock solution premixed: 6 g sodium acetate, 8.5 ml acetic acid in 250 ml methanol). 3-(1H-imidazol-1-yl)propan-1-amine (31.2 mg, 0.250 mmol) was added followed by sodium triacetoxyborohydride (106 mg, 0.50 mmol). The reaction was stirred overnight and loaded directly onto silica gel for purification (Analogix, 0-100%, hexanes:ethyl acetate) to provide Example 44. MS (ESI) m/z 710.6 (M+H); ¹H NMR (300 MHz, CHLOROFORM-D) δ ppm 7.53 (s, 1H) 7.07 (s, 1H) 6.92 (s, 1H) 5.25-5.46 (m, 8H) 4.04 (t, J=6.94 Hz, 2H) 3.53-3.67 (m, 1H) 3.34-3.53 (m, 6H) 2.77 (t, J=6.15 Hz, 6H) 2.65 (t, J=6.94 Hz, 2H) 1.92-2.10 (m, 10H) 1.64-1.88 (m, 2H) 1.48-1.61 (m, 4H) 1.21-1.42 (m, 32H) 0.79-0.96 (m, 6H).

### Example 45

### N-{3,4-bis[(9Z,12Z)-octadeca-9,12-dienyloxy]butyl}-N,N′,N′-trimethylpropane-1,3-diamine

Example 45 was prepared using the procedure described for Example 44, substituting N¹,N¹,N³-trimethylpropane-1,3-diamine for 3-(1H-imidazol-1-yl)propan-1-amine. MS (ESI) m/z 701.6 (M+H); ¹H NMR (300 MHz, CHLOROFORM-D) δ ppm 5.24-5.47 (m, 8H) 3.51-3.64 (m, 1H) 3.34-3.49 (m, 6H) 2.77 (t, J=5.95 Hz, 4H) 2.36-2.58 (m, 6H) 2.27-2.31 (m, 6H) 2.25 (s, 3H) 1.99-2.10 (m, 8H) 1.61-1.78 (m, 4H) 1.49-1.61 (m, 4H) 1.21-1.43 (m, 32H) 0.84-0.94 (m, 6H).

### Example 46

### 1-(1-{3,4-bis[(9Z,12Z)-octadeca-9,12-dienyloxy]butyl}pyrrolidin-3-yl)-1H-imidazole

Example 46 was prepared using the procedure described for Example 44, substituting 1-(pyrrolidin-3-yl)-1H-imidazole for 3-(1H-imidazol-1-yl)propan-1-amine. MS (ESI) m/z 722.6 (M+H); ¹H NMR (300 MHz, CHLOROFORM-D) δ ppm 7.54-7.68 (m, 1H) 7.07-7.14 (m, 1H) 7.04 (s, 1H) 5.25-5.45 (m, 8H) 4.58-4.71 (m, 1H) 3.54-3.69 (m, 1H) 3.33-3.54 (m, 6H) 2.80-3.07 (m, 2H) 2.71-2.82 (m, 4H) 2.36-2.72 (m, 4H) 1.99-2.11 (m, 8H) 1.64-2.01 (m, 4H) 1.48-1.63 (m, 4H) 1.22-1.42 (m, 32H) 0.83-0.94 (m, 6H).

### Example 47

### N-{3,4-bis[(9Z,12Z)-octadeca-9,12-dienyloxy]butyl}-N-(3-pyrrolidin-1-ylpropyl)amine

Example 47 was prepared using the procedure described for Example 44, substituting 3-(pyrrolidin-1-yl)propan-1-amine for 3-(1H-imidazol-1-yl)propan-1-amine. MS (ESI) m/z 713.7 (M+H); ¹H NMR (300 MHz, CHLOROFORM-D) δ ppm 5.25-5.45 (m, 8H) 3.53-3.64 (m, 1H) 3.34-3.50 (m, 6H) 2.84-2.96 (m, 4H) 2.69-2.82 (m, 6H) 2.00-2.12 (m, 10H) 1.77-1.93 (m, 8H) 1.47-1.62 (m, 4H) 1.22-1.41 (m, 32H) 0.81-0.98 (m, 6H).

### Example 48

### N-{3,4-bis[(9Z,12Z)-octadeca-9,12-dienyloxy]butyl}-N′,N′-dimethylpropane-1,3-diamine

Example 48 was prepared using the procedure described for Example 44, substituting N¹,N¹-dimethylpropane-1,3-diamine for 3-(1H-imidazol-1-yl)propan-1-amine. MS (ESI) m/z 687.7 (M+H); ¹H NMR (300 MHz, CHLOROFORM-D) δ ppm 5.19-5.46 (m, 8H) 3.36-3.65 (m, 7H) 2.90-3.03 (m, 4H) 2.77 (t, J=5.93 Hz, 4H) 2.58 (t, J=6.61 Hz, 2H) 2.33 (s, 6H) 1.98-2.11 (m, 8H) 1.71-1.96 (m, 4H) 1.45-1.61 (m, 4H) 1.22-1.43 (m, 32H) 0.82-0.95 (m, 6H).

### Example 49

### 1-{3,4-bis[(9Z,12Z)-octadeca-9,12-dienyloxy]butyl}azetidine

Example 49 was prepared using the procedure described for Example 44, substituting azetidine for 3-(1H-imidazol-1-yl)propan-1-amine. MS (ESI) m/z 642.6 (M+H); ¹H NMR (300 MHz, CHLOROFORM-D) δ ppm 5.24-5.47 (m, 8H) 3.50-3.65 (m, 1H) 3.32-3.52 (m, 6H) 2.77 (t, J=5.93 Hz, 4H) 2.57-2.70 (m, 2H) 2.12-2.25 (m, 2H) 1.98-2.11 (m, 12H) 1.47-1.71 (m, 6H) 1.21-1.44 (m, 32H) 0.82-0.95 (m, 6H).

### Example 50

### 1-{3,4-bis[(9Z,12Z)-octadeca-9,12-dienyloxy]butyl}-2-methylpyrrolidine

Example 50 was prepared using the procedure described for Example 44, substituting 2-methylpyrrolidine for 3-(1H-imidazol-1-yl)propan-1-amine. MS (ESI) m/z 670.6 (M+H); ¹H NMR (300 MHz, CHLOROFORM-D) δ ppm 5.26-5.46 (m, 8H) 3.36-3.64 (m, 7H) 2.77 (t, J=6.10 Hz, 4H) 2.27 (s, 1H) 1.98-2.13 (m, 11H) 1.66-1.98 (m, 6H) 1.55 (s, 4H) 1.21-1.44 (m, 32H) 1.09 (t, J=6.10 Hz, 3H) 0.82-0.95 (m, 6H).

### Example 51

### 1-{3,4-bis[(9Z,12Z)-octadeca-9,12-dienyloxy]butyl}-2,5-dimethylpyrrolidine

Example 51 was prepared using the procedure described for Example 44, substituting 2,5-dimethylpyrrolidine for 3-(1H-imidazol-1-yl)propan-1-amine. MS (ESI) m/z 684.6 (M+H); ¹H NMR (300 MHz, CHLOROFORM-D) δ ppm 5.26-5.47 (m, 8H) 3.53-3.67 (m, 1H) 3.33-3.49 (m, 6H) 3.16 (s, 4H) 2.69-2.88 (m, 6H) 1.97-2.13 (m, 8H) 1.61-1.92 (m, 4H) 1.47-1.61 (m, 4H) 1.23-1.42 (m, 32H) 1.12-1.23 (m, 6H) 0.80-0.95 (m, 6H).

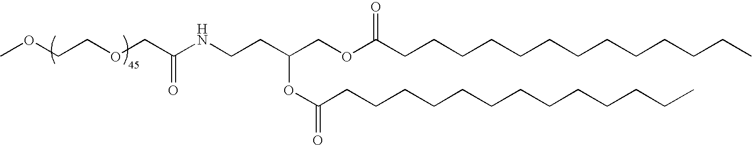

### Example 52

### 6-oxo-2-(tetradecanoyloxy)-8,11,14,17,20,23,26,29,32,35,38,41,44,47,50,53,56,59,62,65,68,71,74,77,80,83,86,89,92,95,9 8,101,104,107,110,113,116,119,122,125,128,131,134,137,140,143-hexatetracontaoxa-5-azatetratetracontahect-1-yl myristate

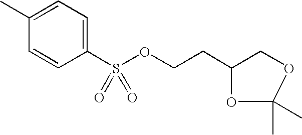

### Example 52A

### 2-(2,2-dimethyl-1,3-dioxolan-4-yl)ethyl 4-methylbenzenesulfonate

2-(2,2-Dimethyl-1,3-dioxolan-4-yl)ethanol (5 g) was added to dichloromethane (86 ml) and the mixture was cooled to 0° C. To this solution was added triethylamine (6.9 g, 9.6 ml), tosyl chloride (6.5 g) and 4-(dimethylamino)pyridine (0.42 g). The mixture stirred at room temperature overnight. The mixture was quenched with saturated NH₄Cl and diluted with ethyl acetate. The aqueous layer was extracted twice with ethyl acetate and the extract was dried (Na₂SO₄), filtered, and concentrated. The concentrate was purified by flash column chromatography (Analogix hexanes:ethyl acetate, 0-75%) to afford the title compound. MS (ESI) m/z 300.9 (M+H)⁺; ¹H NMR (400 MHz, CDCl₃) δ 7.79 (d, J=8.29 Hz, 2H) 7.35 (d, J=7.98 Hz, 2H) 4.06-4.23 (m, 3H) 4.01 (dd, J=7.98, 6.14 Hz, 1H) 3.51 (dd, J=8.13, 6.90 Hz, 1H) 2.45 (s, 3H) 1.82-1.98 (m, 2H) 1.31 (d, J=18.72 Hz, 6H).

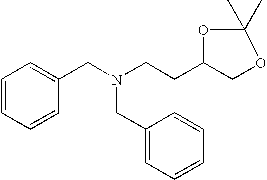

### Example 52B

### N,N-dibenzyl-2-(2,2-dimethyl-1,3-dioxolan-4-yl)ethanamine

Example 52A (1.0 g) and dibenzylamine (0.657 mg) were placed in a microwave vial (Biotage) and dioxane (2.5 mL) was added. The vial was capped and placed in a microwave reactor (Biotage Initiator), and the mixture was heated at 150° C. for 30 minutes. The mixture was diluted with ethyl acetate and poured into water. The aqueous layer was extracted twice with ethyl acetate, and the extract was washed with brine, dried (Na₂SO₄), filtered and concentrated. The concentrate was used in the next step without further purification.

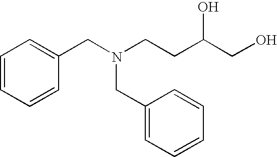

### Example 52C

### 4-(dibenzylamino)butane-1,2-diol

Example 52B was added to tetrahydrofuran (20 mL) and 2N HCl (20 mL), and the mixture was stirred at room temperature for 30 minutes. 5N NaOH was added until the solution was basic, and the aqueous layer was extracted with chloroform. The extract was dried (MgSO₄), filtered and concentrated by rotary evaporation and the concentrate was used in the next step without further purification. MS (ESI) m/z 285.9 (M+H)⁺.

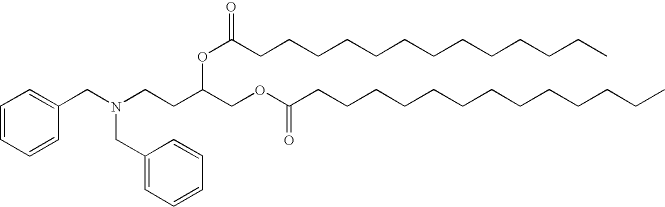

### Example 52D

### 4-(dibenzylamino)butane-1,2-diyl ditetradecanoate

A mixture of Example 52C (700 mg), tetradecanoic acid (1.68 g), N¹-((ethylimino)methylene)-N³,N³-dimethylpropane-1,3-diamine hydrochloride (1.41 g) and 4-(dimethylamino)pyridine (45 mg) in dichloromethane (5 mL) was heated at 40° C. until the mixture was homogenous and then was stirred overnight at room temperature. Water was added along with some brine and the aqueous layer was extracted with dichloromethane (3×). The extract was dried (Na₂SO₄), filtered and the filtrate was concentrated. The concentrate was purified by flash column chromatography (Analogix 280, 0-50% ethyl acetate/hexanes) to provide the title compound. MS (ESI) m/z 706.5 (M+H)⁺; ¹H NMR (300 MHz, CDCl₃) δ 7.16-7.39 (m, 10H) 5.06-5.21 (m, 1H) 4.12 (dd, J=11.70, 3.37 Hz, 1H) 3.91 (dd, J=11.90, 5.95 Hz, 1H) 3.41-3.62 (m, 4H) 2.35-2.57 (m, 2H) 2.25 (t, J=7.54 Hz, 2H) 2.02-2.19 (m, 2H) 1.77 (q, J=7.40 Hz, 2H) 1.45-1.63 (m, 4H) 1.17-1.36 (m, 40H) 0.82-0.94 (m, 6H).

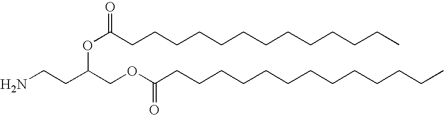

### Example 52E

### 4-aminobutane-1,2-diyl ditetradecanoate

Example 52D (500 mg) was added to methanol/dichloromethane/ethyl acetate (1/1/1, 10 mL) and combined with catalytic Pd/C (10%). Hydrogen was introduced via a balloon, and the mixture was stirred overnight then filtered through Celite®. The filtrate was concentrated and the concentrate was used in the next step without further purification. MS (ESI) m/z 526.6 (M+H)⁺; ¹H NMR (300 MHz, CDCl₃) δ ppm 5.13-5.25 (m, 1H) 4.02-4.35 (m, 2H) 2.91-3.23 (m, 2H) 2.24-2.42 (m, 4H) 1.97-2.23 (m, 2H) 1.44-1.73 (m, 6H) 1.26 (s, 40H) 0.81-0.96 (m, 6H).

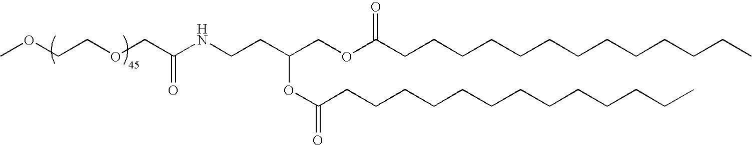

### Example 52F

### 6-oxo-2-(tetradecanoyloxy)-8,11,14,17,20,23,26,29,32,35,38,41,44,47,50,53,56,59,62,65,68,71,74,77,80,83,86,89,92,95,9 8,101,104,107,110,113,116,119,122,125,128,131,134,137,140,143-hexatetracontaoxa-5-azatetratetracontahect-1-yl myristate

mPEG2000-SCM (139 mg, Laysan Bio, Inc) and Example 52E (100 mg) were combined in a 4 mL vial with dichloromethane (1 mL) and triethylamine (26.5 μL). The mixture was stirred at room temperature overnight. The mixture mixture was loaded directly onto a silica gel column (Analogix) and eluted with dichlormethane/methanol (0-20%). MS (MALDI) m/z 2690.5; ¹H NMR (300 MHz, CDCl₃) δ ppm 5.07-5.20 (m, 1H) 4.24 (dd, J=11.90, 3.17 Hz, 1H) 4.06 (dd, J=11.90, 6.35 Hz, 1H) 3.98 (s, 2H) 3.85-3.91 (m, 1H) 3.61-3.70 (m, 29H) 3.39-3.59 (m, 6H) 3.38 (s, 3H) 3.14-3.30 (m, 1H) 2.25-2.36 (m, 4H) 1.53-1.87 (m, 6H) 1.26 (s, 40H) 0.83-0.93 (m, 6H).

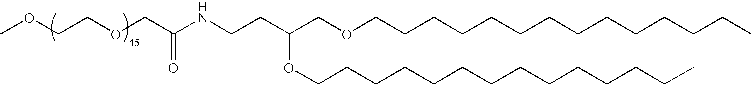

### Example 53

### N- [3,4-bis(tetradecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

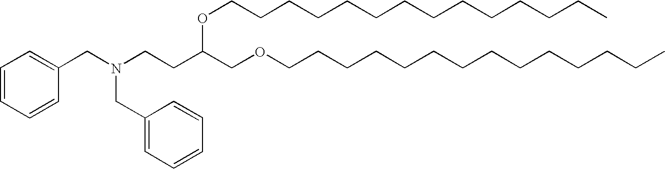

### Example 53A

### N,N-dibenzyl-3,4-bis(tetradecyloxy)butan-1-amine

Example 52C (1 g) in toluene (6 mL) and added to NaH (0.336 g, dry, 95%) in toluene (6 mL). The mixture was stirred at room temperature for 1 hour. Tetradecyl methanesulfonate (2.15 g) was added. The mixture was heated to 90° C. overnight. The mixture was cooled to room temperature and ethanol was added followed by water until the excess NaH was destroyed. The mixture was poured into water and brine and extreaced with ethyl acetate. The water was extracted with ethyl acetate, and the extract was dried (Na₂SO₄), filtered and concentrated. The concentrate was purified by an Analogix system (hexane:ethyl acetate, 0-50%)). MS (ESI) m/z 678.6 (M+H)⁺; ¹H NMR (300 MHz, CDCl₃) δ ppm 7.16-7.40 (m, 10H) 3.14-3.63 (m, 11H) 2.44-2.59 (m, 2H) 1.59-1.82 (m, 2H) 1.35-1.53 (m, 4H) 1.14-1.34 (m, 44H) 0.82-0.94 (m, 6H).

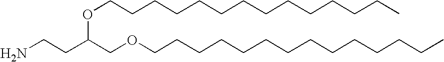

### Example 53B

### 3,4-bis(tetradecyloxy)butan-1-amine

Example 53B was prepared using the procedure described for Example 52E, substituting Example 53A for Example 52D. MS (ESI) m/z 498.5 (M+H)⁺; ¹H NMR (300 MHz, CDCl₃) δ ppm 8.24 (s, 2H) 3.53-3.70 (m, 1H) 3.34-3.53 (m, 6H) 3.07-3.34 (m, 2H) 1.87-2.13 (m, 2H) 1.48-1.67 (m, 4H) 1.16-1.39 (m, 44H) 0.82-0.94 (m, 6H).

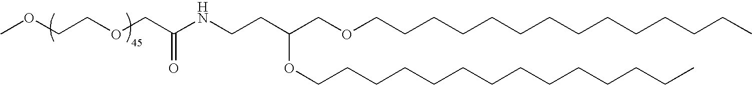

### Example 53C

### N-[3,4-bis(tetradecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

Example 53C was prepared using the procedure described for Example 52F, substituting Example 53B for Example 52E. MS (MALDI) m/z 2617.6; ¹H NMR (300 MHz, CDCl₃) δ 3.95-4.02 (m, 2H) 3.83-3.92 (m, 1H) 3.68-3.72 (m, 1H) 3.65 (m, 180H) 3.35-3.60 (m, 10H) 1.59-1.73 (m, 2H) 1.49-1.60 (m, 4H) 1.18-1.36 (m, 44H) 0.82-0.94 (m, 6H).

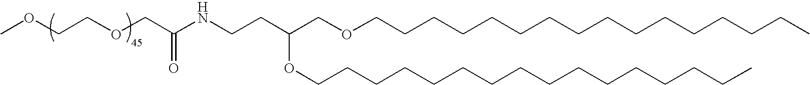

### Example 54

### N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

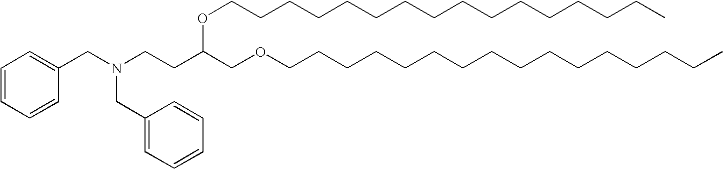

### Example 54A

### N,N-dibenzyl-3,4-bis(hexadecyloxy)butan-1-amine

Example 54A was prepared using the procedure described for Example 53A, substituting hexadecyl methanesulfonate for tetradecyl methanesulfonate. MS (ESI) m/z 734.6 (M+H)⁺; ¹H NMR (300 MHz, CDCl₃) δ ppm 7.15-7.41 (m, 10H) 3.12-3.64 (m, 11H) 2.41-2.64 (m, 2H) 1.35-1.80 (m, 6H) 1.15-1.34 (m, 52H) 0.81-0.94 (m, 6H).

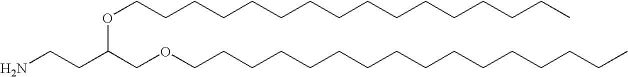

### Example 54B

### 3,4-bis(hexadecyloxy)butan-1-amine

Example 54B was prepared using the procedure described for Example 52E, substituting Example 54A for Example 52D. MS (ESI) m/z 554.6 (M+H)⁺; ¹H NMR (300 MHz, CDCl₃) δ ppm 8.12-8.38 (m, 2H) 3.54-3.70 (m, 1H) 3.33-3.53 (m, 6H) 3.06-3.33 (m, 2H) 1.84-2.14 (m, 2H) 1.46-1.71 (m, 4H) 1.14-1.37 (m, 52H) 0.81-0.94 (m, 6H).

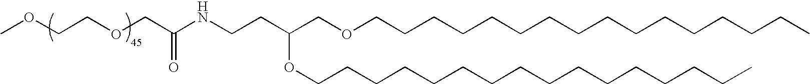

### Example 54C

### N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,1 19,122,125,128,131,134,137-hexatetracontaoxanonatriacontahectan-139-amide

Example 54C was prepared using the procedure described for Example 52F, substituting Example 54B for Example 52E. MS (MALDI) m/z 2866.7; ¹H NMR (300 MHz, CDCl₃) δ ppm 3.98 (s, 2H) 3.84-3.91 (m, 1H) 3.60-3.68 (m, 180H) 3.36-3.60 (m, 11H) 1.50-1.72 (m, 6H) 1.26 (s, 52H) 0.84-0.92 (m, 6H).

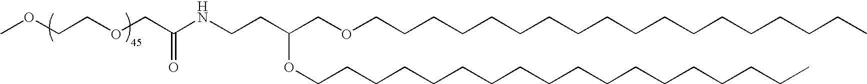

### Example 55

### N-[3,4-bis(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,1 19,122,125,128,131,134,137-hexatetracontaoxanonatriacontahectan-139-amide

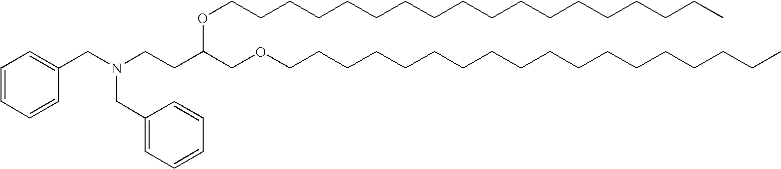

### Example 55A

### N,N-dibenzyl-3,4-bis(octadecyloxy)butan-1-amine

Example 55A was prepared using the same procedure described for Example 53A, substituting octadecyl methanesulfonate for tetradecyl methanesulfonate. LCMS (APCI) m/z 790.6; ¹H NMR (300 MHz, CDCl₃) δ ppm 7.15-7.41 (m, 10H) 3.10-3.68 (m, 11H) 2.39-2.68 (m, 2H) 1.35-1.80 (m, 6H) 1.14-1.34 (m, 60H) 0.81-0.94 (m, 6H).

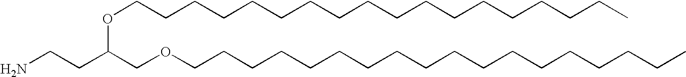

### Example 55B

### 3,4-bis(octadecyloxy)butan-1-amine

Example 55B was prepared using the same procedure described for Example 52E, substituting Example 55A for Example 52D. LCMS (APCI) m/z 610.9; ¹H NMR (300 MHz, CDCl₃) δ 3.08-3.70 (m, 9H) 1.85-2.15 (m, 2H) 1.55 (s, 4H) 1.15-1.37 (m, 60H) 0.84-0.92 (m, 6H).

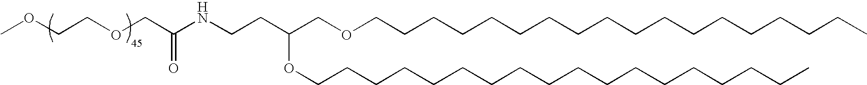

### Example 55C

### N-[3,4-bis(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,1 19,122,125,128,131,134,137-hexatetracontaoxanonatriacontahectan-139-amide

Example 55C was prepared using the same procedure described for Example 52F, substituting Example 55B for Example 52E. MS (MALDI) m/z 2773.6; ¹H NMR (300 MHz, CDCl₃) δ ppm 3.95-4.01 (m, 2H) 3.84-3.91 (m, 1H) 3.59-3.70 (m, 180H) 3.27-3.59 (m, 11H) 1.49-1.86 (m, 6H) 1.18-1.35 (m, 60H) 0.80-0.94 (m, 6H).

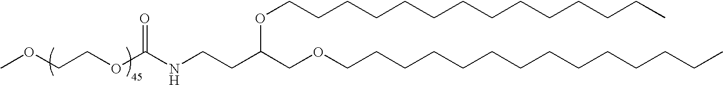

### Example 56

### 3,7,11,15,19,23,27,31,35,39,43,47,51,55,59,63,67,71,75,79,83,87,91,95,99,103,107,111,115, 119,123,127,131,135,139,143,147,151,155,159,163,167,171,175,179,182-hexatetracontaoxatrioctacontahect-1-yl 3,4-bis(tetradecyloxy)butylcarbamate

Example 53B (100 mg) was dissolved in dichloromethane (1-2 mL) and mPEG-NPC (26.0 mg) was added. Hunig's base (26 mg) was added, and the mixture was stirred overnight at room temperature. The mixture was loaded directly onto a silica gel column (4 g Analogix) and chromatographed (Analogix 280, dichloromethane/methanol, 0-20%) to give Example 56. MS (MALDI) m/z 2472.2; ¹H NMR (300 MHz, CDCl₃) δ ppm 4.16-4.24 (m, 2H) 3.78-3.92 (m, 1H) 3.59-3.70 (m, 180H) 3.52-3.61 (m, 4H) 3.19-3.49 (m, 9H) 1.48-1.82 (m, 6H) 1.21-1.35 (m, 44H) 0.82-0.93 (m, 6H).

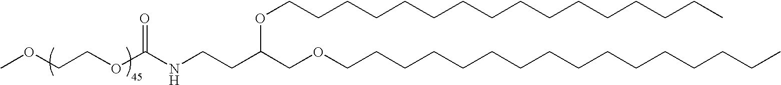

### Example 57

### 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-yl 3,4-bis(hexadecyloxy)butylcarbamate

Example 57 was prepared using the same procedure described for Example 56, substituting Example 54B for Example 53B. MS (MALDI) m/z 2395.0; ¹H NMR (300 MHz, CDCl₃) δ ppm 4.15-4.23 (m, 2H) 3.81-3.92 (m, 1H) 3.60-3.71 (m, 180H) 3.47-3.59 (m, 4H) 3.33-3.48 (m, 9H) 1.48-1.81 (m, 6H) 1.19-1.34 (m, 52H) 0.83-0.92 (m, 6H).

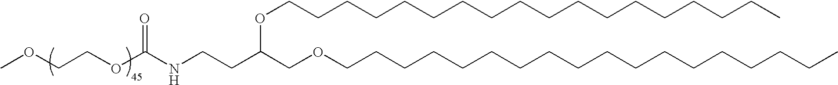

### Example 58

### 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-yl 3,4-bis(octadecyloxy)butylcarbamate

Example 58 was prepared using the same procedure described for Example 56, substituting Example 55B for Example 53B. MS (MALDI) m/z 2495.8; ¹H NMR (300 MHz, CDCl₃) δ ppm 4.16-4.24 (m, 2H) 3.82-3.92 (m, 1H) 3.60-3.71 (m, 180H) 3.49-3.59 (m, 4H) 3.17-3.49 (m, 9H) 1.48-1.80 (m, 6H) 1.18-1.37 (m, 60H) 0.82-0.93 (m, 6H).

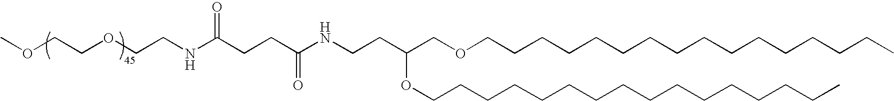

### Example 59

### N-[3,4-bis(hexadecyloxy)butyl]-N′-3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylsuccinamide

Example 59 was prepared using the same procedure described for Example 52F, substituting RAPP 12 2000-35 (Rapp Polymere) for mPEG2000-SCM. MS (MALDI) m/z 2584.3; ¹H NMR (300 MHz, CDCl₃) δ ppm 6.43-6.61 (m, 2H) 3.60-3.68 (m, 200H) (m, 16H) 2.42-2.57 (m, 4H) 1.49-1.85 (m, 6H) 1.19-1.35 (m, 52H) 0.82-0.92 (m, 6H).

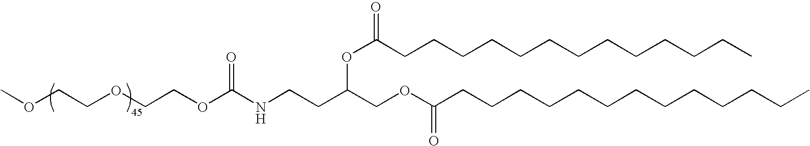

### Example 60

### 6-oxo-2-(tetradecanoyloxy)-7,10,13,16,19,22,25,28,31,34,37,40,43,46,49,52,55,58,61,64,67,70,73,76,79,82,85,88,91,94,9 7,100,103,106,109,112,115,118,121,124,127,130,133,136,139,142,145-heptatetracontaoxa-5-azahexatetracontahect-1-yl myristate

Example 60 was prepared using the same procedure described for Example 52F, substituting mPEG-NPC (Creative PEGWorks) for mPEG2000-SCM (Laysan Bio, Inc.). MS (MALDI) m/z 2588.5; ¹H NMR (300 MHz, CDCl₃) δ ppm 5.14 (m, 1H) 4.17-4.26 (m, 3H) 4.01-4.11 (m, 1H) 3.83-3.91 (m, 1H) 3.60-3.71 (m, 180H) 3.48-3.60 (m, 4H) 3.35-3.44 (m, 5H) 2.23-2.37 (m, 4H) 1.62-1.86 (m, 6H) 1.21-1.37 (m, 40H) 0.83-0.93 (m, 6H).

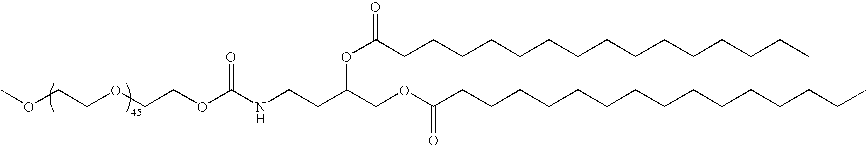

### Example 61

### 6-oxo-2-(palmitoyloxy)-7,10,13,16,19,22,25,28,31,34,37,40,43,46,49,52,55,58,61,64,67,70,73,76,79,82,85,88,91,94,9 7,100,103,106,109,112,115,118,121,124,127,130,133,136,139,142,145-heptatetracontaoxa-5-azahexatetracontahect-1-yl palmitate

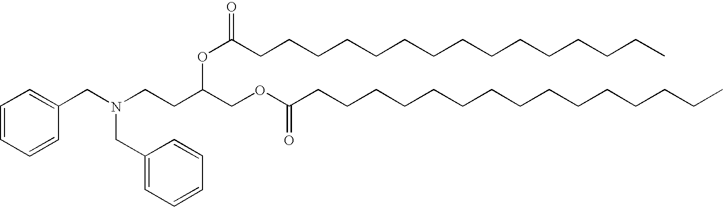

### Example 61A

### 4-(dibenzylamino)butane-1,2-diyl dipalmitate

Example 61A was prepared using the same procedure described for Example 52D, substituting hexadecanoic acid for tetradecanoic acid. MS (ESI) m/z 762.4 (M+H)⁺; ¹H NMR (300 MHz, CDCl₃) δ ppm 7.15-7.42 (m, 10H) 5.06-5.21 (m, 1H) 4.12 (dd, J=11.90, 3.57 Hz, 1H) 3.91 (dd, J=11.90, 5.95 Hz, 1H) 3.43-3.62 (m, 4H) 2.34-2.58 (m, 2H) 2.25 (t, J=7.34 Hz, 2H) 2.01-2.16 (m, 2H) 1.77 (q, J=7.14 Hz, 2H) 1.40-1.64 (m, 4H) 1.14-1.37 (m, 48H) 0.82-0.95 (m, 6H).

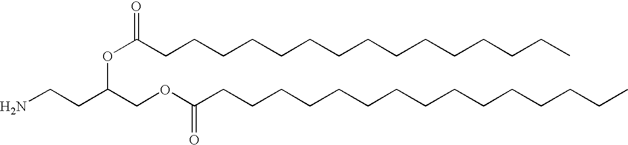

### Example 61B

### 4-aminobutane-1,2-diyl dipalmitate

Example 61B was prepared using the same procedure described for Example 52E, substituting Example 61A for 52D. MS (ESI) m/z 482.6 (M+H)⁺.

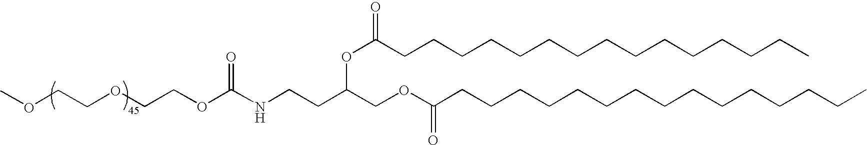

### Example 61C

### 6-oxo-2-(palmitoyloxy)-7,10,13,16,19,22,25,28,31,34,37,40,43,46,49,52,55,58,61,64,67,70,73,76,79,82,85,88,91,94,9 7,100,103,106,109,112,115,118,121,124,127,130,133,136,139,142,145-heptatetracontaoxa-5-azahexatetracontahect-1-yl palmitate

Example 61C was prepared using the same procedure described for Example 52F, substituting Example 61B for Example 52E and substituting mPEG-NPC (Creative PEGWorks) for mPEG2000-SCM (Laysan Bio, Inc.). MS (MALDI) m/z 2689.0; ¹H NMR (300 MHz, CDCl₃) δ ppm 5.09-5.19 (m, 1H) 4.17-4.26 (m, 3H) 4.01-4.11 (m, 1H) 3.73-3.91 (m, 1H) 3.61-3.70 (m, 180H) 3.48-3.60 (m, 4H) 3.35-3.44 (m, 5H) 2.23-2.36 (m, 4H) 1.54-1.84 (m, 6H) 1.21-1.36 (m, 48H) 0.82-0.93 (m, 6H).

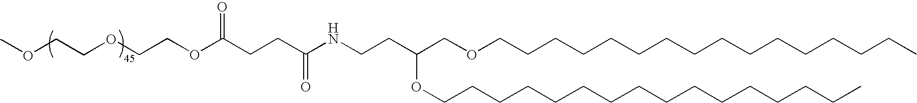

### Example 62

### 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate

Example 54B (100 mg) and mPEG-COOH (278 mg, PSA-288, Creative PEGWorks) were combined in dichloromethane (2 mL). N¹-((ethylimino)methylene)-N³,N³-dimethylpropane-1,3-diamine hydrochloride (346 mg) was added followed by 4-(dimethylamino)pyridine (2 mg). The mixture was stirred overnight at room temperature then loaded directly onto a 4 g silica gel column (Analogix) and purified (Analogix 280, dichloromethane:methanol 0-20%). (MALDI) m/z 2628.4; ¹H NMR (300 MHz, CDCl₃) δ ppm 4.19-4.28 (m, 2H) 3.83-3.92 (m, 1H) 3.65 (none, 180H) 3.36-3.59 (m, 16H) 2.69 (t, J=6.78 Hz, 2H) 2.43 (t, J=6.95 Hz, 2H) 1.47-1.71 (m, 6H) 1.22-1.32 (m, 52H) 0.84-0.92 (m, 6H).

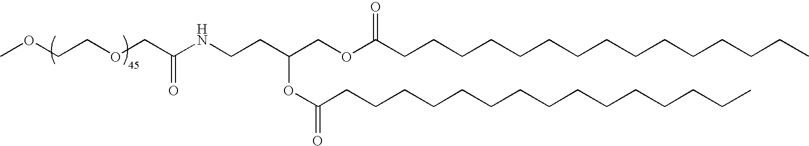

### Example 63

### 6-oxo-2-(palmitoyloxy)-8,11,14,17,20,23,26,29,32,35,38,41,44,47,50,53,56,59,62,65,68,71,74,77,80,83,86,89,92,95,9 8,101,104,107,110,113,116,119,122,125,128,131,134,137,140,143-hexatetracontaoxa-5-azatetratetracontahect-1-yl palmitate

This example was prepared using the same procedure described for Example 52F, substituting Example 61B for Example 52E. MS (MALDI) m/z 2835.3; ¹H NMR (300 MHz, CDCl₃) δ ppm 5.07-5.20 (m, 1H) 4.24 (dd, J=11.90, 3.57 Hz, 1H) 4.06 (dd, J=11.90, 6.35 Hz, 1H) 3.98 (s, 2H) 3.61-3.68 (m, 180H) 3.49-3.60 (m, 4H) 3.36-3.48 (m, 5H) 2.25-2.36 (m, 4H) 1.77-1.87 (m, 2H) 1.26 (m, 48H) 0.83-0.93 (m, 6H).

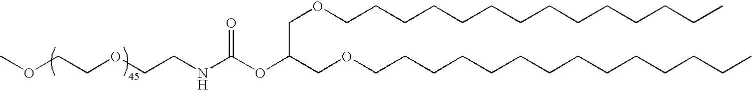

### Example 64

### 2-(tetradecyloxy)-1-((tetradecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate

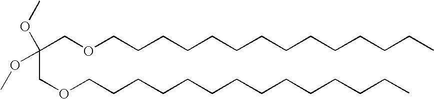

### Example 64A

### 1-(2,2-dimethoxy-3-(tetradecyloxy)propoxy)tetradecane

To a solution of 2,2-dimethoxypropane-1,3-diol (1 g) in toluene (30 mL) at 0° C. was added NaH (1.484 g). The mixture was stirred at room temperature for 1 hour. The mixture was cooled to 0° C., and 1-bromotetradecane (4.99 mL) was added. The mixture was heated at reflux for 2 hours. The mixture was cooled to 0° C., and ethanol was added until it became clear. The mixture was concentrated. The concentrate was taken up in dichloromethane and dried onto silica gel. The silica was loaded into an Analogix DASI module, and the product was isolated by flash chromatography (Analogix, SF65×200 g, 2% ethyl acetate/hexanes for six column volumes, then 4% ethyl acetate/hexanes until major product eluted). MS (ESI) m/z 512 (M-CH₃+1).

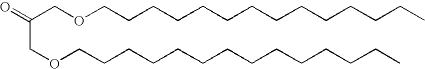

### Example 64B

### 1,3-bis(tetradecyloxy)propan-2-one

To a solution of 1-(2,2-dimethoxy-3-(tetradecyloxy)propoxy)tetradecane (2.2 g) in tetrahydrofuran (60 mL) was added 6N hydrogen chloride (5.55 mL). The mixture was stirred at room temperature overnight then concentrated. The concentrate was taken up in ethyl acetate, washed with saturated NaHCO₃, dried over Na₂SO₄, filtered, and concentrated. The concentrate was dissolved in dichloromethane and concentrated onto silica gel. The silica gel was loaded into an Analogix DASI module, and the product was isolated by flash chromatography (Analogix, SF65×200 g, 2% ethyl acetate/hexanes for six column volumes, then 4% ethyl acetate/hexanes until the product eluted. MS (ESI) m/z 500.4 (M+18)⁺.

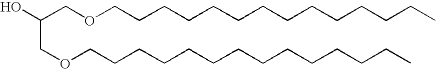

### Example 64C

### 1,3-bis(tetradecyloxy)propan-2-ol

To a solution of 1,3-bis(tetradecyloxy)propan-2-one (0.68 g) in tetrahydrofuran (13 mL) at 0° C. was added sodium borohydride (0.085 g) and water (0.867 mL). The mixture was stirred at room temperature for 1 hour, cooled to 0° C., and quenched with 1N HCl. The mixture was extracted with ethyl acetate. The extract was dried over Na₂SO₄, filtered and concentrated. The concentrate was purified by flash chromatography (1:5 ethyl acetate/hexanes). MS (ESI) m/z 484 (M+1)⁺, 502 (M+18)⁺.

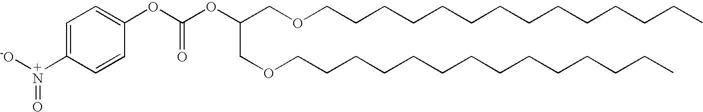

### Example 64D

### 1,3-bis(tetradecyloxy)propan-2-yl 4-nitrophenyl carbonate

To a solution of 1,3-bis(tetradecyloxy)propan-2-ol (0.3 g) in dichloromethane (3 mL) at 0° C. were added triethlyamine (0.129 mL) and 4-nitrophenyl carbonochloridate (0.137 g). The mixture was stirred at room temperature overnight and concentrated. The concentrate was purified by flash chromatography (1:10 ethyl acetate/hexanes). ¹H NMR (300 MHz, CDCl₃) δ ppm 8.24-8.30 (m, 2H), 7.37-7.42 (m, 2H), 5.06-5.13 (m, 1H), 3.67 (d, J=15.16 Hz, 4H), 3.41-3.55 (m, 4H), 1.55-1.60 (m, 4H), 1.19-1.38 (m, 44H), 0.85-0.90 (m, 6H).

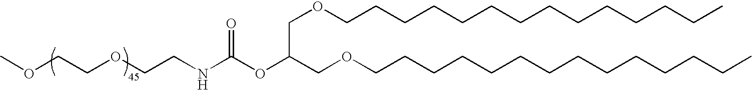

### Example 64E

### 2-(tetradecyloxy)-1-((tetradecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate

To a solution of CH₃O-PEG2000-NH₂ (12 2000-2 Rapp Polymere, 0.2 g) in dichloromethane (1 mL) were added 1,3-bis(tetradecyloxy)propan-2-yl 4-nitrophenyl carbonate (0.195 g) and triethylamine (0.015 g). The mixture was stirred at room temperature overnight. The mixture was directly purified by flash chromatography (5-20% methanol/dichloromethane). ¹H NMR (300 MHz, CDCl₃) δ ppm 3.53-3.66 (m, 180H), 3.32-3.49 (m, 9H), 3.38 (s, 3H), 1.51-1.59 (m, 4H), 1.21-1.35 (m, 44H), 0.86-0.90 (m, 6H); MS (MALDI) m/z 2549.

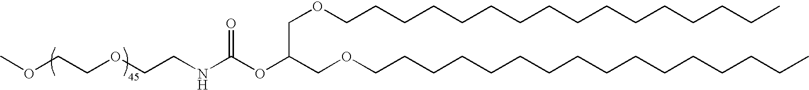

### Example 65

### 2-(hexadecyloxy)-1-((hexadecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,1 17,120,123,126,129,132,135,138-hexatetracontaoxanonatriacontahect-1-ylcarbamate

This Example was prepared as described in Example 64, substituting hexadecyl methanesulfonate for 1-bromotetradecane in Example 64A. ¹H NMR (300 MHz, CDCl₃) δ ppm 3.54-3.66 (m, 180H), 3.32-3.49 (m, 9H), 3.38 (s, 3H), 1.51-1.59 (m, 4H), 1.21-1.36 (m, 48H), 0.86-0.90 (m, 6H); MS (MALDI) m/z 2614.

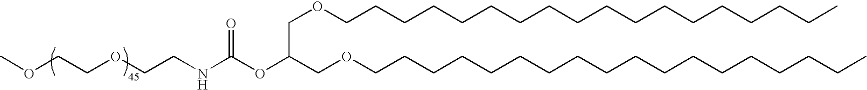

### Example 66

### 2-(octadecyloxy)-1-((octadecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,1 17,120,123,126,129,132,135,138-hexatetracontaoxanonatriacontahect-1-ylcarbamate

This Example was prepared as described in Example 64 substituting octadecyl methanesulfonate for 1-bromotetradecane in Example 64A. ¹H NMR (300 MHz, CDCl₃) δ ppm 3.52-3.66 (m, 180H), 3.32-3.49 (m, 9H), 3.38 (s, 3H), 1.51-1.59 (m, 4H), 1.21-1.36 (m, 52H), 0.86-0.90 (m, 6H); MS (MALDI) m/z 2557.

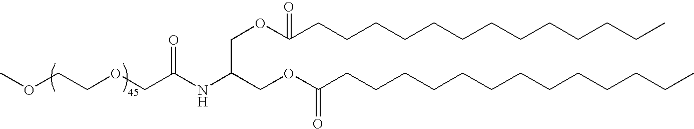

### Example 67

### 2-2,5,8,11,14,17,20,23,26,29,32,35,38,41,44,47,50,53,56,59,62,65,68,71,74,77,80,83,86,89,92, 94,97,100,103,106,109,112,115,118,121,124,127,130,133,136-hexatetracontaoxaoctatriacontahectanamidopropane-1,3-diyl ditetradecanoate

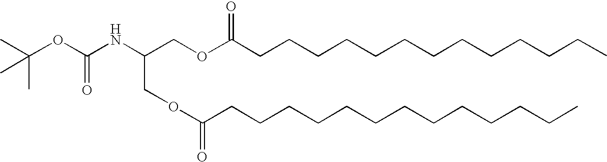

### Example 67A

### 2-(tert-butoxycarbonylamino)propane-1,3-diyl ditetradecanoate

To a solution of tetradecanoic acid (1.051 g) in dichloromethane (10 mL) at 0° C. were added tert-butyl 1,3-dihydroxypropan-2-ylcarbamate (0.40 g), 4-(dimethylamino)pyridine (0.562 g), N-methylmorpholine (1.150 mL), and 1-ethyl-3-(3-(dimethylamino)propyl)carbodiimide hydrochloride (0.882 g). The mixture was stirred at room temperature overnight. The mixture was partitioned between water and dichloromethane. The aqueous layer was extracted with dichloromethane. The extract were dried over Na₂SO₄, filtered, and concentrated. The concentrate was purified by flash chromatography (1:10 ethyl acetate/hexanes). MS (ESI) m/z 512.4 (M-CO₂-tert-butyl+1)⁺.

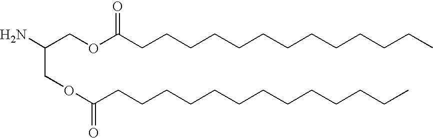

### Example 67B

### 2-aminopropane-1,3-diyl ditetradecanoate

To a solution of 2-(tert-butoxycarbonylamino)propane-1,3-diyl ditetradecanoate in dichloromethane (10 mL) was added trifluoroacetic acid. The mixture was stirred at room temperature for 2 hours then concentrated. The concentrate was purified by flash chromatography. MS (ESI) m/z 512.4 (M+1)⁺.

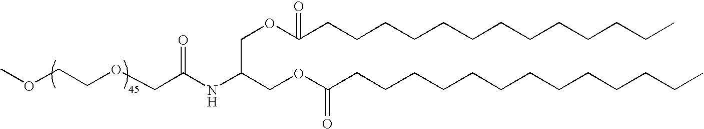

### Example 67C

### 2-2,5,8,11,14,17,20,23,26,29,32,35,38,41,44,47,50,53,56,59,62,65,68,71,74,77,80,83,86,89,92, 94,97,100,103,106,109,112,115,118,121,124,127,130,133,136-hexatetracontaoxaoctatriacontahectanamidopropane-1,3-diyl ditetradecanoate

To a flask was charged with mPEG2000-SCM (Laysan, 0.2 g) and 2-aminopropane-1,3-diyl ditetradecanoate (0.077 g) was added dichloromethane (2 mL). The mixture was stirred at room temperature overnight and concentrated. The concentrate was purified by flash chromatography (5-20% methanol/dichloromethane). ¹H NMR (300 MHz, CDCl₃) δ ppm 4.11-4.21 (m, 4H), 4.01 (s, 2H), 3.53-3.68 (m, 180H), 3.39-3.42 (m, 1H), 3.38 (s, 3H), 2.31 (t, J=7.46 Hz, 4H), 1.57-1.64 (m, 4H), 1.20-1.37 (m, 40H), 0.85-0.90 (m, 6H); MS (MALDI) m/z 2632.

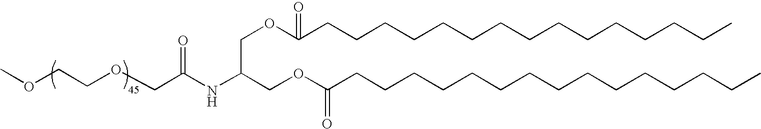

### Example 68

### 2-2,5,8,11,14,17,20,23,26,29,32,35,38,41,44,47,50,53,56,59,62,65,68,71,74,77,80,83,86,89,92, 94,97,100,103,106,109,112,115,118,121,124,127,130,133,136-hexatetracontaoxaoctatriacontahectanamidopropane-1,3-diyl dipalmitate

This Example was prepared as described in Example 67, substituting hexadecanoic acid for tetradecanoic acid in Example 67A. ¹H NMR (300 MHz, CDCl₃) δ ppm 4.10-4.21 (m, 4H), 4.01 (s, 2H), 3.53-3.69 (m, 180H), 3.39-3.42 (m, 1H), 3.38 (s, 3H), 2.31 (t, J=7.63 Hz, 4H), 1.56-1.63 (m, 4H), 1.20-1.33 (m, 44H), 0.85-0.91 (m, 6H); MS (MALDI) m/z 2732.

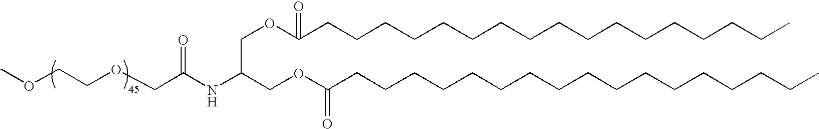

### Example 69

### 2-2,5,8,11,14,17,20,23,26,29,32,35,38,41,44,47,50,53,56,59,62,65,68,71,74,77,80,83,86,89,92, 94,97,100,103,106,109,112,115,118,121,124,127,130,133,136-hexatetracontaoxaoctatriacontahectanamidopropane-1,3-diyl distearate

This Example was prepared as in Example 67, substituting octadecanoic acid for tetradecanoic acid in Example 67A. ¹H NMR (300 MHz, CDCl₃) δ ppm 4.10-4.21 (m, 4H), 4.01 (s, 2H), 3.53-3.69 (m, 180H), 3.39-3.42 (m, 1H), 3.38 (s, 3H), 2.31 (t, J=7.63 Hz, 4H), 1.57-1.63 (m, 4H), 1.21-1.33 (m, 48H), 0.85-0.90 (m, 6H); MS (MALDI) m/z 2832.

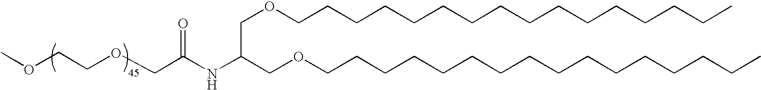

### Example 70

### N-(2-(hexadecyloxy)-1-((hexadecyloxy)methyl)ethyl)-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

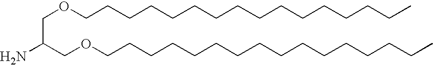

### Example 70A

In a 100 mL round-bottomed flask was added N-Boc-serinol (1,1-dimethylethyl (2-hydroxy-1-(hydroxymethyl)ethyl)carbamate) (2.0 g) and sodium hydride (1.255 g) in N,N-dimethylformamide (50 mL). The mixture was cooled using an ice/water bath, and 1-bromohexadecane (7.98 g) was added to it. The mixture was heated at 70° C. overnight, then cooled to room temperature. The mixture was cooled to 0° C. and quenched with a few drops of cold water. The mixture was diluted with saturated ammonium chloride (50 mL). The aqueous layer was extracted with ethyl acetate, and the extract was washed with brine, dried over Na₂SO₄, and concentrated. The concentrate was added to a silica gel column and was eluted with ethyl acetate/hexane (1:9). The product, tert-butyl 1,3-bis(hexadecyloxy)propan-2-ylcarbamate, was directly used for the next step.

In a 100 mL round-bottomed flask was added tert-butyl 1,3-bis(hexadecyloxy)propan-2-ylcarbamate (5.0 g) and CH₂Cl₂ (40 mL). Trifluoroacetic acid (20 mL) was then added dropwise. The mixture was stirred under nitrogen for 3 hours and concentrated. The concentrate was added to a silica gel column and eluted with CH₂Cl₂/methanol (9:1). The product was dried under vacuum. ¹H NMR (300 MHz, CDCl₃) δ ppm 3.53-3.63 (m, 4H), 3.42-3.46 (t, 4H), 3.23 (m, 1H), 2.92-2.97 (m, 2H), 1.53-1.64 (m, 4H), 1.18-1.40 (m, 52H), 0.86-0.90 (t, 6H). MS (ESI) m/z 540.6 (M+1)⁺.

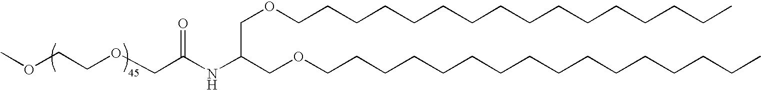

### Example 70B

Into a 40 mL glass vial was added 1,3-bis(hexadecyloxy)propan-2-amine (1.75 g) and mPEG2000-SCM (Laysan, 0.25 g, 1.081 mmol) in CH₂Cl₂ (10 mL). Triethylamine (0.50 mL) was added dropwise. The reaction solution was stirred under nitrogen for one day. The crude product was added to a silica gel column and was eluted with CH₂Cl₂/methanol (9:1). The product was dried under vacuum. ¹H NMR (300 MHz, CDCl₃) δ ppm 4.17-4.18 (m, 1H), 4.14 (s, 2H), 3.86-3.88 (m, 4H), 3.74-3.76 (t, 4H), 3.61-3.71 (m, 180H), 3.38 (s, 3H), 1.51-1.59 (m, 4H), 1.23-1.32 (m, 56H), 0.86-0.90 (m, 6H); MS (MALDI) m/z 2700.

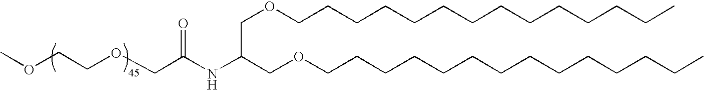

### Example 71

### N-(2-(tetradecyloxy)-1-((tetradecyloxy)methyl)ethyl)-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

This Example was prepared as described in Example 70, substituting 1-bromotetradecane for 1-bromohexadecane in Example 70A. ¹H NMR (300 MHz, CDCl₃) δ ppm 4.18 (m, 1H), 4.10 (s, 2H), 3.86-3.89 (m, 4H), 3.72-3.75 (t, 4H), 3.61-3.71 (m, 180H), 3.38 (s, 3H), 1.50-1.60 (m, 4H), 1.24-1.30 (m, 48H), 0.86-0.90 (m, 6H); MS (MALDI) m/z 2400.

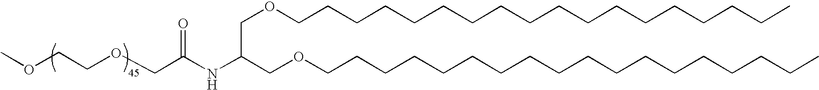

### Example 72

### N-(2-(octadecyloxy)-1-((octadecyloxy)methyl)ethyl)-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

This Example was prepared as described in Example 70, substituting 1-bromooctadecane hexadecane for 1-bromotetradecane in Example 70A. ¹H NMR (300 MHz CDCl₃) δ ppm 4.14-4.20 (m, 1H), 4.08 (s, 2H), 3.86-3.89 (t, 4H), 3.71-3.75 (m, 4H), 3.61-3.70 (m, 180H), 3.38 (s, 3H), 1.50-1.56 (m, 4H), 1.20-1.30 (m, 64H), 0.86-0.90 (m, 6H); MS (MALDI) m/z 2900.

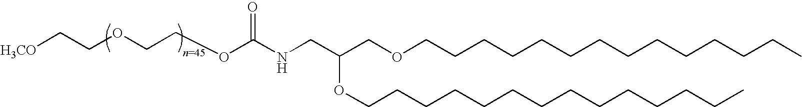

### Example 73

### N-(2,3-dimyristyloxypropyl)carbamate polyethyleneglycol-2000 methyl ether

Example 58 was prepared using the known synthetic route; see: Heyes, J.; Hall, K.; Tailor, V.; Lenz, R.; MacLachlan, I. J. Controlled Release 2006, 112, 280-290.

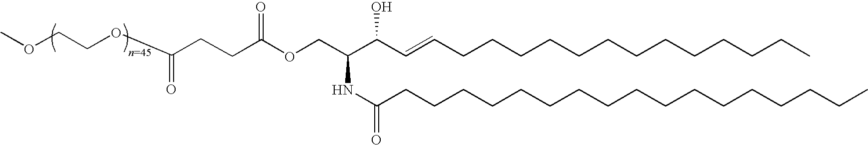

### Example 74

### (2S,3R,E)-3-hydroxy-2-stearamidooctadec-4-enyl polyethyleneglycol-2000 methyl ether succinate

Example 74 was prepared using a known synthetic route; see: U.S. Pat. No. 5,820,873.

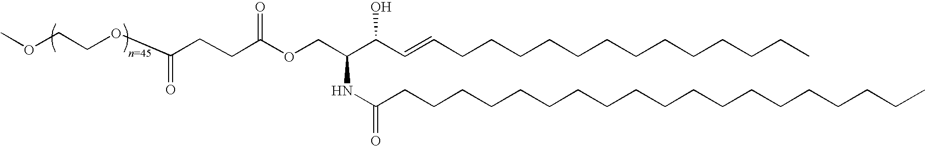

### Example 75

### (2S,3R,E)-3-hydroxy-2-icosanamidooctadec-4-enyl polyethyleneglycol-2000 methyl ether succinate

Example 75 was prepared using a known synthetic route; see: U.S. Pat. No. 5,820,873.

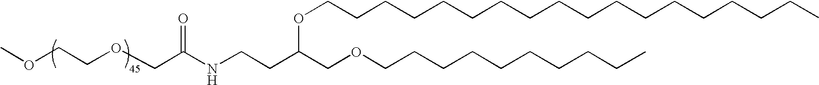

### Example 77

### N-[4-(decyloxy)-3-(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

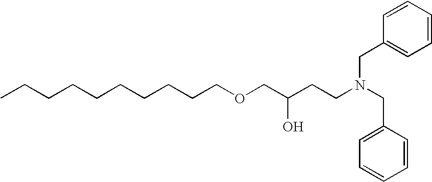

### 1-(decyloxy)-4-(dibenzylamino)butan-2-ol

### Example 77A

Into a 100 mL round-bottomed flask was added EXAMPLE 52C (1 g, 3.50 mmol) and the mixture was dissolved in tetrahydrofuran (11.68 ml), followed by NaH (0.252 g, 10.51 mmol) to give a suspension. The solution was stirred at room temperature for 30 minutes. 1-Bromodecane (1.598 ml, 7.71 mmol) was added at room temperature, then the mixture was warmed to 60° C. for 12 hours. The reaction was diluted with N,N-dimethylformamide and heated to 90° C. overnight. The reaction was cooled to room temperature, and quenched with water. The reaction was poured into ethyl acetate, and the resulting layers were separated. The organics were collected, dried over MgSO₄, filtered, and reduced in vacuo. The residue was purified via an Analogix flash chromatography system (hexanes:ethyl acetate) to afford the title compound. LC/MS m/z 426 (M+H)⁺.

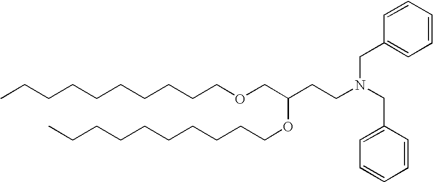

### N,N-dibenzyl-3,4-bis(decyloxy)butan-1-amine

### Example 77B

Into a 100 mL round-bottomed flask was added EXAMPLE 1C (1 g, 3.50 mmol) and the mixture was dissolved in tetrahydrofuran (11.68 ml), followed by NaH (0.252 g, 10.51 mmol) to give a suspension. The solution was stirred at room temperature for 30 minutes. 1-Bromodecane (1.598 ml, 7.71 mmol) was added at room temperature, then the mixture was warmed to 60° C. for 12 hours. The reaction was diluted with N,N-dimethylformamide and heated to 90° C. overnight. The reaction was cooled to room temperature, and quenched with water. The reaction was poured into ethyl acetate, and the resulting layers were separated. The organics were collected, dried over MgSO₄, filtered, and reduced in vacuo. The residue was purified via an Analogix flash chromatography system (hexanes:ethyl acetate) to afford the title compound. LC/MS m/z 566 (M+H)⁺.

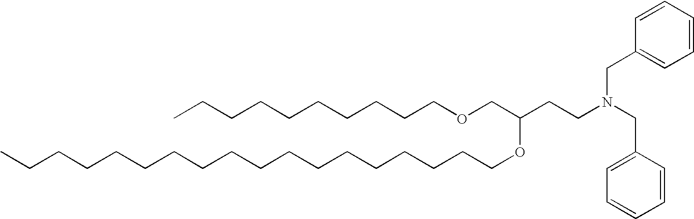

### N,N-dibenzyl-4-(decyloxy)-3-(octadecyloxy)butan-1-amine

### Example 77C

Into a 15 mL vial was added EXAMPLE 77A (0.52 g, 1.222 mmol) and NaH (0.088 g, 3.67 mmol) in N,N-dimethylformamide (6.11 ml) to give a suspension, and the reaction stirred for 15 minutes at room temperature. Octadecyl methanesulfonate (0.468 g, 1.344 mmol) was added and the reaction was heated to 90° C. overnight. The reaction was cooled to room temperature, quenched with water, and diluted with diethyl ether. The organics were separated, and the aqueous layer was extracted with diethyl ether. The organic layers were combined, dried over MgSO₄, filtered and reduced in vacuo. The residue was purified via Analogix using a gradient elution (100% to 90% Hexane/ethyl acetate) to afford the title compound. MS (ESI) m/z 678.8 (M+H)⁺.

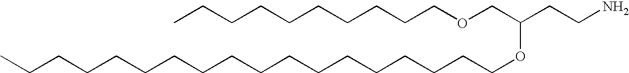

### 4-(decyloxy)-3-(octadecyloxy)butan-1-amine

### Example 77D

Into a 50 mL round-bottomed flask was added EXAMPLE 77C (0.3 g, 0.442 mmol) and Pd/C (0.047 g, 0.044 mmol) in CH₂Cl₂ (2.212 ml)/methanol (2.212 ml) to give a black suspension, the system was purged via vacuum, then 1 atm H₂. The process was repeated 3 times. The reaction was stirred at room temperature under 1 atm of H₂ for 18 hrs. The reaction was treated with Celite, filtered over Celite. The Celite pad was washed with CH₂Cl₂/MeOH. The organics were reduced in vacuo to afford a solid. The residue was purified via Analogix using a gradient elution (100% to 80% CH₂Cl₂/MeOH) to afford EXAMPLE 15D. MS (ESI) m/z 498.7 (M+H)⁺.

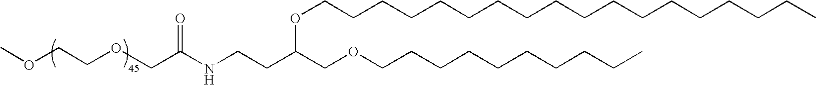

### Example 77E

### N-[4-(decyloxy)-3-(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

EXAMPLE 77D (75 mg, 0.151 mmol) and Hunig's base (30.1 uL) were combined in dichloromethane (2mL) at room temperature. mPEG-SCM (MW 2000, Laysan Bio, 172 mg, 0.086 mmol) was added to the solution and the mixture was stirred overnight at room temperature. The reaction mixture was loaded directly onto silica gel and purified by flash column chromatography (Analogix) (100% ethyl acetate, followed by 0-15% methanol in dichloromethane) to afford the title compound. MS (MALDI) m/z 2750.8; ¹H NMR (300 MHz, CHLOROFORM-D) δ ppm 3.98 (s, 2H) 3.85-3.90 (m, 1H) 3.61-3.72 (m, 180H) 3.36-3.60 (m, 11H) 1.25 (s, 44H) 0.83-0.93 (m, 6H).

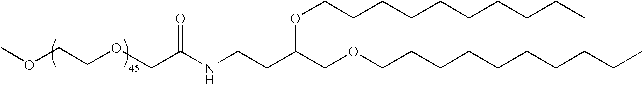

### Example 78

### N-[3,4-bis(decyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

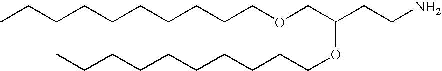

### 3,4-bis(decyloxy)butan-1-amine

### Example 78A

Into a 50 mL round-bottomed flask was added EXAMPLE 77B (0.636 g, 1.124 mmol) and Pd/C (0.239 g, 0.225 mmol) in methanol (1.873 ml)/CH₂Cl₂ (1.873 ml) to give a suspension. The reaction mixture was purged with H₂, and evacuated in vacuo. This cycle was repeated 3 times, and the mixture was allowed to stir under 1 atm of H₂ at room temperature overnight. The mixture was treated with diatomaceous earth, and filtered over diatomaceous earth. The diatomaceous earth was washed with CH₂Cl₂ and methanol. The organics were reduced in vacuo. The residue was purified via Analogix using a gradient elution (0 to 20% methanol in CH₂Cl₂) to afford the title compound. MS (ESI) m/z 386.3 (M+H)⁺.

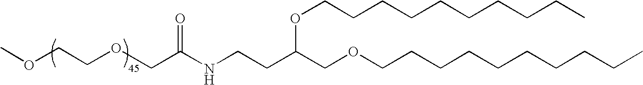

### Example 78B

### N-[3,4-bis(decyloxy)butyl]-2,5,8,11,14,17,20,23,26,29,32,35,38,41,44,47,50,53,56,59,62,65,68,71,74,77,80,83,86,89,92, 95,98,101,104,107,1 10,113,116,1 19,122,125,128,131,134,137-hexatetracontaoxanonatriacontahectan-139-amide

Example 78B was prepared using the same procedure described for Example 52F, substituting Example 78A for Example 52E. MS (MALDI) m/z 2726.3; ¹H NMR (300 MHz, CHLOROFORM-D) δ ppm 3.98 (s, 2H) 3.87 (dd, J=5.76, 4.07 Hz, 1H) 3.61-3.68 (m, 180H) 3.36-3.59 (m, 11H) 1.50-1.61 (m, 6H) 1.26 (s, 28H) 0.83-0.93 (m, 6H).

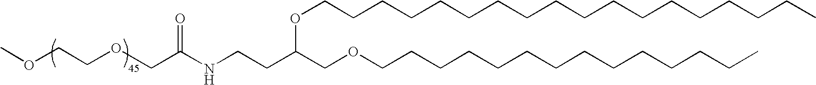

### Example 79

### N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89,92, 95,98,101,104,107,1 10,113,116,1 19,122,125,128,131,134,137-hexatetracontaoxanonatriacontahectan-139-amide

Example 79 was prepared using the same procedure described for Example 77, substituting 1-bromotetradecane for 1-bromodecane in Example 77A. MS (MALDI) m/z 2895.9; 1H NMR (300 MHz, CHLOROFORM-D) δ ppm 3.98 (s, 2H) 3.84-3.92 (m, 1H) 3.62-3.68 (m, 180H) 3.35-3.60 (m, 11H) 1.46-1.57 (m, 6H) 1.25 (s, 52H) 0.83-0.92 (m,6H).

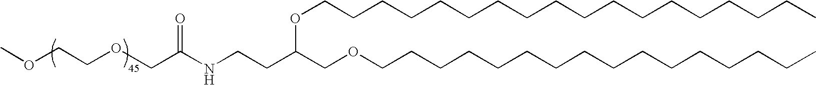

### Example 80

### N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

Example 80 was prepared using the same procedure described for Example 77, substituting 1-bromohexadecane for 1-bromodecane in Example 77A. MS (MALDI) m/z 2878.5; ¹H NMR (300 MHz, CHLOROFORM-D) δ ppm 3.98 (s, 2H) 3.84-3.91 (m, 1H) 3.62-3.67 (m, 180H) 3.34-3.60 (m, 11H) 1.54 (d, J=7.46 Hz, 6H) 1.21-1.35 (m, 56H) 0.84-0.91 (m,6H).

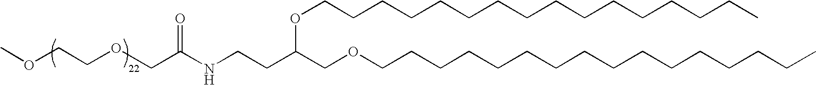

### Example 81

### N-[3,4-bis(hexadecyloxy)butyl]-2,5,8,11,14,17,20,23,26,29,32,35,38,41,44,47,50,53,56,59,62,65,68-tricosaoxaheptacontan-70-amide

Example 81 was prepared using the same procedure described for Example 52F, substituting mPEG1000-SCM (Laysan Bio, Inc.) for mPEG2000-SCM (Laysan Bio, Inc.) and Example 54B for Example 52E. MS (MALDI) m/z 1794.3; ¹H NMR (300 MHz, CHLOROFORM-D) δ ppm 3.98 (s, 2H) 3.82-3.91 (m, 1H) 3.62-3.68 (m, 88H) 3.35-3.61 (m, 11H) 1.48-1.60 (m, 6H) 1.20-1.36 (m, 52H) 0.83-0.93 (m, 6H).

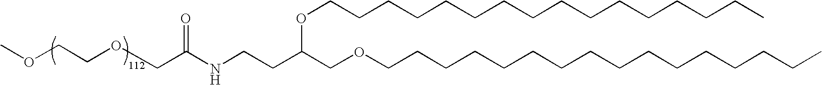

### Example 82

### N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143,146,149,152,155,158, 161,164,167,170,173,176,179,182,185,188,191,194,197,200,203,206,209,212,215,218,221,2 24,227,230,233,236,239,242,245,248,251,254,257,260,263,266,269,272,275,278,281,284,28 7,290,293,296,299,302,305,308,311,314,317,320,323,326,329,332,335,338-113oxa340n-340-amide

Example 82 was prepared using the same procedure described for Example 52F, substituting mPEG5000-SCM (Laysan Bio, Inc.) for mPEG2000-SCM (Laysan Bio, Inc.) and Example 54B for Example 52E. MS (MALDI) m/z 5978.3; ¹H NMR (300 MHz, CHLOROFORM-D) δ ppm 3.98 (s, 2H) 3.87 (dd, J=5.93, 4.24 Hz, 1H) 3.61-3.68 (m, 448H) 3.35-3.60 (m, 11H) 1.46-1.62 (m, 6H) 1.25 (s, 52H) 0.82-0.92 (m, 6H).

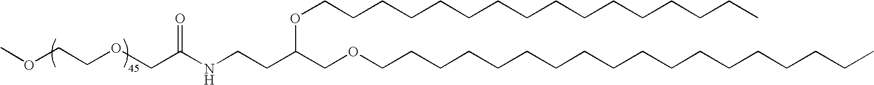

### Example 83

### N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide

Example 83 was prepared using the same procedure described for Example 77, substituting 1-bromooctadecane for 1-bromodecane in Example 77A and hexadecyl methanesulfonate for octadecyl methanesulfonate in Example 77B. MS (MALDI) m/z 2746.3; ¹H NMR (300 MHz, CHLOROFORM-D) δ ppm 3.98 (s, 2H) 3.87 (dd, J=5.76, 4.07 Hz, 1H) 3.60-3.69 (m, 180H) 3.36-3.61 (m, 11H) 1.55 (s, 6H) 1.25 (s, 56H) 0.83-0.93 (m, 6H).

We claim:

1. A cationic lipid having Formula (I) 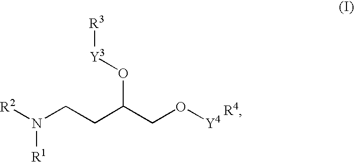
Formula (II) 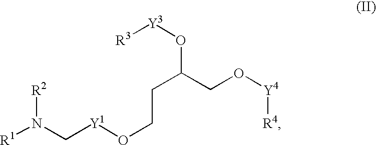
Formula (III) 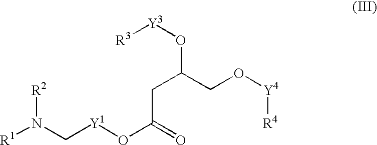
or
Formula (IV) 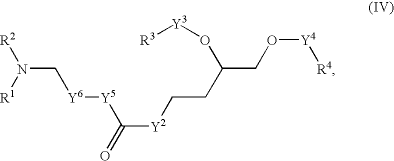
wherein Y¹ is C₁-C₆ alkylene;
Y² is CH₂, NH or O;
Y³ is a bond or C(O);
Y⁴ is a bond or C(O);
Y⁵ is CH₂, NH or O;
Y⁶ is a bond or C₁-C₆ alkylene;
R¹ and R² are independently H, cycloalkyl, cycloalkenyl or R⁵; or
R¹ and R², together with the nitrogen to which they are attached, are heterocycloalkyl or heteroaryl;
one of R³ and R⁴ is H, and the other is C₁₄-C₂₀-alkenyl, or C₁₄-C₂₀-alkyl; or R³ and R⁴ are independently selected C₁₄-C₂₀-alkenyl, or C₁₄-C₂₀-alkyl; or
R³ and R⁴ together are CR²⁰R²¹, wherein R²⁰ is H and R²¹ is C₁₄-C₂₀-alkenyl, C₁₄-C₂₀-alkyl, or CH₂O—C₁₄-C₂₀-alkenyl; or R²⁰ and R²¹ are independently selected C₁₄-C₂₀-alkenyl, C₁₄-C₂₀-alkyl, or CH₂O—C₁₄-C₂₀-alkenyl;
R⁵ is alkyl, which is unsubstituted or substituted with one or more R⁶, OR⁶, SR⁶, S(O)R⁶, SO₂R⁶, C(O)R⁶, CO(O)R⁶, OC(O)R⁶, OC(O)OR⁶, NH₂, NHR⁶, N(R⁶)₂, NHC(O)R⁶, NR⁶C(O)R⁶, NHS(O)₂R⁶, NR⁶S(O)₂R⁶, NHC(O)OR⁶, NR⁶C(O)OR⁶, NHC(O)NH₂, NHC(O)NHR⁶, NHC(O)N(R⁶)₂, NR⁶C(O)NHR⁶, NR⁶C(O)N(R⁶)₂, C(O)NH₂, C(O)NHR⁶, C(O)N(R⁶)₂, C(O)NHOH, C(O)NHOR⁶, C(O)NHSO₂R⁶, C(O)NR⁶SO₂R⁶, SO₂NH₂, SO₂NHR⁶, SO₂N(R⁶)₂, C(O)H, C(O)OH, C(N)NH₂, C(N)NHR⁶, C(N)N(R⁶)₂, CNOH, CNOCH₃, OH, (O), CN, N₃, NO₂, CF₃, CF₂CF₃, OCF₃, OCF₂CF₃, F, Cl, Br or I;
R⁶ is R⁷, R⁸, R⁹, or R¹⁰;
R⁷ is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R⁸ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R⁹ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R¹⁰ is alkyl, alkenyl or alkynyl;
wherein each foregoing cyclic moiety is independently unsubstituted or substituted with one or more R¹¹, OR¹¹, SR¹¹, S(O)R¹¹, SO₂R¹¹, C(O)R¹¹, CO(O)R¹¹, OC(O)R¹¹, OC(O)OR¹¹, NH₂, NHR¹¹, N(R¹¹)₂, NHC(O)R¹¹, NR¹¹C(O)R¹¹, NHS(O)₂R¹¹, NR¹¹S(O)₂R¹¹, NHC(O)OR¹¹, NR¹¹C(O)OR¹¹, NHC(O)NH₂, NHC(O)NHR¹¹, NHC(O)N(R¹¹)₂, NR¹¹C(O)NHR¹¹, NR¹¹C(O)N(R¹¹)₂, C(O)NH, C(O)NHR¹¹, C(O)N(R¹¹)₂, C(O)NHOH, C(O)NHOR¹¹, C(O)NHSO₂R¹¹, C(O)NR¹¹SO₂R¹¹, SO₂NH₂, SO₂NHR¹¹, SO₂N(R¹¹)₂, C(O)H, C(O)OH, C(N)NH₂, C(N)NHR¹¹, C(N)N(R¹¹)₂, CNOH, CNOCH₃, OH, (O), CN, N₃, NO₂, CF₃, CF₂CF₃, OCF₃, OCF₂CF₃, F, Cl, Br or I;
R¹¹ is R¹², R¹³, R¹⁴, or R¹⁵;
R¹² is phenyl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R¹³ is heteroaryl which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R¹⁴ is cycloalkyl, cycloalkenyl, heterocycloalkyl or heterocycloalkenyl, each of which is unfused or fused with benzene, heteroarene, cycloalkane, cycloalkene, heterocycloalkane, or heterocycloalkene, each of which is unfused or fused with benzene, heteroarene, cycloalkane, cycloalkene, heterocycloalkane, or heterocycloalkene;
R¹⁵ is alkyl, alkenyl or alkynyl, each of which is unsubstituted or substituted with one or two of independently selected R¹⁶, OR^{16E}, SR¹⁶, S(O)₂R¹⁶, C(O)OH, NH₂, NHR¹⁶N(R¹⁶)₂, C(O)R¹⁶, C(O)NH₂, C(O)NHR¹⁶, C(O)N(R¹⁶)₂, NHC(O)R¹⁶, NR¹⁶C(O)R¹⁶, NHC(O)OR¹⁶, NR¹⁶C(O)OR¹⁶, OH, F, Cl, Br or I;
R¹⁶ is alkyl, alkenyl, alkynyl, or R¹⁷;
R¹⁷ is phenyl, heteroaryl, cycloalkyl, cycloalkenyl, heterocycloalkyl or heterocycloalkenyl;
wherein R¹², R¹³, R¹⁴, and R¹⁷ are independently unsubstituted or substituted with one or more R¹⁸, OR¹⁸, SR¹⁸, S(O)R¹⁸, SO₂R¹⁸, C(O)R8, CO(O)R8, OC(O)R¹⁸, OC(O)OR¹⁸, NH₂, NHR¹⁸, N(R¹⁸)₂, NHC(O)R¹⁸, NR¹⁸C(O)R¹⁸, NHS(O)₂R¹⁸, NR¹⁸S(O)₂R¹⁸, NHC(O)OR¹⁸, NR¹⁸C(O)OR¹⁸, NHC(O)NH₂, NHC(O)NHR¹⁸, NHC(O)N(R¹⁸)₂, NR¹⁸C(O)NHR¹⁸, NR¹⁸C(O)N(R¹⁸)₂, C(O)NH₂, C(O)NHR¹⁸, C(O)N(R¹⁸)₂, C(O)NHOH, C(O)NHOR , C(O)NHSO₂R¹⁸, C(O)NR¹⁸SO₂R¹⁸, SO₂NH₂, SO₂NHR¹⁸, SO₂N(R¹⁸)₂, C(O)H, C(O)OH, C(N)NH₂, C(N)NHR , C(N)N(R¹⁸)₂, CNOH, CNOCH₃, OH, (O), CN, N₃, NO₂, CF₃, CF₂CF₃, OCF₃, OCF₂CF₃, F, Cl, Br or I; and
R¹⁸ is alkyl, alkenyl, alkynyl, phenyl, heteroaryl, cycloalkyl, cycloalkenyl, heterocycloalkyl or heterocycloalkenyl.

2. A Cationic-Based Lipid Encapsulation System (CaBLES) comprising:
one or more (PEG)-lipid conjugates,
one or more non-cationic lipids, and
one or more cationic lipids of claim 1.

3. A Lipid-Based Particle, comprising:
one or more (PEG)-lipid conjugates,
one or more non-cationic lipids,
one or more cationic lipids of claim 1, and
one or more therapeutic agents.

4. The CaBLES of claim 2, or the Lipid-Based Particle of claim 3, wherein one or more cationic lipids are chosen from 1-{3,4-bis[(9Z,12Z)-octadeca-9,12-dienyloxy]butyl}piperidine, 4-{3,4-bis[(9Z,12Z)-octadeca-9,12-dienyloxy]butyl}morpholine, N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, 1-{3,4-bis[(9Z,12Z)-octadeca-9,12-dienyloxy]butyl}-4-phenylpiperazine, 1-{3,4-bis[(9Z,12Z)-octadeca-9,12-dienyloxy]butyl}-4-methylpiperazine, N-(2-methoxyethyl)-N-methyl-3,4-bis[(9Z,12Z)-octadeca-9,12-dienyloxy]butan-1-amine, 1-{3,4-bis[(9Z,12Z)-octadeca-9,12-dienyloxy]butyl}-4-(2-methoxyphenyl)piperazine, N-{3,4-bis[(9Z,12Z)-octadeca-9,12-dienyloxy]butyl}-N,N′,N′-trimethylethane-1,2-diamine, N-{3,4-bis[(9Z,12Z)-octadeca-9,12-dienyloxy]butyl}-N-methyl-N-(2-pyridin-2-ylethyl)amine, N-benzyl-N-{3,4-bis[(9Z,12Z)-octadeca-9,12-dienyloxy]butyl}-N-methylamine, N-{3,4-bis[(9Z,12Z)-octadeca-9,12-dienyloxy]butyl}-N-(4-fluorobenzyl)- N-methylamine, 1-{3,4-bis[(9Z,12Z)-octadeca-9,12-dienyloxy]butyl}-4-(2-fluorophenyl)piperazine, N-benzyl-N-{3,4-bis[(9Z,12Z)-octadeca-9,12-dienyloxy]butyl}-N-ethylamine, N-{3,4-bis[(9Z,12Z)-octadeca-9,12-dienyloxy]butyl}-N-ethyl-N′,N′-dimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, N,N-bis(2-methoxyethyl)-3,4-bis[(9Z,12Z)-octadeca-9,12-dienyloxy]butan-1-amine, 1-{3,4-bis[(9Z,12Z)-octadeca-9,12-dienyloxy]butyl}-4-methoxypiperidine, 1-{(3R)-3,4-bis[(9Z,12Z)-octadeca-9,12-dienyloxy]butyl}pyrrolidine, 1-{(3S)-3,4-bis[(9Z,12Z)-octadeca-9,12-dienyloxy]butyl}pyrrolidine, N-{(3R)-3,4-bis[(9Z,12Z)-octadeca-9,12-dienyloxy]butyl}-N,N-diethylamine, N-{(3 S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 2-(2-{3,4-bis[(9Z,12Z)-octadeca-9,12-dienyloxy]butoxy}ethyl)-1-methylpyrrolidine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis[(9Z,12Z)-octadeca-9,12-dienyloxy]butoxy}ethyl)-N,N-dimethylamine, 4-(diethylamino)-2-[(9Z,12Z)-octadeca-9,12-dienoyloxy]butyl(9Z,12Z)-octadeca-9,12-dienoate, 1-(2-{3,4-bis[(9Z,12Z)-octadeca-9,12-dienyloxy]butoxy}ethyl)pyrrolidine, N, N-diethyl-N-(2-{2-[(8Z,1 Z)-heptadeca-8,11-dienyl]-2-[(9Z,12Z)-octadeca-9,12-dienyl]-1,3-dioxolan-4-yl}ethyl)amine, 1-{[(9Z)-octadec-9-enoyloxy]methyl}-3-pyrrolidin-1-ylpropyl(9Z)-octadec-9-enoate, 1-{3,4-bis[(9Z)-octadec-9-enyloxy]butyl}pyrrolidine, 1-{[(5Z,8Z,11Z,14Z)-icosa-5,8,11,14-tetraenoyloxy]methyl}-3-pyrrolidin-1-ylpropyl(5Z,8Z,11Z,14Z)-icosa-5,8,11,14-tetraenoate, (3S)-3,4-bis[(9Z,12Z)-octadeca-9,12-dienyloxy]butyl 3-pyrrolidin-1-ylpropylcarbamate, 1-[3,4-bis(octadecyloxy)butyl]pyrrolidine, 1-[3,4-bis(hexadecyloxy)butyl]pyrrolidine, 1-{3,4-bis[(9E)-hexadec-9-enyloxy]butyl}pyrrolidine, 1-{3,4-bis[(9E)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N¹-{(3S)-3,4-bis[(9Z,12Z)-octadeca-9,12-dienyloxy]butyl}-N³,N³-diethyl-beta-alaninamide, N-{3,4-bis[(9Z,12Z)-octadeca-9,12-dienyloxy]butyl}-N-[3-(1H-imidazol-1-yl)propyl]amine, N-{3,4-bis[(9Z,12Z)-octadeca-9,12-dienyloxy]butyl}-N,N′,N′-trimethylpropane-1,3-diamine, 1-(1-{3,4-bis [(9Z,12Z)-octadeca-9,12-dienyloxy]butyl}pyrrolidin-3-yl)-1H-imidazole, N-{3,4-bis[(9Z,12Z)-octadeca-9,12-dienyloxy]butyl}-N-(3-pyrrolidin-1-ylpropyl)amine, N-{3,4-bis[(9Z,12Z)-octadeca-9,12-dienyloxy]butyl}-N′,N′-dimethylpropane-1,3-diamine, 1-{3,4-bis[(9Z,12Z)-octadeca-9,12-dienyloxy]butyl}azetidine, 1-{3,4-bis[(9Z,12Z)-octadeca-9,12-dienyloxy]butyl}-2-methylpyrrolidine, and 1-{3,4-bis[(9Z,12Z)-octadeca-9,12-dienyloxy]butyl}-2,5-dimethylpyrrolidine.

5. The CaBLES of claim 2, or the Lipid-Based Particle of claim 3, wherein one or more PEG-lipid conjugates are chosen from 2-(tetradecyloxy)-1-((tetradecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate, 2-(hexadecyloxy)-1-((hexadecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate, 2-(octadecyloxy)-1-((octadecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate, 2-2,5,8,11,14,17,20,23,26,29,32,35,38,41,44,47,50,53,56,59,62,65,68,71,74,77,80,83,86,89,92, 94,97,100,103,106,109,112,115,118,121,124,127,130,133,136-hexatetracontaoxaoctatriacontahectanamidopropane-1,3-diyl ditetradecanoate, 2-2,5,8,11,14,17,20,23,26,29,32,35,38,41,44,47,50,53,56,59,62,65,68,71,74,77,80,83,86,89,92, 94,97,100,103,106,109,112,115,118,121,124,127,130,133,136-hexatetracontaoxaoctatriacontahectanamidopropane-1,3-diyl dipalmitate, 2-2,5,8,11,14,17,20,23,26,29,32,35,38,41,44,47,50,53,56,59,62,65,68,71,74,77,80,83,86,89,92, 94,97,100,103,106,109,112,115,118,121,124,127,130,133,136-hexatetracontaoxaoctatriacontahectanamidopropane-1,3-diyl distearate, N-(2-(hexadecyloxy)-1-((hexadecyloxy)methyl)ethyl)-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-(2-(tetradecyloxy)-1-((tetradecyloxy)methyl)ethyl)-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-(2-(octadecyloxy)-1-((octadecyloxy)methyl)ethyl)-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, 6-oxo-2-(tetradecanoyloxy)-8,11,14,17,20,23,26,29,32,35,38,41,44,47,50,53,56,59,62,65,68,71,74,77,80,83,86,89,92,95,9 8,101,104,107,110,113,116,119,122,125,128,131,134,137,140,143-hexatetracontaoxa-5-azatetratetracontahect-1-yl myristate, N-[3,4-bis(tetradecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N- [3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N- [3,4-bis(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, 3,7,11,15,19,23,27,31,35,39,43,47,51,55,59,63,67,71,75,79,83,87,91,95,99,103,107,111,115, 119,123,127,131,135,139,143,147,151,155,159,163,167,171,175,179,182-hexatetracontaoxatrioctacontahect-1-yl 3,4-bis(tetradecyloxy)butylcarbamate, 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-yl 3,4-bis(hexadecyloxy)butylcarbamate, 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-yl 3,4-bis(octadecyloxy)butylcarbamate, N-[3,4-bis(hexadecyloxy)butyl]-N′-3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylsuccinamide, 6-oxo-2-(tetradecanoyloxy)-7,10,13,16,19,22,25,28,31,34,37,40,43,46,49,52,55,58,61,64,67,70,73,76,79,82,85,88,91,94,9 7,100,103,106,109,112,115,118,121,124,127,130,133,136,139,142,145-heptatetracontaoxa-5-azahexatetracontahect-1-yl myristate, 6-oxo-2-(palmitoyloxy)-7,10,13,16,19,22,25,28,31,34,37,40,43,46,49,52,55,58,61,64,67,70,73,76,79,82,85,88,91,94,9 7,100,103,106,109,112,115,118,121,124,127,130,133,136,139,142,145-heptatetracontaoxa-5-azahexatetracontahect-1-yl palmitate, 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate, 6-oxo-2-(palmitoyloxy)-8,11,14,17,20,23,26,29,32,35,38,41,44,47,50,53,56,59,62,65,68,71,74,77,80,83,86,89,92,95,9 8,101,104,107,110,113,116,119,122,125,128,131,134,137,140,143-hexatetracontaoxa-5-azatetratetracontahect-1-yl palmitate, N-[4-(decyloxy)-3-(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-[3,4-bis(decyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, N-[3,4-bis(hexadecyloxy)butyl]-2,5,8,11,14,17,20,23,26,29,32,35,38,41,44,47,50,53,56,59,62,65,68-tricosaoxaheptacontan-70-amide, N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143,146,149,152,155,158, 161,164,167,170,173,176,179,182,185,188,191,194,197,200,203,206,209,212,215,218,221,2 24,227,230,233,236,239,242,245,248,251,254,257,260,263,266,269,272,275,278,281,284,28 7,290,293,296,299,302,305,308,311,314,317,320,323,326,329,332,335,338-113oxa340n-340-amide, N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, 1,2-distearoyl-sn-glycerol-methoxypolyethyleneglycol-750, 1,2-dimyristoyl-sn-glycerol-methoxypolyethyleneglycol-750, 1,2-dipalmitoyl- sn-glycerol-methoxypolyethyleneglycol-750, poly(oxy-1,2-ethanediyl)-2000-α-(3β)-cholest-5-en-3-yl-omega-hydroxy, 1,2-dipalmitoyl-sn-glycerol-methoxypolyethyleneglycol-5000, poly(oxy-1,2-ethanediyl)-5000-α-(3β)-cholest-5-en-3-yl-omega-hydroxy, (2S,3R,E)-3-hydroxy-2-stearamidooctadec-4-enyl polyethyleneglycol-2000 methyl ether succinate, (2S,3R,E)-3-hydroxy-2-icosanamidooctadec-4-enyl polyethyleneglycol-2000 methyl ether succinate, N-(2,3-dimyristyloxypropyl)carbamate polyethyleneglycol-2000 methyl ether, N-(carbonylmethoxypolyethyleneglycol-750)-1,2-dimyristoyl-sn-glycero-phosphatidylethanolamine, N-(carbonyl-methoxypolyethyleneglycol-750)-i,2-distearoyl-sn-glycero-3-phosphoethanolamine, N-(carbonyl-methoxypolyethyleneglycol-750)-1,2-dipalmitoyl-sn-glycero-3-phosphoethanolamine, N-(carbonyl-methoxypolyethyleneglycol-2000)-1,2-dimyristoyl-sn-glycero-3-phosphoethanolamine, N-(carbonyl-methoxypolyethyleneglycol-2000)-1,2-dipalmitoyl-sn-glycero-3-phosphoethanolamine, N-(carbonyl-methoxypolyethyleneglycol-2000)-1,2-distearoyl-sn-glycero-3-phosphoethanolamine, N-(carbonyl-methoxypolyethyleneglycol-2000)-dioleoyl-phosphatidylethanolamine, 1,2-distearoyl-sn-glycerol-methoxypolyethyleneglycol-2000, 1,2-dimyristoyl-sn-glycerol-methoxypolyethyleneglycol-2000, 1,2-dipalmitoyl-sn-glycerol-methoxypolyethyleneglycol-2000, mPEG-2000-cholesterol, octanoyl-mPEG-2000-ceramide, palmitoyl-mPEG-2000-ceramide, N-(carbonyl-methoxypolyethyleneglycol-5000)-1,2-dimyristoyl-sn-glycero-3-phosphoethanolamine, N-(carbonyl-methoxypolyethyleneglycol-5000)-1,2-dipalmitoyl-sn-glycero-3-phosphoethanolamine, N-(carbonyl-methoxypolyethyleneglycol-5000)-1,2-distearoyl-sn-glycero-3-phosphoethanolamine, 1,2-dimyristoyl-sn-glycerol-methoxypolyethyleneglycol-5000, 1,2-distearoyl-sn-glycerol-methoxypolyethyleneglycol-5000, mPEG-5000-cholesterol, octanoyl-mPEG-5000-ceramide, or palmitoyl-mPEG-5000-ceramide.

6. The Lipid-Based Particle of claim 3, wherein the therapeutic agent is RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), an antigen, fragments thereof, a protein, a peptide, small-molecules, or mixtures thereof.

7. A pharmaceutical composition comprising a Lipid-Based Particle of claim 3 and a pharmaceutically acceptable carrier.

8. The Lipid-Based Particle of claim 3, wherein said non-cationic lipids are cholesterol and D SPC, said cationic lipid is 1-{(3S)-3,4-bis[(9Z,12Z)-octadeca-9,12-dienyloxy]butyl}pyrrolidine, said PEG-lipid conjugate is N-(2,3-dimyristyloxypropyl)carbamate polyethyleneglycol-2000 methyl ether, and said therapeutic agent is siRNA.

9. The Lipid-Based Particle of claim 3, wherein said non-cationic lipids are cholesterol and DSPC, said cationic lipid is 1-{(3R)-3,4-bis[(9Z,12Z)-octadeca-9,12-dienyloxy]butyl}pyrrolidine, said PEG-lipid conjugate is N-(2,3-dimyristyloxypropyl)carbamate polyethyleneglycol-2000 methyl ether, and said therapeutic agent is siRNA.

10. The Lipid-Based Particle of claim 3, wherein said non-cationic lipids are cholesterol and DSPC, said cationic lipid is 1-{3,4-bis[(9Z,12Z)-octadeca-9,12-dienyloxy]butyl}-N,N-dimethylpyrrolidin-3-amine, said PEG-lipid conjugate is N-(2,3-dimyristyloxypropyl)carbamate polyethyleneglycol-2000 methyl ether, and said therapeutic agent is siRNA.

11. The Lipid-Based Particle of claim 3, wherein said non-cationic lipids are cholesterol and DSPC, said cationic lipid is 1-{3,4-bis[(9Z,12Z)-octadeca-9,12-dienyloxy]butyl}pyrrolidine, said PEG-lipid conjugate is 2-(octadecyloxy)-1-((octadecyloxy)methyl)ethyl 3,6,9,12,15,18,21,24,27,30,33,36,39,42,45,48,51,54,57,60,63,66,69,72,75,78,81,84,87,90,93, 96,99,102,105,108,111,114,117,120,123,126,129,132,135,138-hexatetracontaoxanonatriacontahect-1-ylcarbamate, and said therapeutic agent is siRNA.

12. The Lipid-Based Particle of claim 3, wherein said non-cationic lipids are cholesterol and DSPC, said cationic lipid is 1-{3,4-bis[(9Z,12Z)-octadeca-9,12-dienyloxy]butyl}pyrrolidine, said PEG-lipid conjugate is N-[3,4-bis(hexadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, and said therapeutic agent is siRNA.

13. The Lipid-Based Particle of claim 3, wherein said non-cationic lipids are cholesterol and DSPC, said cationic lipid is 1-{3,4-bis[(9Z,12Z)-octadeca-9,12-dienyloxy]butyl}pyrrolidine, said PEG-lipid conjugate is N-[3,4-bis(octadecyloxy)butyl]-2,5,8,11,14,17,20,23,26,29,32,35,38,41,44,47,50,53,56,59,62,65,68,71,74,77,80,83,86,89,92, 95,98,101,104,107,110,113,116,119,122,125,128,131,134,137-hexatetracontaoxanonatriacontahectan-139-amide, and said therapeutic agent is siRNA.

14. The Lipid-Based Particle of claim 3, wherein said non-cationic lipids are cholesterol and DSPC, said cationic lipid is 1-{3,4-bis[(9Z,12Z)-octadeca-9,12-dienyloxy]butyl}pyrrolidine, said PEG-lipid conjugate is 1,2-dipalmitoyl-sn-glycerol-methoxypolyethyleneglycol-2000, and said therapeutic agent is siRNA.

15. The Lipid-Based Particle of claim 3, wherein said non-cationic lipids are cholesterol and DSPC, said cationic lipid is 1-{3,4-bis[(9Z,12Z)-octadeca-9,12-dienyloxy]butyl pyrrolidine, said PEG-lipid conjugate is N-(carbonyl-methoxypolyethyleneglycol-2000)-1,2-dipalmitoyl-sn-glycero-3-phosphoethanolamine, and said therapeutic agent is siRNA.

16. A method of making the Lipid-Based Particle of claim 3, comprising:
(a) mixing the cationic lipid(s), the non-cationic lipid(s) and the PEG-lipid conjugate(s);
(b) adding the mixture of step (a) to one or more therapeutic agents; and
(c) separating and purifying resulting suspension of step (b).

17. The method of claim 37, wherein said mixture of step (a) and one or more said therapeutic agents are warmed to about 60° C. prior to the addition of said mixture of step (a) to one or more therapeutic agents via needle injection.

18. The CaBLES of claim 2 used to deliver a therapeutic agent wherein one or more cationic lipids are chosen from 1-{3,4-bis[(9Z,12Z)-octadeca-9,12-dienyloxy]butyl}piperidine, 4-{3,4-bis[(9Z,12Z)-octadeca-9,12-dienyloxy]butyl}morpholine, N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, 1-{3,4-bis[(9Z,12Z)-octadeca-9,12-dienyloxy]butyl}-4-phenylpiperazine, 1-{3,4-bis[(9Z,12Z)-octadeca-9,12-dienyloxy]butyl}-4-methylpiperazine, N-(2-methoxyethyl)-N-methyl-3,4-bis [(9Z,12Z)-octadeca-9,12-dienyloxy]butan-1-amine, 1-{3,4-bis[(9Z,12Z)-octadeca-9,12-dienyloxy]butyl}-4-(2-methoxyphenyl)piperazine, N-{3,4-bis[(9Z,12Z)-octadeca-9,12-dienyloxy]butyl}-N,N′,N′-trimethylethane-1,2-diamine, N-{3,4-bis[(9Z,12Z)-octadeca-9,12-dienyloxy]butyl}-N-methyl-N-(2-pyridin-2-ylethyl)amine, N-benzyl-N-{3,4-bis[(9Z,12Z)-octadeca-9,12-dienyloxy]butyl}-N-methylamine, N-{3,4-bis[(9Z,12Z)-octadeca-9,12-dienyloxy]butyl}-N-(4-fluorobenzyl)-N-methylamine, 1-{3,4-bis[(9Z,12Z)-octadeca-9,12-dienyloxy]butyl}-4-(2-fluorophenyl)piperazine, N-benzyl-N-{3,4-bis[(9Z,12Z)-octadeca-9,12-dienyloxy]butyl}-N-ethylamine, N-{3,4-bis[(9Z,12Z)-octadeca-9,12-dienyloxy]butyl}-N-ethyl-N′,N′-dimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, N,N-bis(2-methoxyethyl)-3,4-bis[(9Z,12Z)-octadeca-9,12-dienyloxy]butan-1-amine, 1-{3,4-bis[(9Z,12Z)-octadeca-9,12-dienyloxy]butyl}-4-methoxypiperidine, 1-{(3R)-3,4-bis[(9Z,12Z)-octadeca-9,12-dienyloxy]butyl}pyrrolidine, 1-{(3S)-3,4-bis[(9Z,12Z)-octadeca-9,12-dienyloxy]butyl}pyrrolidine, N-{(3R)-3,4-bis[(9Z,12Z)-octadeca-9,12-dienyloxy]butyl}-N,N-diethylamine, N-{(3S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 2-(2-{3,4-bis[(9Z,12Z)-octadeca-9,12-dienyloxy]butoxy}ethyl)-1-methylpyrrolidine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis [(9Z,12Z)-octadeca-9,12-dienyloxy]butoxy}ethyl)-N,N-dimethylamine, 4-(diethylamino)-2-[(9Z,12Z)-octadeca-9,12-dienoyloxy]butyl (9Z,12Z)-octadeca-9,12-dienoate, 1-(2-{3,4-bis[(9Z,12Z)-octadeca-9,12-dienyloxy]butoxy}ethyl)pyrrolidine, N,N-diethyl-N-(2-{2-[(8Z,11Z)-heptadeca-8,11-dienyl]-2-[(9Z,12Z)-octadeca-9,12-dienyl]-1,3-dioxolan-4-yl}ethyl)amine, 1-{[(9Z)-octadec-9-enoyloxy]methyl}-3-pyrrolidin-1-ylpropyl (9Z)-octadec-9-enoate, 1-{3,4-bis[(9Z)-octadec-9-enyloxy]butyl}pyrrolidine, 1-{[(5Z,8Z,11Z,14Z)-icosa-5,8,11,14-tetraenoyloxy]methyl}-3-pyrrolidin-1-ylpropyl (5Z,8Z,11Z,14Z)-icosa-5,8,11,14-tetraenoate, (3S)-3,4-bis[(9Z,12Z)-octadeca-9,12-dienyloxy]butyl 3-pyrrolidin-1-ylpropylcarbamate, 1-[3,4-bis(octadecyloxy)butyl]pyrrolidine, 1-[3,4-bis(hexadecyloxy)butyl]pyrrolidine, 1-{3,4-bis[(9E)-hexadec-9-enyloxy]butyl}pyrrolidine, 1-{3,4-bis[(9E)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N¹-{(3S)-3,4-bis[(9Z,12Z)-octadeca-9,12-dienyloxy]butyl}-N³,N³-diethyl-beta-alaninamide, N-{3,4-bis[(9Z,12Z)-octadeca-9,12-dienyloxy]butyl}-N-[3-(1H-imidazol-1-yl)propyl]amine, N-{3,4-bis[(9Z,12Z)-octadeca-9,12-dienyloxy]butyl}-N,N′,N′-trimethylpropane-1,3-diamine, 1-(1-{3,4-bis [(9Z,12Z)-octadeca-9,12-dienyloxy]butyl}pyrrolidin-3-yl)-1H-imidazole, N-{3,4-bis[(9Z,12Z)-octadeca-9,12-dienyloxy]butyl}-N-(3-pyrrolidin-1-ylpropyl)amine, N-{3,4-bis[(9Z,12Z)-octadeca-9,12-dienyloxy]butyl}-N′,N′-dimethylpropane-1,3-diamine, 1-{3,4-bis[(9Z,12Z)-octadeca-9,12-dienyloxy]butyl}azetidine, 1-{3,4-bis[(9Z,12Z)-octadeca-9,12-dienyloxy]butyl}-2-methylpyrrolidine, and 1-{3,4-bis[(9Z,12Z)-octadeca-9,12-dienyloxy]butyl}-2,5-dimethylpyrrolidine.

19. The CaBLES of claim 2 used to deliver a therapeutic agent wherein one or more cationic lipids are chosen from 1-{(3S)-3,4-bis[(9Z,12Z)-octadeca-9,12-dienyloxy]butyl}pyrrolidine, 1-{(3R)-3,4-bis[(9Z,12Z)-octadeca-9,12-dienyloxy]butyl}pyrrolidine, 1-{3,4-bis[(9Z,12Z)-octadeca-9,12-dienyloxy]butyl}-N,N-dimethylpyrrolidin-3-amine, and 1-{3,4-bis[(9Z,12Z)-octadeca-9,12-dienyloxy]butyl}pyrrolidine.

20. The CaBLES of claim 2 used to deliver a therapeutic agent wherein one or more cationic lipids are chosen from N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, N-(2-methoxyethyl)-N-methyl-3,4-bis[(9Z,12Z)-octadeca-9,12-dienyloxy]butan-1-amine, N-{3,4-bis[(9Z,12Z)-octadeca-9,12-dienyloxy]butyl}-N,N′,N′-trimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, 1-{(3R)-3,4-bis[(9Z,12Z)-octadeca-9,12-dienyloxy]butyl}pyrrolidine, 1-{(3S)-3,4-bis[(9Z,12Z)-octadeca-9,12-dienyloxy]butyl}pyrrolidine, N-{(3R)-3,4-bis[(9Z,12Z)-octadeca-9,12-dienyloxy]butyl}-N,N-diethylamine, N-{(3S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis [(9Z, 12Z)-octadeca-9,12-dienyloxy]butoxy}ethyl)-N,N-dimethylamine, 1-(2-{3,4-bis[(9Z,12Z)-octadeca-9,12-dienyloxy]butoxy}ethyl)pyrrolidine, N,N-diethyl-N-(2-{2-[(8Z,11Z)-heptadeca-8,11-dienyl]-2-[(9Z,12Z)-octadeca-9,12-dienyl]-1,3-dioxolan-4-yl}ethyl)amine, 1-{3,4-bis[(9Z)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N-{3,4-bis[(9Z,12Z)-octadeca-9,12-dienyloxy]butyl}-N,N′,N′-trimethylpropane-1,3-diamine, and 1-{3,4-bis[(9Z,12Z)-octadeca-9,12-dienyloxy]butyl}azetidine.

21. The CaBLES of claim 2 or the Lipid-Based Particle of claim 3 wherein a ligand is attached to the terminus of one or more (PEG)-lipid conjugates.
